SEQUENCE LISTING (5 sequences)

SEQ ID NO: 1 - RNA, 1184 nt, synthetic construct
  source 1..1184: mol_type = other RNA
actcttctggtccccacagactcagagagaacccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaaaaaaaaaaaaaaaaaaaaaaaatgcataaaaaaaaaaaaaaaaaaaaaaaaaaaaaaaaaaaaaaaaaaaaaaaaaaaaaaaaaaaa

SEQ ID NO: 2 - RNA, 415 nt, synthetic construct
  source 1..415: mol_type = other RNA
gggtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggccctatagtgagtcgtatta

SEQ ID NO: 3 - RNA, 21 nt, synthetic construct
  source 1..21: mol_type = other RNA
ttccgtatgttgcatcacctt

SEQ ID NO: 4 - RNA, 21 nt, synthetic construct
  source 1..21: mol_type = other RNA
ggtgatgcaacatacggaatt

SEQ ID NO: 5 - protein, 12 aa, unidentified
PKKKRKVEDPYC

(19) United States
(12) Patent Application Publication
(10) Pub. No.: US 2026/0357276 A1
(43) Pub. Date: Nov. 12, 2026

(54) PROTEIN-COATED LDH NANOPARTICLES AS NANOCARRIERS IN PLANTS

(71) Applicant: THE UNIVERSITY OF QUEENSLAND, Queensland (AU)

(72) Inventors: Wang XU, Queensland (AU); Zhi Ping XU, Queensland (AU); Neena MITTER, Queensland (AU); Bernard CARROLL, Queensland (AU); Jiaxi YONG, Queensland (AU)

(21) Appl. No.: 19/487,599

(22) PCT Filed: May 27, 2024
(86) PCT No.: PCT/AU2024/050554

§ 371 Date: Nov. 25, 2025

(30) Foreign Application Priority Data
May 26, 2023 (AU) 2023901657

(51) Int. Cl. C12N 15/82; C07K 14/005; C07K 17/14; C12N 9/36; C12N 15/88

(52) U.S. Cl. CPC C12N 15/8206; C07K 14/005; C07K 17/14; C12N 9/2462; C12N 15/88; C12N 2710/22022; C12N 2810/6009; C12Y 302/01017

(57) ABSTRACT

The present disclosure relates generally to a composition comprising nucleic acids adsorbed onto layered double hydroxide (LDH) nanoparticles coated in positively-charged protein, and methods of using same to introduce the adsorbed nucleic acids to plants and plant tissues. For example, the LDH nanoparticles of the disclosure may be used to deliver nucleic acids of interest to plants or parts thereof for a range of purposes, including, but not limited to, protecting a plant against pests and diseases, plant trait manipulation, and/or plant gene editing. The present disclosure also relates generally to the use of lysozyme to enhance uptake and internalisation of LDH nanoparticles by plants and plant tissues. For example, the disclosure relates to methods of enhancing uptake and internalisation of LDH nanoparticles by plants and plant tissues by pre-treating the plant or plant tissue with lysozyme prior to incubating the plant or plant tissue with the LDH nanoparticles. The present disclosure also relates to methods for preparing said compositions.

### RELATED APPLICATION DATA

The present application claims priority from Australian Provisional Patent Application No 2023901657 filed on 26 May 2023, the content of which is incorporated herein by reference in its entirety.

### TECHNICAL FIELD

The present disclosure relates generally to a composition comprising nucleic acids adsorbed onto layered double hydroxide (LDH) nanoparticles coated in positively-charged protein, and methods of using same to introduce the adsorbed nucleic acids to plants and plant tissues. For example, the LDH nanoparticles of the disclosure may be used to deliver nucleic acids of interest to plants or parts thereof for a range of purposes, including, but not limited to, protecting a plant against pests and diseases, plant trait manipulation, and/or plant gene editing. The present disclosure also relates generally to the use of lysozyme to enhance uptake and internalisation of LDH nanoparticles by plants and plant tissues. For example, the disclosure relates to methods of enhancing uptake and internalisation of LDH nanoparticles by plants and plant tissues by pre-treating the plant or plant tissue with lysozyme prior to incubating the plant or plant tissue with the LDH nanoparticles. The present disclosure also relates to methods for preparing said compositions.

### BACKGROUND

To address global food security and feed a growing world population, food production needs to be increased by at least 50% by 2050. In this context, pests and pathogens are estimated to reduce agricultural productivity by >40%, while food security is threatened further by increasing abiotic stress due to climate change. Currently, crop improvement and crop protection rely on traditional plant breeding, genetic modification (GM), and the use of toxic fungicides and insecticides. GM methods involving the use of transgenes encoding specific proteins, small interfering RNA (siRNA), artificial microRNA, CRISPR Cas9 and guide RNA (gRNA) have been successfully introduced to manipulate important physiological traits, however the process is time-consuming and extremely difficult to achieve for many important crop species. A further challenge in plant biotechnology is that exogenous biomolecules must penetrate through the rigid plant cell wall in order to enter the cell and confer their biological functions. The engineering of crop species currently requires biolistic bombardment-based delivery of plasmid DNA or agrobacteria-mediated gene transfer into cultured plant cells, followed by plant regeneration. However, biolistic bombardment has a low transfection efficiency and causes severe damage to the plant cells and fragmentation of the genome, while agrobacteria- and virus-mediated gene delivery are limited to certain host plant species. Additionally, plant tissue culture itself causes collateral genetic and/or epigenetic damage to the plant genome. Further, the acceptance of GM crops and chemical pesticides is faltering, and a transition to non-GM and chemical pesticide-free agriculture is gaining considerable traction globally, especially in Europe.

### SUMMARY

The present disclosure is based, inter alia, on the recognition by the inventors that there is a need to provide effective and alternative approaches for crop improvement, enhancing the quality and value of crop and horticultural produce, and the control of plant viruses, parasites, insects, nematodes or fungal infections, including approaches that are non-GM and minimally- or non-toxic. Nanoparticle-mediated delivery of functional macro-biomolecules, such as RNA and DNA, into intact plants represents a new non-GM, environmentally friendly strategy with the immediate potential to facilitate a transition away from the use of harmful chemicals in agriculture. Furthermore, nanoparticle-mediated delivery of functional macro-biomolecules represents a non-infectious and non-transgenic approach for expressing a desired protein, peptide or non-coding RNA in an organism of interest e.g., plants, and has the potential to manipulate metabolic properties and confer desirable traits in crop plants, such as traits relating to crop yield, food and harvest quality, and pest resistance.

Layered double hydroxide (LDH) nanoparticles are a family of clay materials that have been widely investigated for biomolecule delivery in biomedical applications. However, the plant cell wall provides a more challenging barrier to the delivery of large biomolecules into plant cells and nanoparticles have so far not been well-designed to achieve active uptake in plants. In previous studies on nanoparticle-based delivery to plants, direct foliar application, application to developing pollen or cultured plant cells, or injection directly into plant tissues have been used. However, nanoparticle delivery via hydroponic culture media into intact plant roots and tissues responsible for taking up exogenous nutrients in solution from the soil have been less studied. Here, the inventors demonstrated that coating of LDH nanoparticles with a protein, polypeptide or peptide having an overall positive surface charge stabilizes the LDH nanoparticles and reduces their aggregation on the surface of the plant tissue and in culture media, resulting in improved uptake and cell-to-cell translocation of the nanoparticles. Without being bound by any one theory, the inventors believe that the improved uptake, internalisation and cell-to-cell translocation observed for the coated nanoparticles is, in part, due to the ability of the peptide- or protein-coated LDH nanoparticles (hereinafter referred to collectively as “protein-coated LDH nanoparticle”, “protein-coated LDH nanoparticles” or similar) to access the endocytosis and membrane trafficking pathways of plants.

Using lysozyme and a SV40 T-antigen nuclear localisation signal (NLS) peptide as exemplary proteins for coating of LDH nanoparticles, the inventors have been able to efficiently deliver various forms of functional nucleic acids into plant cells, plant tissues and intact plants, for example, i) mRNA into N. benthamiana, Arabidopsis thaliana, tomato, and sorghum roots, leaf, pollen and/or callus, where it is subsequently translated into protein; ii) plasmid DNA into N. benthamiana, A. thaliana and sorghum roots, where it is efficiently expressed into protein; iii) PCR amplified DNA fragments into A. thaliana roots, where it is efficiently expressed into protein; and iv) small interfering RNA and long double-stranded RNA into intact N. benthamiana roots to induce RNA interference of a target gene. These lysozyme- and/or NPS-coated LDH nanoparticles were not only internalized by plant root cells, but also translocated to the vascular cylinder and above-ground tissues.

The inventors have also used the protein-coated LDH nanoparticle delivery system to deliver plasmid DNA encoding a functional mRNA transcript for GFP containing a mobile RNA motif in the 3′ UTR to intact roots of A. thaliana, and showed systemic expression of GFP in root tip, mature root, stem and leaf, even in the presence of graft junctions. In addition to the use of protein coating to enhance uptake and internalisation of LDH nanoparticles to plant tissue, the inventors have also used pre-treatment of intact roots of A. thaliana and N. benthamiana, and early bicellular pollen of tomato, with lysozyme prior to incubation with LDH nanoparticles and shown that the lysozyme pre-treatment enhances uptake and internalisation of the protein-coated and uncoated LDH nanoparticles to plant tissue.

Thus, the inventors have successfully demonstrated that LDH nanoparticles coated with a protein having an overall positive charge are able to deliver large functional biomolecules into intact roots, leaf tissue and pollen of monocot and dicot plants, and facilitate the subsequent systemic movement of exogenous biomolecules which have the potential to manipulate biological processes in the above-ground shoots of the plant. The enhanced uptake and internalisation of LDH nanoparticles coated with protein having an overall positive charge is due, in part, to the positive surface charge enhancing the affinity of the nanoparticles for the negatively charged cell surface and/or cytoplasm immediately inside the cell membrane and stimulating the membrane trafficking pathway. Furthermore, partial degradation of polysaccharides in the plant cell wall by lysozyme, whether coated on LDH nanoparticles or used as a pre-treatment for tissue, allows the nanoparticles to diffuse more freely through the apoplast and readily engage in endocytosis via the cell membrane.

Accordingly, in one example, the present disclosure provides a composition comprising one or more layered double hydroxide (LDH) nanoparticles onto which one or more nucleic acid molecules are adsorbed and which are coated with protein having an overall positive surface charge.

The present disclosure also provides a method for preparing the composition described herein, the method comprising adsorbing one or more nucleic acids molecules onto an LDH nanoparticle, and then coating the LDH nanoparticles with a protein that has an overall positive surface charge.

In one example, the one or more LDH nanoparticles are encapsulated by the protein that has an positive overall surface charge. In one example, the one or more nucleic acid molecule adsorbed to the one or more LDH nanoparticles are encapsulated by the protein that has an overall positive surface charge.

The present disclosure also provides a method of introducing one or more nucleic acid molecules to a plant tissue, comprising contacting the plant tissue with a protein having an overall positive surface charge and one or more layered double hydroxide (LDH) nanoparticles onto which the one or more nucleic acid molecules are adsorbed.

In one example, the method of introducing one or more nucleic acid molecules to a plant tissue comprises contacting the plant tissue with the protein and LDH nanoparticle separately. For example, the protein and LDH nanoparticle may be contacted with the plant tissue consecutively. In some examples, the plant tissue is contacted with the one or more LDH nanoparticles prior to being contacted with the protein. In some examples, the plant tissue is contacted with the protein prior to being contacted with the one or more LDH nanoparticles. In accordance with an example in which the plant tissue is contacted with the protein prior to being contacted with the one or more LDH nanoparticles i.e., protein pre-treatment, the protein pre-treatment may commence 15 mins or more prior to contacting the plant tissue with the one or more LDH nanoparticles. For example, the protein pre-treatment may commence about 30 min to about 12 hours prior to contacting the plant tissue with the one or more LDH nanoparticles. For example, the protein pre-treatment may commence about 1 hour prior to contacting the plant tissue with the one or more LDH nanoparticles. For example, the protein pre-treatment may commence about 2 hours prior to contacting the plant tissue with the one or more LDH nanoparticles. For example, the protein pre-treatment may commence about 3 hours prior to contacting the plant tissue with the one or more LDH nanoparticles. For example, the protein pre-treatment may commence about 4 hours prior to contacting the plant tissue with the one or more LDH nanoparticles. For example, the protein pre-treatment may commence about 5 hours prior to contacting the plant tissue with the one or more LDH nanoparticles. For example, the protein pre-treatment may commence about 6 hours prior to contacting the plant tissue with the one or more LDH nanoparticles. For example, the protein pre-treatment may commence about 7 hours prior to contacting the plant tissue with the one or more LDH nanoparticles. For example, the protein pre-treatment may commence about 8 hours prior to contacting the plant tissue with the one or more LDH nanoparticles. For example, the protein pre-treatment may commence about 9 hours prior to contacting the plant tissue with the one or more LDH nanoparticles. For example, the protein pre-treatment may commence about 10 hours prior to contacting the plant tissue with the one or more LDH nanoparticles. For example, the protein pre-treatment may commence about 11 hours prior to contacting the plant tissue with the one or more LDH nanoparticles. For example, the protein pre-treatment may commence about 12 hours prior to contacting the plant tissue with the one or more LDH nanoparticles. In each of the foregoing examples describing protein pre-treatment, the plant tissue may be in contact with the protein (e.g., co-incubated) for 15 min or more. For example, the protein pre-treatment may comprise contacting the plant tissue with the protein for between about 30 min to about 6 hours. For example, the protein pre-treatment may comprise contacting the plant tissue with the protein for about 1 hour. For example, the protein pre-treatment may comprise contacting the plant tissue with the protein for about 2 hours. For example, the protein pre-treatment may comprise contacting the plant tissue with the protein for about 3 hours. For example, the protein pre-treatment may comprise contacting the plant tissue with the protein for about 4 hours. For example, the protein pre-treatment may comprise contacting the plant tissue with the protein for about 5 hours. For example, the protein pre-treatment may comprise contacting the plant tissue with the protein for about 6 hours.

In other examples, the method of introducing one or more nucleic acid molecules to a plant tissue comprises contacting the plant tissue with the protein having an overall positive surface charge and the one or more LDH nanoparticles onto which the one or more nucleic acid molecules are adsorbed simultaneously. For example, the one or more LDH nanoparticles may be coated with the protein and contacted with the plant tissue. Alternatively, the one or more LDH nanoparticles and the protein having an overall positive surface charge may be provided separately and contacted with the plant tissue simultaneously.

In each of the foregoing examples describing compositions and methods of the disclosure, the one or more nucleic acid molecules may be single-stranded nucleic acid molecules. Alternatively, the one or more nucleic acid molecules may be double-stranded nucleic acid molecules. Exemplary nucleic acid molecules may be selected from the group consisting of a messenger RNA (mRNA), a double stranded RNA (dsRNA), a small interfering RNA (siRNA), in single stranded RNA (ssRNA), transfer RNA (tRNA), microRNA (miRNA), precursor microRNA (pre-miRNA), primary microRNA (pri-microRNA), short hairpin microRNA (shmiR), short hairpin RNA (shRNA), ribosomal RNA (rRNA), small guide RNA (sgRNA), a plasmid DNA (pDNA), PCR-amplified DNA and any combination thereof.

In one example, the one or more nucleic acid molecules attached to the surface of the LDH nanoparticle encode a protein or polypeptide of interest or a functional fragment thereof. In accordance with this example, the one or more nucleic acid molecules may be messenger RNA (mRNA), PCR-amplified DNA, or plasmid DNA (pDNA) molecules comprising a polynucleotide sequence encoding the protein or polypeptide of interest or functional fragment thereof. In some examples, the polynucleotide sequence encoding the protein or polypeptide of interest, or functional fragment thereof, further comprises a mobile transfer RNA (tRNA), such as in the 3′ UTR.

In one example, one or more of the nucleic acid molecules attached to the surface of the LDH nanoparticle are RNA interference (RNAi) molecules, each comprising a polynucleotide sequence which is substantially complementary to a mRNA transcript of a target gene in a plant. For example, the RNAi molecule may be selected from a dsRNA, a siRNA, ssRNA, a DNA molecule encoding a pre-miRNA, a DNA molecule encoding a pri-miRNA, a DNA molecule encoding an shmiR a DNA molecule encoding an shRNA or any combination thereof. The target gene may be associated with a plant pathogen or plant pest. The target gene may be associated with an agronomic trait of interest in the plant. The target gene may be associated with an ornamental trait of interest.

In one example, one or more of the nucleic acid molecules attached to the surface of the LDH nanoparticle are capable of modulating expression of a target gene within a plant cell. For example, the nucleic acid molecule capable of modulating expression of a target gene may be selected from a tRNA, a miRNA and rRNA or any combination thereof. The target gene may be associated with a plant pathogen or plant pest. The target gene may be associated with an agronomic trait of interest in the plant. The target gene may be associated with an ornamental trait of interest.

In each of the foregoing examples describing compositions and methods of the disclosure, the protein having an overall positive surface charge is capable of stimulating the endocytosis and/or membrane trafficking pathways in a plant or plant tissue. In one example, the protein having an overall positive surface charge may be capable of degrading polysaccharides in a plant cell wall. In one example, the protein is an enzyme. Exemplary enzymes which are capable of degrading polysaccharides are known in the art and described herein. However, in one particular example, the protein having an overall positive surface charge is lysozyme. In another example, the protein having an overall positive surface charge is, or comprises, a nuclear localization sequence (NLS) peptide. For example, the protein having an overall positive surface charge may be, or comprise, a NLS peptide derived from a Simian virus 40 (SV40) T antigen. For example, the protein having an overall positive surface charge may comprise or consist of the amino acid sequence set forth in SEQ ID NO: 5.

In accordance with certain examples describing the method of the disclosure, the plant tissue is contacted with lysozyme prior to being contacted with the one or more LDH nanoparticles i.e., lysozyme pre-treatment, and the one or more LDH nanoparticles are coated with a further protein having an overall positive surface charge e.g., as described herein, which is not lysozyme. In one example, the further protein having an overall positive surface charge is, or comprises, a NLS peptide derived from a Simian virus 40 (SV40) T antigen. For example, the further protein having an overall positive surface charge may comprise or consist of the amino acid sequence set forth in SEQ ID NO: 5. The lysozyme pre-treatment may be performed according to the protein pre-treatment described herein. Accordingly, exemplary conditions described hereinabove in the context of “protein pre-treatment” shall be taken to apply mutatis mutandis to each and every example describing examples of the method in which the plant tissue is contacted with lysozyme prior to being contacted with the one or more LDH nanoparticles i.e., “lysozyme pre-treatment”.

In each of the foregoing examples describing compositions and methods of the disclosure, the one or more LDH nanoparticles may have an average diameter from about 30 nm to about 50 nm. For example, the one or more LDH nanoparticles may have an average diameter of about 30 nm. For example, the one or more LDH nanoparticles may have an average diameter of about 50 nm.

In each of the foregoing examples describing compositions and methods of the disclosure, the one or more LDH nanoparticles may be coated with the protein at a mass ratio of about 1:5.

According to one example describing the method of introducing one or more nucleic acid molecules to a plant tissue, the protein and the one or more LDH nanoparticles are contacted with the plant tissue in the form of a composition as described herein (i.e., a composition comprising one or more LDH nanoparticles onto which one or more nucleic acid molecules are adsorbed and coated with a protein having an overall positive surface charge).

The method of introducing one or more nucleic acid molecules to a plant tissue may comprise immersing the plant tissue in an incubation medium comprising the LDH nanoparticles in the presence of the protein. In some examples, the protein is coated on the LDH nanoparticles as described herein. In some example, the plant tissue is coated or contacted with the protein prior to immersion of the plant tissue in the medium (such as in a pre-treatment as described herein). In other examples, protein is present in the incubation medium itself.

In accordance with any of the foregoing examples describing a method of introducing one or more nucleic acid molecules to a plant tissue, the plant tissue may be of, or from, a plant that is a monocotyledon (monocot) or a dicotyledon (dicot). In one example, the plant may be selected from the group consisting of a fruiting plant, a leguminous plant, an oil plant, a vegetable plant, a cereal plant, a fibre plant, an ornamental plant, a forestry plant, an aquatic plant, a medicinal plant and a noxious plant or weed. The plant tissue may be of, or from, a root, seed, pollen, ovule, plant cutting, fruit, leaf, flower tissue or scion. In one example, the plant tissue is a root tissue, leaf tissue or pollen of an intact plant. For example, the plant tissue is a root tissue of an intact plant. For example, the plant tissue is pollen of an intact plant. For example, the plant tissue is a leaf tissue of an intact plant. In accordance with examples in which the one or more nucleic acid molecules are introduced to plant tissue of an intact plant, the plant may have previously undergone grafting. Where the plant has previously undergone grafting, the one or more nucleic acid molecules may be introduced to a rootstock tissue of the plant, a scion tissue of the plant, or both. In one example, the one or more nucleic acid molecules are introduced to a rootstock tissue of the plant.

In accordance with examples describing a method of introducing one or more nucleic acid molecules to a plant tissue, the LDH nanoparticles are internalized into the plant tissue and the nucleic acids adsorbed to the LDH nanoparticles are delivered to the plant tissue. In certain examples, the LDH nanoparticles and nucleic acid molecules adsorbed thereto are taken up and internalised by the plant tissue via a membrane trafficking pathway of the plant. In certain examples, the LDH nanoparticles and nucleic acid molecules adsorbed thereto are taken up and internalised by the plant tissue via an endocytosis pathway of the plant. In certain examples, the LDH nanoparticles and nucleic acid molecules adsorbed thereto are taken up and internalised by the plant tissue via endocytosis and a membrane trafficking pathway of the plant. In one example, uptake and/or internalization of the LDH nanoparticles and the one or more nucleic acid molecules adsorbed thereto by the plant tissue following performance of the method is increased or enhanced relative to a method in which the plant tissue is contacted with the LDH nanoparticles in the absence of the protein having an overall positive surface charge. Alternatively, or in addition, the level of aggregation of the LDH nanoparticles is decreased during performance of the method of the disclosure relative to a corresponding method in which the plant tissue is contacted with the LDH nanoparticles in the absence of the protein.

In some examples, the method of the disclosure comprises introducing to the plant one or more nucleic acid molecules which silence expression of a gene in the plant, or modulate expression a gene in the plant, or edit the plant's genome, or increase expression of a gene in the plant or direct expression of an exogenous gene in the plant.

In one example, introduction of the nucleic acid molecule to the plant tissue increases yield, quality and/or harvest value of the plant or a part thereof and/or protects the plant or a part thereof from a plant pest and/or improves resistance of the plant or a part thereof to an abiotic stress and/or a biotic stress. For example, the part of the plant may selected from the group consisting of leaves, stems, cutting and scion, flowers, fruits, nuts, roots, seed, seed coat, embryos and combinations thereof. The plant pest may comprise an insect pest, a viral pathogen, a fungal pathogen, a nematode, a bacterial pathogen or a parasite.

### BRIEF DESCRIPTION OF THE DRAWINGS

The disclosure may be better understood by reference to one or more of these figures in combination with the detailed description of specific embodiments presented herein.

FIG. 1: Characterization of LDH nanoparticles. A. Representative TEM image of the LDH30 nanoparticles. B. Representative AFM image of the LDH30 nanoparticles. C. Representative TEM image of lysozyme-coated LDH30 nanoparticles. D. Particle size distribution of LDH30 and lysozyme-coated LDH30 (Lys@LDH30) based on DLS analysis. E. Particle size distribution of LDH30 and lysozyme-coated LDH30 (Lys@LDH30) based on TEM images. F. Thickness distribution of LDH30 nanoparticles based on AFM imaging.

FIG. 2: FTIR spectra of samples LDH30, lysozyme and lysozyme-coated LDH30 (Lys@LDH30).

FIG. 3: TEM images of lysozyme-coated LDH30 nanoparticles and LDH30 nanoparticle conjugated with mRNA. (A) Lys@LDH30; (B) LDH30-mRNA. The white arrow indicates mRNA strands conjugated with LDH30 stained with phosphotungstic acid.

FIG. 4: Nanoparticles aggregation caused by plant root exudates and mucilage and reduction in aggregation by addition of 20 mM KCl and lysozyme-coating of nanoparticles. A. Congo red-labelled, uncoated LDH nanoparticle aggregates on A. thaliana root surfaces after 12 h incubation with 200 mg/L Congo red-labelled LDH nanoparticles. B. Alleviation of aggregation of fluorescein-labelled LDH nanoparticles (LDH30-FL) in culture media by the addition of 20 mM KCl (KCl+), lysozyme or bovine serum albumin (BSA) coating of the nanoparticles (Protein+) but not by the addition of 20 mM MgCl₂ (MgCl+). Orange/red clusters in the suspension caused by aggregated particles.

FIG. 5: Fluorescein emit stronger fluorescence in alkaline environment (attached on LDH, pH ~10.5) than free in culture media (pH 6.1) or mild acidic root apoplast. A. Emission spectra of fluorescein under different pH excited at 480 nm. B. Images of fluorescein emission under different pH, excited with blue light hand lamps.

FIG. 6: Uptake and internalization of LDH nanoparticles in N. benthamiana roots. A. Representative confocal microscope images of transgenic N. benthamiana roots expressing RFP:ER after incubation with (i) 200 mg/L of Lys@LDH30-FL (labelled with 10 mg/L fluorescein) or (ii) 10 mg/L of free fluorescein, for 4 h. B. Quantitative analysis of co-localization ratio of RFP:ER and fluorescein in roots incubated with free fluorescein and Lys@LDH30-FL. C. Representative confocal images of roots after incubation with 200 mg/L of uncoated LDH30 nanoparticles loaded with fluorescein (LDH30-FL), BSA-coated LDH30 nanoparticles loaded with fluorescein (BSA@LDH30-FL), heat treated lysozyme-coated LDH30 nanoparticles loaded with fluorescein (HT-Lys@LDH30-FL) and lysozyme-coated LDH30 nanoparticles loaded with fluorescein (Lys@LDH30-FL), for 4 h. D. Quantitative fluorescence intensity of roots after incubation with free fluorescein, LDH30-FL and BSA@LDH30-FL, HT-Lys@LDH30-FL, and Lys@LDH30-FL for 4 h, normalized against the average intensity of the mature root of Lys@LDH30-FL treated group for comparison. B and D, Different lower-case letters above each bar represent a statistical difference of p<0.05 based on one-way ANOVA and post-hoc Tukey's analysis. Tip and mature root sections were analyzed separately. Data are presented as the mean±SEM, diamonds in the graph stands for actual values for each biological replicates.

FIG. 7: Representative confocal images demonstrating cellular internalization of lysozyme-coated LDH30 nanoparticles (Lys@LDH30-FL) in N. benthamiana RFP:ER roots at 4 h post incubation with 200 mg/L of Lys@LDH30-FL nanoparticles.

FIG. 8: Representative confocal images demonstrating uptake of lysozyme-coated LDH30 nanoparticles (Lys@LDH30-FL) in the zone of cell elongation near the root tip of N. benthamiana RFP:ER roots at 4 h post incubation with 200 mg/L of Lys@LDH30-FL nanoparticles.

FIG. 9: Enhanced uptake of LDH nanoparticles by N. benthamiana roots and tomato pollen following pretreatment with lysozyme. A. Representative confocal images of N. benthamiana roots uptake of LDH50-FL with/without lysozyme pretreatment. B. Representative confocal images of young bicellular pollen internalization of LDH50-FL (LDH nanosheets<50 nm in diameter and loaded with fluorescein) with and without lysozyme pretreatment. C. Flow cytometry results of young bicellular pollen internalization of LDH30-FL and LDH50-FL with/without lysozyme pretreatment. The fluorescence intensity was normalized based on the auto-fluorescence of pollen in blank control group. Different lower-case letters indicate significant differences at p<0.05 based on one-way ANOVA with post-hoc Tukey's analysis.

FIG. 10: A. Representative confocal images showing the effect of lysozyme pre-treatment on the uptake of uncoated LDH30 nanoparticles loaded with fluorescein by N. benthamiana roots and early bicellular pollen of tomato. B. Flow cytometry results showing enhanced uptake of LDH30-FL by early bicellular pollen after lysozyme pretreatment or coating. C. Flow cytometry results showing pretreatment or coating with high temperature treated lysozyme (HT-Lysozyme/HT-Lys) cannot effectively enhance the pollen uptake LDH30-FL. The fluorescence intensity was normalized based on the auto-fluorescence of pollen in blank control group. Different lower-case letters indicate significant differences at p<0.05 based on one-way ANOVA with post-hoc Tukey's analysis. Data are presented as the mean±SEM for n=3 biological replicates.

FIG. 11: Time-course confocal images of internalization of Lys@LDH30-FL by N. benthamiana mature roots within 4 h post incubation at 200 mg/L of Lys@LDH30-FL.

FIG. 12: Effect of Wortmannin, Brefeldin-A and low temperature treatment on lysozyme-coated LDH30 nanoparticle uptake and distribution in N. benthamiana roots. A. Representative confocal images of the root tip and epidermal layers of mature roots after incubation with Lys@LDH30-FL after a 4 h pre-treatment with Wortmannin (50 mM), Brefeldin-A (100 mM) and low temperature (4 °C). B. Quantitative fluorescence intensity of roots after incubation with Lys@LDH30-FL following pre-treatment with inhibitors and low temperature. The fluorescence intensity was normalized against the average intensity of control mature roots that were not pre-treated before incubation with Lys@LDH30-FL Lys@LDH30-FL (No inhibitor). Data presented as Mean±SEM. Different lower-case letters above each bar represented the statistical difference of p<0.05 by One-way ANOVA. The fluorescence intensity of tips and mature roots was analyzed separately, and n=3 for both tips and mature roots. The majority of the fluorescence signal observed and measured in the root in panels A and B of the figure was derived from the epidermal cells. C. Representative confocal microscope images of distribution of fluorescence in N. benthamiana roots after treatment with Lys@LDH30-FL nanoparticles and the exocytosis inhibitor Brefeldin-A. The focus of the images in panel C is through the centre of the root.

FIG. 13: RT-qPCR analysis of mRNA levels for genes encoding components of the membrane trafficking pathway in Arabidopsis roots after incubation with LDH30 and Lys@LDH. *** p=0.0040 (ESPIN1), p=0.0018 (VPS60.1); ** p=0.0174 (VPS36).

FIG. 14: Schematic illustration the apolplastic and membrane trafficking pathway involved in nanoparticle translocation through the root, involving repeated cycles of (1) apoplastic transport through the cell wall matrix, (2) endocytosis, (3) intracellular vesicular transport, and (4) exocytosis.

FIG. 15: Uptake and translocation of lysozyme-coated LDH30 nanoparticles by roots. A. Representative image of an N. benthamiana seedling at 4 h post incubation with lysozyme-coated LDH30 nanoparticles loaded with fluoroscein (Lys@LDH30-FL). Circled areas and letters (B-D, G and I) indicate the panel containing the corresponding confocal microscope images. B-F. Representative confocal images showing high accumulation of lysozyme-coated LDH30 loaded with fluorescein in the root tip (B), crack around the emerging lateral root (C), crack around the mature lateral root (D), root hairs (E and F), vasculature of petiole near the leaf base (I, row of images), at 4 h post incubation with Lys@LDH30-FL. G. Representative confocal images showing lysozyme-coated LDH30 nanoparticles loaded with fluorescein translocating through the stem of the seedling at 4 h post treatment (row of images). H. Zoomed in confocal image showing lysozyme-coated LDH30 nanoparticles loaded with fluorescein in the vasculature bundles of stem.

FIG. 16: pH-sensitive Lysosensor Green staining to indicate more acidic area of N. benthamiana root tips. The brighter green areas have a lower pH in the apoplast of elongating cells.

FIG. 17: Confocal microscope imaging showing colocalization of lysozyme-coated LDH30 nanoparticles labelled with fluorescein (Lys@LDH30-FL) with FM4-64 stained vesicles in a root hair of N. benthamiana.

FIG. 18: Electrophoresis image of biomolecules loading onto LDH30. A, Loading of mRNA at LDH:mRNA mass ratio 3:1. B, Loading of dsRNA at LDH:dsRNA mass ratio 7:1. C, Loading of plasmid DNA (pDNA) LDH at pDNA:LDH mass ratio of 5:1. The biomolecules completely complexed with LDH does not migrate to the bottom.

FIG. 19: Schematic illustration of hexangonal LDH nanoparticles being loaded with nucleic acids and then coated with lysozyme.

FIG. 20: A. Representative AFM images of LDH30 nanoparticle loaded with mRNA. B. Thickness distribution of LDH30 nanoparticles loaded with mRNA based on AFM imaging.

FIG. 21: Lysozyme-coated LDH30-mediated delivery of functional GFP mRNA to roots of several plant species. A. Representative confocal images of GFP expression in non-transgenic N. benthamiana roots at 2 days post incubation with naked mRNA or Lys@LDH30-mRNA at a mRNA concentration of 10 mg/L. B. Representative confocal images of GFP expression in A. thaliana, tomato (S. lycopersicum) and sorghum (S. bicolor) at 2 days post incubation with mRNA or Lys@LDH30-mRNA. C. Quantitative analysis of GFP fluorescence intensity relative to background florescence for non-transgenic N. benthamiana roots after incubation with Lys@LDH30-mRNA for 2 days. The root tip and mature root was analyzed separately. D. Quantitative GFP fluorescence intensity of non-transgenic N. benthamiana mature roots after incubation with Lys@LDH-mRNA with the LDH size of 30, 50, 80 and 120 nm. E. Time-dependent changes of the quantitative GFP fluorescence intensity of non-transgenic N. benthamiana mature roots after incubation with Lys@LDH30-mRNA. The mRNA concentration was 10 mg/L in all experiments. C-E, Data are presented as mean±SEM. Different lower-case letters indicate significance different with p<0.05 based on one-way ANOVA. Diamonds indicate for the value of individual replicate.

FIG. 22: Lysozyme-coated LDH30 nanoparticle delivery of GFP mRNA in N. benthamiana roots. A, Representative confocal images showing high GFP fluorescence in the root tip and zone of cell elongation and in lateral root junctions 2 days post incubated with Lys@LDH-mRNA at a mRNA concentration 10 mg/L. B, Confocal images of GFP translation for roots 2 days post incubated with 10 mg/L naked mRNA.

FIG. 23: Quantitative analysis of the GFP fluorescence intensity of Arabidopsis thaliana, tomato and sorghum roots at 2 days post incubation with naked mRNA and Lys@LDH-mRNA nanoparticles (mRNA concentration of 10 mg/L). All intensities were normalized against the autofluorescence of the blank group for each plant species. Different lower-case letters above the bars indicate significant difference at p<0.05 based on one-way ANOVA with post-hoc Tukey's analysis.

FIG. 24: Electrophoresis gel image showing that lysozyme complexes with mRNA at the higher protein:mRNA mass ratios of 20:1 and 30:1.

FIG. 25: Representative confocal images showing GFP translation in epidermal cells of the zone of cell elongation near the root tip and in cortical cells near lateral root junctions at 2 days post incubation of N. benthamiana roots with Lys@LDH120-mRNA.

FIG. 26: Representative confocal images of N. benthamiana roots showing internalization of 200 mg/L of Lys@LDH120-FL in the root tip and around the emerging site of a lateral root.

FIG. 27: Lysozyme-coated LDH30-mediated delivery of functional GFP mRNA into the roots and/or callus of several plant species. A. Representative confocal images of GFP expression in root of Arabidopsis, tomato and sorghum, and callus of sorghum at 2 days post incubation with naked mRNA or Lys@LDH30-mRNA. Dashed line circled the root outlines. B. Quantitative analysis of GFP fluorescence intensity analysis based on camara images of sorghum callus 2 days post treatment with lysozyme-coated LDH30 loaded with GFP mRNA (Lys@LDH30-mRNA) at a mRNA concentration of 5 mg/L. C. Camera images of sorghum callus 2 days post treatment with lysozyme-coated LDH30 loaded with GFP mRNA (Lys@LDH30-mRNA) at an mRNA concentration of 5 mg/L.

FIG. 28: Lysozyme-coated LDH30 nanoparticle delivery of functional dsRNA, siRNA and plasmid DNA into plant roots. A and B. Representative confocal microscope images and quantitative GFP fluorescence intensity of transgenic line N. benthamiana 16c roots at 3 days post incubation with dsRNA and Lys@LDH30-dsRNA (dsRNA concentration: 10 mg/L). C and D. Representative confocal microscope images and quantitative GFP fluorescence intensity of transgenic line N. benthamiana 16c roots at 3 days post incubation with siRNA and Lys@LDH30-siRNA (siRNA concentration: 5 mg/L). E and F. Representative confocal images of YFP expression in N. benthamiana roots and the quantitative YFP fluorescence intensity at 2 days post incubated with naked YFP coded plasmid DNA (pDNA) and Lys@LDH-pDNA (pDNA concentration=5 mg/L). B, D, F, Data presented as the mean±SEM. Different lower-case letters indicate significance different with p<0.05 based on one-way ANOVA, mature root and root tip were analyzed separately. Diamonds stand for the value of individual biological replicates (n=3).

FIG. 29: Lysozyme-coated LDH30 nanoparticle delivery of functional siRNA and plasmid DNA into roots. A, Representative confocal microscope images of N. benthamiana 16c mature roots at 3 days post incubation with siRNA and Lys@LDH-siRNA at siRNA concentration of 10 mg/L. B, Quantitative GFP fluorescence intensity at 2 days post incubated with naked plasmid DNA encoding a p35S:GFP transgene (pDNA-2) and lysozyme-coated LDH30 loaded with pDNA-2 (Lys@LDH-pDNA-2) at a DNA concentration of 5 mg/L. Different lower-case letters above the bars indicate significant difference at p<0.05 based on one-way ANOVA.

FIG. 30: Schematic map of short version (6132 bp) of plasmid DNA encoding a 35S:GFP transgene (pDNA-1).

FIG. 31: Schematic map of long version (14699 bp) of plasmid DNA encoding a 35S:GFP transgene (pDNA-2).

FIG. 32: Lysozyme-coated LDH30 nanoparticle delivery of functional dsRNA, siRNA and/or plasmid DNA into roots. A-D. Representative confocal images of GFP expression in N. benthamiana roots (A) and the quantitative GFP fluorescence intensity (C) at 2 days post incubated with naked short plasmid DNA encoding 35S:GFP transgene (pDNA-1) and lysozyme-coated LDH30 loaded with pDNA-1 or pDNA-2 (Lys@LDH-pDNA-1 and -2) at a DNA concentration of 10 mg/L. Representative confocal images of GFP expression in Sorghum bicolor roots (B) and the quantitative GFP fluorescence intensity (D) at 2 days post incubated with naked plasmid DNA encoding a pUbi:GFP transgene (pDNA-2) and lysozyme-coated LDH30 loaded with pDNA-2 (Lys@LDH-pDNA-2) at a DNA concentration of 5 mg/L.

FIG. 33: Representative confocal microscope images of Nicotiana benthamiana root 2 days post incubation with 14.7 kb 35S:GFP plasmid (Lys@LDH30-pDNA-2) at a pDNA concentration of 10 mg/L.

FIG. 34: Camera images of Nicotiana benthamiana root incubated with lysozyme-coated LDH30 loaded with 6 kb 35S:GFP plasmid (Lys@LDH30-pDNA-1) and 14.7 kb 35S:GFP plasmid (Lys@LDH30-pDNA-2) at a pDNA concentration of 10 mg/L.

FIG. 35: Schematic map of plasmid DNA encoding a UBI:GFP transgene (pDNA-3).

FIG. 36: Lysozyme-coated LDH30 nanoparticle delivery of functional plasmid DNA into leaves and pollen. A. Representative confocal microscope images of N. benthamiana leaf mesophyll and epidermal cells 3 days post infiltration with lysozyme-coated LDH30 loaded with plasmid DNA encoding a 35S.GFP transgene (Lys@LDH30-pDNA-3) at a DNA concentration of 20 mg/L. B-C. Representative confocal images (B), flow-cytometer histogram GFP-positive pollen ratio (C) of pollen extracted 3 days post injection of flower buds with Lys@LDH30-pDNA-3 at pDNA concentration of 20 mg/L. Quantitative GFP fluorescence intensity is presented as the mean±SEM of n=3 biological replicates. Different lower-case letters indicate a significance different of p<0.05 based on one-way ANOVA; mature root and root tip were analyzed separately. K, Data presented are the mean±SEM for n=4 biological replicates, ** p=0.0040. Diamonds stand for the value of individual biological replicates.

FIG. 37: Flow cytometer results showing pollen count plotted against fluorescent intensity of extracted pollen from tomato flower buds 3 days post injection of Lys@LDH30-pDNA-3 at pDNA concentration of 20 mg/L.

FIG. 38: LDH50 nanoparticles facilitate dsRNA and siRNA uptake, and the induction of RNAi. A. TEM image of LDH50 (<50 nm in diameter) nanoparticles. B. Early bicellular tomato pollen showing the vegetative nucleus (VN) and generative cell (GC). C. FITC-labelled LDH50 nanoparticles but not larger LDH120 nanoparticles (120 nm in diameter), are internalised by early bicellular tomato pollen. D. GUS mRNA levels in GUS-expressing early bicellular tomato pollen following a 3 day incubation in 20 μg/mL GUS dsRNA ((GUS) dsRNA) alone or in complex with LDH50 (LDH-dsRNA). E. Petiole infiltration of excised tobacco leaf with FITC-labelled LDH50 v. F. Translocation of FITC-labelled LDH50 nanoparticles along the vasculature of an infiltrated excised tobacco leaf; inset shows higher magnification of a leaf vein. G. GFP mRNA levels in mesophyll tissue of GFP-expressing tobacco leaves at 1-day post-infiltration with a 21 nt GFP siRNA duplex alone ((GFP) siRNA) or in complex with LDH50 nanoparticles (LDH-siRNA; 10 μg/mL).

FIG. 39: Uptake and translocation of lysozyme-coated LDH50 nanoparticles in whole seedlings and excised shoots, and functionality of delivered mRNA and plasmid DNA. A. Tobacco root 4 h after incubation in fluorescein-labelled LDH50 (200 μg/mL). B-C. Enhanced uptake of lysozyme-encapsulated fluorescein-labelled LDH50 (200 μg/mL) into a tobacco root (B) and whole seedling (C) 4 h after treatment. D. Mature root and (E) leaf petiole images of the seedling in (C) showing nanoparticles in the vasculature (white arrows). Red, chlorophyll florescence. F-H. A 4 h pre-treatment with the endocytosis inhibitor Wortmannin (F) or cold (G) completely blocked uptake of lysozyme-encapsulated LDH50, and the exocytosis inhibitor Brefeldin-A (H) prevented their translocation from the root tip to other parts of the root (white line represents root outline; compare images to B). I. Arabidopsis leaf primordium (<0.5 mm in diam.) and J. developing leaf (2-5 mm in length) 10 h after incubation of excised shoot in media containing lysozyme-coated FITC-labelled LDH50 (200 μg/mL); shoots were excised 1-2 mm below the cotyledons and shoot apex. Nanoparticles were detected in the vasculature (white arrows) and surrounding cells (I-J). K. Tobacco root showing GFP florescence 2 days after incubation in media containing GFP mRNA (10 μg/mL) in complex with uncoated LDH50. L-M. Tobacco root showing GFP florescence 2 days after incubation in media containing GFP mRNA (L; 10 μg/mL) or plasmid DNA encoding an intron-spliceable 35S:GFP transgene (M; 5 μg/mL) in complex with lysozyme-coated LDH50 nanoparticles. Bar indicates 100 μm, except in panel C.

FIG. 40: Lysozyme-coated LDH delivery of PCR-amplified transgene induces higher expression of the transgene relative the expression of the transgene when delivered via a supercoiled or linearized plasmid. A. Schematic representation of p35S:GFP transgene fragment comprising, in a 5′ to 3′ direction, the p35S promoter, the eGFP transgene and ocs-element. B. GFP intensity observed in root tips and mature roots of N. benthamiana delivered lysozyme-coated LDH30 nanoparticles loaded with nothing (untreated Control), PCR-amplified p35S:GFP transgene (p35S:GFP), supercoiled pDNA-1 plasmid (S-p35S:GFP) and linearised pDNA-1 plasmid (L-p35S:GFP), respectively.

FIG. 41: Confocal microscope images showing p35S:GFP expression in mature root (upper and lower) and root tips of Arabidopsis seedlings incubated with Lys@LDH30-pDNA or NLS@LDH30-pDNA.

FIG. 42: Confocal microscope images showing p35S:GFP expression throughout the root tissue of Arabidopsis seedlings incubated with Lys@LDH30-pDNA or NLS@LDH30-pDNA.

FIG. 43: Quantitative analysis of GFP fluorescence intensity relative to background florescence for non-transgenic Arabidopsis roots after incubation of roots for 3 days with lysozyme-coated LDH30 loaded with pDNA-1 (Lys@LDH30-pDNA), NLS-coated LDH30 loaded with pDNA encoding p35S:GFP (NLS@LDH30-pDNA), NLS and Lysozyme co-coated LDH30 loaded with pDNA-1 (NLS&Lys@LDH30-pDNA) and NLS-coated LDH30 loaded with pDNA and pre-treated with lysozyme (NLS@LDH30-pDNA (Lys pre-treatment)). The (A) mature root and (B) root tips were analyzed separately. Students t-test: *, p<0.0001; **, p=0.0012; *, p=0.0218.

FIG. 44: Confocal microscope images showing GFP expression in root tips, mature root, stem, and leaf of Control Arabidopsis seedlings and Arabidopsis seedlings treated with Lys@LDH30-pDNA encoding p35S:GFP (“GFP”) or Lys@LDH30-pDNA-1mod (“GFP: tRNA).

### DETAILED DESCRIPTION

### General Techniques and Definitions

Unless specifically defined otherwise, all technical and scientific terms used herein shall be taken to have the same meaning as commonly understood by one of ordinary skill in the art (e.g., in genomics, immunology, molecular biology, immunohistochemistry, biochemistry, plant breeding and plant biology).

The present disclosure is performed without undue experimentation using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition., 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984), and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein. Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

The term “about” is used herein to mean approximately. When the term “about” is used in conjunction with a numerical range, it modifies that range by extending the boundaries above and below the recited numerical values. In general, the term “about” is used herein to modify a numerical value, such as an amount of time, concentration, temperature etc., above and below the stated value by +10%, more preferably +5%, even more preferably +1%, and still more preferably +0.1% from the specified value as appropriate to perform the disclosed method.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### Compositions

As described herein, the inventors have shown that small LDH nanoparticles coated in polypeptide or peptide (collectively referred to as “protein”) having an overall positive surface charge can be used effectively to deliver functional forms of nucleic acids (e.g., RNA and DNA) into plant tissue. In particular, the inventors have demonstrated experimentally that lysozyme- or Nuclear Localisation Signal (NLS) peptide-coating of the LDH nanoparticles greatly enhanced this process due to the protein-mediated engagement in the endocytosis and the membrane trafficking pathway of plants to facilitate the efficient uptake and translocation of the LDH nanoparticles throughout the plant tissues. Specifically, it was shown that both lysozyme- and NLS-coated LDH nanoparticles exhibited reduced aggregation on the surface of the plant tissue and in culture media relative to non-coated LDH nanoparticles, thereby resulting in improved uptake and cell-to-cell translocation of the coated nanoparticles. Without being bound by any one theory, the inventors also believe that the improved uptake and cell-to-cell translocation of the nanoparticles is, in part, due to the ability of the protein-coated LDH nanoparticles to access the membrane trafficking pathway of plants. It is also believed that the lysozyme-coated nanoparticles enhanced the internalization and translocation of the LDH nanoparticles by degrading polysaccharides in the plant cell wall, allowing the nanoparticles to diffuse more freely through the apoplast (i.e., the plant cell wall matrix) and readily engage in endocytosis via the cell membrane. Based on this finding, the inventors have developed a highly efficient, non-GM, LDH-based nanodelivery system that can be used universally to deliver various forms of functional nucleic acids or exogenous biomolecules to plant tissue in order to improve biological functions in plant and protect plants both biotic and abiotic stresses, regardless of species.

Accordingly, in one example, the present disclosure provides a composition comprising one or more LDH nanoparticles onto which one or more nucleic acid molecules are adsorbed and which are coated in protein having a positive surface charge.

Layered double hydroxide (LDH) is a class of compounds that contain two metal cations and have a layered structure. LDH is outlined in “Structure and Bonding” Vol 119 (2005) “Layered Double Hydroxides”. Hydrotalcite is the most well-known example of LDH and has been studied extensively. LDH can intercalate anions between structural layers. LDHs are mixed hydroxides of divalent and divalent metals having an excess of positive charge that is balanced by interlayer anions. Common forms of LDH comprise Mg2+ and Al3+ (known as hydrotalcite) and Mg2+ and Fe3+ (known as pyroaurite) but LDHs containing other cations including Ni, Zn, Mn, Ca, Cr, and La are known. The LDH may also be of the quintinite group, the fougerite group, the woodwardite group, the cualstibite group, the glaueocerinite group, the wermlandite group, and the hydrocalumite group. The amount of surface positive charge generated is dependent upon the mole ratio of the metal ions in the lattice structure, and the conditions of preparation as they affect crystal formation. Exemplary LDH, and methods of making LDH, are described in WO2006/066341, the contents of which are incorporated herein by reference. Advantageously, in the method described herein, the size of the LDH can be precisely controlled and the hydrothermal treatment can disperse the LDH agglomerates into individual LDH nanoparticles. The method of synthesizing LDH nanoparticles can be performed by any method known to the skilled person. For example, the LDH nanoparticles can be synthesized according to the method described in Yong, J., et al., Sheet-like clay nanoparticles deliver RNA into developing pollen to efficiently silence a target gene. Plant Physiol, 2021. 187 (2), pages 896 to 898. Further modifications to the method is described in Example 1 herein.

The skilled person will appreciate that the number or concentration of LDH nanoparticles in the composition may be adjusted to suit the particular plant tissue and application. For example, the composition may comprise the LDH nanoparticles at a concentration of about 0.0001 g/L to about 50 g/L. For example, the composition may comprise the LDH nanoparticles at a concentration of about 0.001 g/L to about 50 g/L. For example, the composition may comprise the LDH nanoparticles at a concentration of about 0.01 g/L to about 40 g/L. For example, the composition may comprise the LDH nanoparticles at a concentration of about 0.1 g/L to about 30 g/L. For example, the composition may comprise the LDH nanoparticles at a concentration of about 1 g/L to about 20 g/L. In accordance with one example in which the composition is added to, or diluted in, plant tissue culture medium, the tissue culture medium may contain the LDH nanoparticles at a concentration of about at 0.001-0.1 g/L and the nucleic acids adsorbed thereto at a concentration of about 0.0005-0.020 g/L. However, the skilled person will appreciate that the concentration of the LDH nanoparticles and the concentration of the nucleic acids adsorbed thereto may be varied according to the application.

The term “protein” as used herein refers to a polypeptide (i.e., a string of amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a fragment or portion thereof. A non-exhaustive list of the types of proteins include antibodies, contractile proteins, enzymes, hormonal proteins, structural proteins, storage proteins, and transport proteins. Furthermore, for the sake of convenience, the term “protein” as used in the context of “proteins having an overall positive surface charge” shall also be understood to include peptides (i.e., a short string of amino acids linked to one another by peptide bonds, preferably at least 6 amino acids in length).

The terms “positive surface charge” or “overall positive surface charge” and similar, as used in the context of a protein, shall be understood to refer to a positive electrostatic potential on a protein's surface. This surface charge may be influenced by, inter alia, the charge of those amino acids which make up the protein, as well as the charge of those amino acids which are exposed on the protein's surface when correctly folded. The charge of a protein may also be influenced by the pH of their environment. Accordingly, in one example, the phrase “a protein having a positive surface charge” or “protein having an overall positive surface charge” refers to a protein whose net surface charge is positive when correctly folded at a given pH. For example, the protein having an overall positive surface charge as described herein may have net surface charge which is positive when correctly folded at a pH between about 4 and about 10. For example, the protein having an overall positive surface charge as described herein may have net surface charge which is positive when correctly folded at a pH between about 5 and about 7. For example, the protein having an overall positive surface charge as described herein may have net surface charge which is positive when correctly folded at a pH between about 6 and about 7. In other examples, the protein having an overall positive surface charge as described herein may have net surface charge which is positive when correctly folded at a pH between about 7 and about 8, or the protein having an overall positive surface charge as described herein may have net surface charge which is positive when correctly folded at a pH between about 8 and about 10

The surface charge density of a particle, however, may be influenced by a change in the size (and therefor surface area) of the particle. In the context of the LDH nanoparticles, coating of the LDH nanoparticles with the protein can increase or decrease the positive charge density of the nanoparticle's surface, depending on the extent that the protein coating changes the overall size of the nanoparticle. For example, uncoated LDH has a surface charge of 30-40 mV, whereas lysozyme- and NLS-coated LDH has a surface charge of ~20 and ~70 mV, respectively.

In accordance with some examples, the protein having a positive surface charge is capable of degrading polysaccharides in a plant cell wall. The plant cell wall is a rigid, semi-permeable structure comprised mainly of polysaccharides with some proteins and lipids. The three main polysaccharide components of the cell wall are cellulose, an unbranched polymers of β-(1-4)-D-glycopyranosyl units associated in microfibril bundles. The microfibrils are cross-linked by hemicellulose (a branched polymer of β-(1-4)-D-xylopyranosyl units). This cross-linked structure is embedded in a matrix of pectin (primarily containing an α-(1-4) polygalacturonic acid backbone which can be randomly acetylated and methylated. As used herein, the term “polysaccharide” refers to polymers comprising a backbone consisting mainly (at least about 90%) of monosaccharide repeating units and/or derivatized monosaccharide repeating units. In one example, plant cell wall polysaccharides include, but are not limited to, cellulose, hemicellulose, and pectic substances. Accordingly, in some examples, a protein having a positive surface charge which can be used in the composition and method of the disclosure may be capable of degrading those polysaccharides component present in the plant cell wall.

The ability of a protein to degrade polysaccharides in the plant cell wall may be, for example, as a result of enzymatic activity of the protein. Accordingly, in one example, the protein capable of degrading polysaccharides in a plant cell wall may be an enzyme. In one example, the protein is a polysaccharide-degrading enzyme. As used herein, the term “polysaccharide-degrading enzyme” refers to any enzyme that can be utilized to promote the degradation of the plant cell wall polysaccharides into fermentable sugars. Such enzymes are known in the art and include, but are not limited to, enzymes that can catalyze the degradation of cellulose, hemicellulose, and/or pectin. In further examples, the enzyme is a lysozyme. In other examples, the lysozyme is obtained from chicken egg white. The enzymes and genes thereof referenced herein may be obtained from any suitable origin, including, bacterial, fungal, yeast or mammalian origin.

In one particular example, the protein capable of degrading polysaccharides in a plant cell wall is a lysozyme. As used herein, the term “lysozyme” is a hydrolytic glycosidase that is capable of hydrolyzing the glycosidic linkages in polysaccharides and thereby degrading a plant cell wall. Lysozymes may exist as all naturally-occurring lysozymes, such as hen egg white lysozyme, synthetic lysozymes and recombinant lysozymes, such as human recombinant lysozyme, as well as lysozyme salts.

In another example, the protein having an overall positive surface charge is, or comprises, a nuclear localization signal (NLS) peptide. As used herein, the term “Nuclear Localization Signal (NLS) peptide”, “Nuclear Localization Sequence (NLS) peptide”, “NLS peptide” or similar, refer to a class of generally short and highly conserved peptides that act as a signal fragment to mediate transport of proteins from the cytoplasm to the nucleus of cells. Many NLS peptides are known in the art and are contemplated for use herein, provided that they possess the quality of having an overall positive surface charge. In some examples, the protein having an overall positive surface charge may be, or comprise, a NLS peptide derived from a Simian virus 40 (SV40) T antigen. For example, the protein having an overall positive surface charge may comprise or consist of the amino acid sequence set forth in SEQ ID NO: 5.

As described herein, the one or more LDH nanoparticles are coated with the protein having a positive surface charge. Coating of the one or more LDH nanoparticles with the protein having a positive surface charge provides an overall positive surface charge to the LDH nanoparticles. Coating of the LDH nanoparticles with the protein may be performed using any method known in the art. Suitably, the protein may be coated onto the one or more LDH nanoparticle through electrostatic interactions following adsorption of one or more nucleic acids molecules onto a LDH nanoparticle. In this way, the protein may encapsulate the LDH nanoparticle and the nucleic acid molecules adsorbed thereto. As used herein, the term “electrostatic interaction” refers to an intermolecular interaction between two or more positively or negatively charged moieties/groups, which may be attractive when two are oppositely charged (i.e., one positive, another negative), repulsive when two charges are of the same sign (i.e., two positive or two negative), or a combination thereof. Electrostatic interaction can be modulated by including positively and negatively charged moieties/groups on the surface of the nanoparticles. However, any method known in the art for coating nanoparticles with protein is contemplated for use herein.

In some examples, the one or more LDH nanoparticles are coated with the protein at a mass ratio of about 1:2 (LDH nanoparticle:protein) to about 1:20 (LDH nanoparticle:protein). In other examples, the one or more LDH nanoparticles are coated with a protein at a mass ratio of about 1:5 to about 1:15, or about 1:4 to about 1:8, or about 1:4 to about 1:6. In further examples, the one or more LDH nanoparticles are coated with a protein at a mass ratio of about 1:3, about 1:4, about 1:5, about 1:6, about 1:7, about 1:8, about 1:9 or about 1:10.

The dimensions of the protein-coated LDH nanoparticles may be varied as required. In some examples, the LDH nanoparticles have an average diameter in the range of about 15 to 400 nm, or about 20 to 300 nm, or about 25-200 nm, or about 30 to 100 nm, or about 30 to 50 nm. In one particular example, the one or more LDH nanoparticles have an average diameter in the range of about 30 nm to about 50 nm. The average particle size may be determined using any method known in the art. In one example, the average particle size is determined using transmission electron microscopy. However, other light scattering methods (e.g., laser diffraction) can be used as well. The term “average diameter” as used in the context of LDH nanoparticle size refers to the average diameter of non-agglomerated and/or non-aggregated single nanoparticles at their widest point. Alternatively, or in addition, the nanoparticles may exhibit a narrow particle size distribution, and the particles may show a particle size distribution of =20% around the average size. The LDH nanoparticles may have an aspect ratio that, falls within the range of from 5 to 15 (the ‘aspect ratio’ relates to the ratio of the largest dimension of the nanoparticle to its thickness or height). In one example, the ratio is about 6:12, or about 7:10, In some examples, the ratio is about 8:9.

As described herein, one or more nucleic acid molecules are attached to the surface of the LDH nanoparticle by virtue of being adsorbed to the protein coating the surface of the LDH nanoparticle. As used herein, the term “nucleic acid” refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G. Tor U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, at least one of the nucleic acid molecules which is attached to the surface of the LDH nanoparticle is a deoxyribonucleic acid (DNA) molecule or derivatives thereof. In another example, at least one of the nucleic acid molecules which is attached to the surface of the LDH nanoparticle is a ribonucleic acid (RNA) molecule, or derivatives thereof. A nucleic acid molecule(s) may be single-stranded or double stranded.

In one example, the one or more nucleic acid molecules attached to the surface of the LDH nanoparticle encode a protein or polypeptide of interest or a functional fragment thereof. In accordance with this example, the one or more nucleic acid molecules may be messenger RNA (mRNA), non-coding RNA, PCR-amplified DNA, or plasmid DNA (pDNA) molecules comprising a polynucleotide sequence encoding the protein or polypeptide of interest or functional fragment thereof. In some examples, the polynucleotide sequence encoding the protein or polypeptide of interest, or functional fragment thereof, further comprises a mobile transfer RNA (tRNA), such as in the 3′ UTR, in order facilitate systematic expression of the protein or polypeptide of interest, or functional fragment thereof. In some examples, the polynucleotide sequence is a noncoding RNA, e.g., a synthetic or plasmid-encoded small guide RNA (sgRNA) for guiding gene editing, further comprises a mobile transfer RNA (tRNA), such as in the 3′ UTR, in order facilitate its systematic functioning. In this regard, mobile transfer RNA (tRNA)-derived sequences are able to mediate transport of protein-coding mRNA via the phloem vasculature, including through graft junctions (e.g., Zhang et al (2016) Plant Cell 28:1237-1249).

As used herein, the term “messenger RNA” or “mRNA” refers to a polyribonucleotide that encodes at least one polypeptide. mRNA as used herein encompasses both modified and unmodified mRNA. mRNA may contain one or more coding and non-coding regions and can be purified from natural sources, produced using recombinant expression systems and optionally purified, in vitro transcribed, or chemically synthesized.

As used herein, the term “plasmid DNA” or “pDNA” refers to a small DNA molecule that is typically circular and is capable of replicating independently. pDNAs are constructs comprised of genetic material designed to direct transformation of a targeted cell e.g., plant cells. The plasmid DNA may be supercoiled or linearized.

In other examples, the one or more nucleic acid molecules attached to the surface of the LDH nanoparticle are capable of modulating expression of one or more genes within a plant cell. For example, the one or more nucleic acid molecules may be RNA interference (RNAi) molecules, capable of post-transcriptional gene silencing or suppression, RNA silencing, and/or decreasing gene expression. Interfering RNAs affect sequence-specific, post-transcriptional gene silencing in animals and plants by base pairing to the mRNA sequence of a target nucleic acid. Techniques for the design of such molecules for use in targeted inhibition of gene expression are well known to one of skill in the art. Exemplary RNAi molecules include, but are not limited to, double stranded RNA (dsRNA), a short interfering RNA (siRNA), single stranded RNA (ssRNA) molecules, short hairpin RNA (shRNA) molecules, short microRNA (shmiR) molecules, primary miRNA transcripts (pri-miRNA) and precursor microRNA (pre-miRNA). Accordingly, in some examples, the one or more nucleic acid molecules may be RNAi molecules targeting mRNA transcripts associated with plant pathogens or pests. Other targets of RNAi molecules which are contemplated within the scope of the disclosure include RNAi molecules targeting mRNA transcripts corresponding to target genes which regulate agronomic traits of interest, such as, production of phenolic compounds, uniformity in flowering and maturity, days to anthesis, germination rate, grain weight or size (e.g., thousand seed weight), awn length, spike length, plant density, plant height, total biomass, oil content, fertilizer utilization and responsiveness, water usage, growth rate, for example. In yet another example, a nucleic acid molecule capable of modulating expression of one or more genes within a plant cell may be a microRNA (miRNA), a transfer RNA (tRNA), or a ribosomal RNA (rRNA) molecule. Accordingly, the one or more nucleic acid molecules attached to the surface of the LDH nanoparticles may be a miRNA, tRNA or rRNA.

As used herein, the term “double stranded RNA” or “dsRNA” refers to two complementary RNA molecules that have annealed to one-another to form a double stranded RNA molecule. The two strands can be of identical length or of different lengths provided there is enough sequence homology between the two strands that a double stranded structure is formed with at least 80%, 90%, 95% or 100% complementarity over the entire length.

When a dsRNA is provided as a hairpin or stem loop structure it can be referred to as a “hairpin RNA” or “short hairpin RNAi agent” or “shRNA”. Other dsRNA molecules provided in, or which give rise to, a hairpin or stem loop structure include primary miRNA transcripts (pri-miRNA) and precursor microRNA (pre-miRNA). Pre-miRNA shRNAs can be naturally produced from pri-miRNA by the action of the enzymes Drosha and Pasha which recognize and release regions of the primary miRNA transcript which form a stem-loop structure. Alternatively, the pri-miRNA transcript can be engineered to replace the natural stem-loop structure with an artificial/recombinant stem-loop structure. That is, an artificial/recombinant stem-loop structure may be inserted or cloned into a pri-miRNA backbone sequence which lacks its natural stem-loop structure. In the case of stemloop sequences engineered to be expressed as part of a pri-miRNA molecule, Drosha and Pasha recognize and release the artificial shRNA. dsRNA molecules produced using this approach are known as “shmiRNAs”, “shmiRs” or “microRNA framework shRNAs”.

As used herein, the term “small interfering RNA” or “siRNA” means double stranded ribonucleotide sequences of typically 15-50 base pairs and preferably 19-27 base pairs in length that are highly negatively charged and soluble predominantly in water. siRNA may be composed of either two annealed ribonucleotide sequences or a single ribonucleotide sequence that forms a hairpin structure.

As used herein, the term “single stranded RNA” or “ssRNA” refers to a single-stranded RNA molecule or oligoribonucleotide. A “ssRNA” may be a homopolymer (e.g., polyl, polyC or polyG) or may be a heterogeneous polymer composed of, for example, a random sequence of different ribonucleotides or simply two different ribonucleotides. In addition, ssRNA may comprise regions of secondary or tertiary structure.

As used herein, the term “transfer RNA” or “tRNA” refers to an RNA chain that transfers an amino acid to a growing polypeptide chain. The tRNA has sites for amino acid attachment and codon recognition.

As used herein, the term “microRNA” or “miRNA” or “precursor-microRNA” refers to a ribonucleotide molecule acting as a post-transcriptional regulator. MicroRNAs (miRNAs) are non-protein coding RNAs, generally of between about 19 to about 25 nucleotides (commonly about 20-24 nucleotides in plants). miRNAs direct cleavage in trans of target transcripts, regulating the expression of genes involved in various regulation and development pathways (Bartel, Cell, 116:281-297 (2004); Zhang et al. Dev. Biol. 289:3-16 (2006)). miRNAs have been shown to be involved in different aspects of plant growth and development as well as in signal transduction and protein degradation. In addition, growing evidence indicates that small endogenous mRNAs including miRNAs may also be involved in biotic stress responses such as parasite attack.

As used herein, the term “ribosomal RNA” or “rRNA” refers to the non-coding RNA which is the primary component of ribosomes. rRNA is a ribozyme which carries out protein synthesis in ribosomes.

The term “adsorbed” as used herein shall be understood to mean that the one or more nucleic acid molecules are immobilized or adhered to the surface of the LDH nanoparticles via non-covalent interactions. In accordance with examples in which the LDH nanoparticles are coated with a protein having a positive surface charge, the one or more nucleic acid molecules will be adsorbed to the protein coating.

The one or more nucleic acid may be adsorbed onto the protein-coated LDH nanoparticles in any suitable loading ratio. Exemplary loading ratios (by mass) of nucleic acid to LDH nanoparticles include from 2:1 to 1:20, or from 1:1 to 1:10, or from 1:1 to 1:6 or from 1:1 to 1:5 or from 1:1 to 1:4, or from 1:1 to 1:2.5, or from 1:2 to 1:5 or from 1:3 to 1:4. The loading ratio may be 1:1, 1:1.5, 1:2, 1:2.5, 1:3, 1:3.5 or 1:4.

The composition of the disclosure may be provided in any suitable form. For example, the composition may be in the form of a solid, ointment, gel, cream, powder, paste, suspension, colloid, foam or aerosol; especially a suspension or a colloid. Solid forms of the composition may include dusts, powders, granules, pellets, pills, pastilles, tablets, filled films (including seed coatings) and the like, which may be water-dispersible. In another example, the composition is in the form of a concentrate, optionally in the form of a colloid or suspension. In a further example, the composition is heterogeneous, comprising a solid phase dispersed within a fluid phase. The solid phase may comprise the one or more nucleic acid molecules adsorbed onto the protein-coated LDH nanoparticles. The fluid phase may be, for example, a liquid, a gas, or a free flowing solid, or a combination thereof. For example, a suitable liquid may be an aqueous liquid, such as water. The water may be sterile or non-sterile. The solid-phase may be dispersed within the fluid phase in any suitable way depending upon the nature of the solid-phase and the fluid-phase. Depending on the form of the composition, the composition may include a variety of other agents. Exemplary agents include, but are not limited to, one or more of the following types of ingredients: diluents, carriers, excipients, suspension agents, agglomeration agents, bases, buffers, bittering agents, fragrances, preservatives, propellants, thixotropic agents, anti-freezing agents, and coloring agents. Suitable agents may be selected by a skilled person. The composition may also include one or more other active ingredients. An active ingredient, as defined herein, is an ingredient that provides benefit to a plant. The active ingredient may be, for example, an insecticide, a pesticide, a fungicide, an antibiotic, an insect repellant, an anti-parasitic agent, an anti-viral agent, or a nematicide.

In some examples, the composition may be formulated for administration to the plant, or to any part of the plant, in any suitable way. For example, the composition may be formulated for administration to the leaves, stem, roots, fruit, vegetables, flowers, ovules, grains and/or pulses of the plant. In one example, the composition may be administered to the plant as a metered dose. In another example, the composition may be formulated for administration to the plant, for example, by spraying, by brush or by another applicator. In some examples, the composition is formulated for administration to the roots of the plant in the form of a suspension and is present in an aqueous media in which the plant roots are submerged.

### Methods and Uses

The present disclosure also provides a method of introducing one or more nucleic acid molecules to a plant tissue, comprising contacting the plant tissue with a protein having an overall positive surface charge and a LDH nanoparticle onto which the nucleic acid molecules are adsorbed.

In one particular example, the one or more nucleic acid molecules are introduced to the plant tissue in the form of a composition as described herein. That is, a composition comprising one or more LDH nanoparticles onto which the one or more nucleic acid molecules are adsorbed and coated in a protein having an overall positive surface charge.

However, in an alternative example, the method may comprise separately contacting the plant tissue with the protein having an overall positive surface charge and the LDH nanoparticles onto which the one or more nucleic acid molecules are adsorbed. In this regard, the inventors have shown that a number of the advantages associated with the protein-coated LDH nanoparticles in terms of reduced aggregation and improved uptake and cell-to-cell translocation of the nanoparticles (as described herein) can also be achieved when the protein and the LDH nanoparticles are contacted with the plant tissue separately. That is, when LDH nanoparticles onto which one or more nucleic acid molecules are adsorbed are contacted with the plant tissue in the presence of the protein having a positive surface charge. In this regard, a method of the disclosure contemplates contacting the plant tissue with the protein and the LDH nanoparticle simultaneously or consecutively. In some example, the plant tissue is contacted with the protein prior to being contacted with the one or more LDH nanoparticles. For example, the protein may be present in the incubation medium in which the plant tissue is immersed. In accordance with an example in which the plant tissue is contacted with the protein prior to being contacted with the one or more LDH nanoparticles i.e., protein pre-treatment, the protein pre-treatment may commence 15 mins or more prior to contacting the plant tissue with the one or more LDH nanoparticles. For example, the protein pre-treatment may commence about 30 min to about 12 hours (e.g., about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours) prior to contacting the plant tissue with the one or more LDH nanoparticles. In each of the foregoing examples describing protein pre-treatment, the plant tissue may be in contact with the protein (e.g., incubated or immersed) for 15 min or more. For example, the protein pre-treatment may comprise contacting the plant tissue with the protein for between about 30 min to about 6 hours (e.g., for about 1 hour, or for about 2 hours, or for about 3 hours, or for about 4 hours, or for about 5 hours, or for about 6 hours). In some examples, the plant tissue may be washed (e.g., with saline, water or fresh culture media) following the protein pre-treatment in order to remove any excess protein from the tissue prior to contacting the plant tissue with the one or more LDH nanoparticles.

In other examples, the method of introducing one or more nucleic acid molecules to a plant tissue comprises contacting the plant tissue with the protein having an overall positive surface charge and the one or more LDH nanoparticles onto which the one or more nucleic acid molecules are adsorbed simultaneously. For example, the one or more LDH nanoparticles may be coated with the protein and contacted with the plant tissue. Alternatively, the one or more LDH nanoparticles and the protein having an overall positive surface charge may be provided separately and contacted with the plant tissue simultaneously.

Exemplary proteins, LDH nanoparticles and nucleic acid molecules are described herein in the context of the compositions of the disclosure and shall be taken to apply mutatis mutandis to each and every example describing methods in which the LDH nanoparticles and the protein are contacted with plant tissue separately when introducing one or more nucleic acid molecules thereto.

As described herein in the context of the composition of the disclosure, the protein having an overall positive surface charge is capable of stimulating the endocytosis and/or membrane trafficking pathways in a plant or plant tissue. In some examples, the protein is capable of degrading polysaccharides in a plant cell wall, such as an enzyme. Exemplary enzymes which are capable of degrading polysaccharides are known in the art and described herein. However, in one particular example, the protein having an overall positive surface charge is lysozyme. In another example, the protein having an overall positive surface charge is, or comprises, a nuclear localization signal (NLS) peptide. For example, the protein having an overall positive surface charge may be, or comprise, a NLS peptide derived from a Simian virus 40 (SV40) T antigen. For example, the protein having an overall positive surface charge may comprise or consist of the amino acid sequence set forth in SEQ ID NO: 5.

Based on the inventors' finding that pre-treatment of plant tissue (e.g., roots) with lysozyme enhances the uptake and cellular internalisation of NLS-coated LDH nanoparticles but not lysosome-coated LDH nanoparticles, the present disclosure contemplates a method in which the plant tissue is contacted with lysozyme prior to being contacted with the one or more LDH nanoparticles i.e., lysozyme pre-treatment, and the one or more LDH nanoparticles are coated with a further protein having an overall positive surface charge e.g., as described herein, which is not lysozyme. In some examples, the further protein having an overall positive surface charge is, or comprises, a NLS peptide derived from a Simian virus 40 (SV40) T antigen. For example, the further protein having an overall positive surface charge may comprise or consist of the amino acid sequence set forth in SEQ ID NO: 5. In accordance with this particular embodiment, the lysozyme pre-treatment may be performed as described for the protein pre-treatment herein. Accordingly, exemplary conditions described hereinabove in the context of “protein pre-treatment” shall be taken to apply mutatis mutandis to each and every example describing examples of the method in which the plant tissue is contacted with lysozyme prior to being contacted with the one or more LDH nanoparticles i.e., “lysozyme pre-treatment”. As stated above, exemplary LDH nanoparticles are described herein in the context of the compositions of the disclosure and shall be taken to apply mutatis mutandis to each and every example describing methods of the disclosure. However, in particular examples, the one or more LDH nanoparticles which are contacted with the plant tissue have an average diameter in the range of about 30 nm to about 50 nm. For example, the one or more LDH nanoparticles may have an average diameter of about 30 nm. For example, the one or more LDH nanoparticles may have an average diameter of about 50 nm.

As used herein, the term “plant” when used as a known refers to whole plants, whilst the term “plant tissue”, “plant part” or “part thereof” (in the context of a plant) refers to a plant cell and progeny of same, a plurality of plant cells, a structure that is present at any stage of a plant's development, or a tissue of a plant. Such structures include, but are not limited to, leaves, stems, cutting and scion, flowers, fruits, nuts, roots, seed, seed coat, embryos. The term “plant tissue” includes differentiated and undifferentiated tissues of plants including those present in leaves, stems, flowers, fruits, nuts, roots, seed, for example, embryonic tissue, endosperm, dermal tissue (e.g., epidermis, periderm), vascular tissue (e.g., xylem, phloem), or ground tissue (comprising parenchyma, collenchyma, and/or sclerenchyma cells), as well as cells in culture (e.g., single cells, protoplasts, callus, embryos, etc.). Plant tissue may be in planta, in organ culture, tissue culture, or cell culture.

The plant may be an embryophyte, particularly a sperm atophyte, more particularly an angiosperm [such as a monocotyledon (or monocot), dicotyledon or eudi cotyledon (eudicot)] or a gymnosperm.

Exemplary monocots include plants of the order: asparagales [including amaryllidaccae (such a leek, onion, garlic, shallots and chives) and asparagaceae (such as asparagus)]; areeales [including arecaceae (such as palms, for example coconut palm)]; dioseoreale [including dioscoreaceae (such as yam)]; poales (including bromeliaceae (such as pineapple), Sorghum bicolor (great millet) and poaceae [including corn (maize), wheat, rice, barley, millet, sorghum, oats and bamboo)]; and zingiberales (including musaceae (including banana) and zingiberaceae (including ginger and galangal)).

Exemplary eudicots include plants of the order: Apiales [including apiaceae (such as parsnip, carrot and celery)]; Asterales [including asteraceae (such as lettuce, artichoke and sunflower)]; Brassicales [such as Arabidopsis thaliana (thale cress), and including brassicaceae (such as broccoli, cabbage, kale, cauliflower, brussel sprouts, bak choy, choi sum, kohlrabi, radish, turnip and rapeseed) and capparaeeae (such as capers)]; Caryophyllales [including amaranthaceae (such as spinach, chard and beet) and polygonaceae (such as rhubarb)]; Cucurbitales [including cucurbitaceae (such as cucumber, squash, pumpkin, rockmelon, honeydew melon, zucchini and watermelon)]; Ericales [including aetinidiaceae (such as kiwifruit) and ericaceae (such as blueberry)]; Fabales [including fabaccae (such as various beans, pea, soy bean, mung bean, lentil, peanut and alfalfa)]; Lamiales [including oleaceae (such as olive)]; Malpighiales [including linaceae (such as flax)]; Malvales [including malvaceae (such as cotton)]; Myrtales [including myrtaceae (such as guava)]; Resales [including cannabaceae (such as hemp), rosaceae (such as strawberry, apple, pear, apricot, plum, cherry, peach, raspberry, almond, and nectarine) and moraceae (such as fig)]; Sapindales [including rutaceae (such as citrus, for example orange, lemon, grapefruit, lime and mandarin) and sapindaceae (such, as lychee)]; Solanales [including Nicotiana benthamania (tobacco plants) and Solanum lycopersicum (tomato)] and solanaceae (such as potato, tomato, eggplant, peppers (such as capsicum) and tobacco)]; and Vitales [including vitaceae (such as grape)].

In one example, the plant is selected from the group consisting of a fruiting plant, a leguminous plant, an oil plant, a vegetable plant, a cereal plant, a fibre plant, an ornamental plant, a forestry plant, an aquatic plant, a medicinal plant and a noxious plant or weed. Exemplary crops include cereals, vegetables (including roots and tubers), fruits, pulses, oil crops and fibre crops. Cereals may include corn (maize), rice, wheat, barley, sorghum, millet and oats. Vegetables ma include broccoli, cauliflower, cabbage, artichokes, capers, kale, spinach, lettuce, bok choy, chard, choy sum, leeks, brussel sprouts, kohlrabi, galangal, ginger, celery, rhubarb, asparagus, bamboo shoots, potatoes, sweet potatoes, yams, soybeans, lining beans, alfalfa, carrots, parsnips, beets, radishes, turnips, onions, shallots and garlic. Fruits may include tomatoes, grapes, kiwifruit, berrys (including strawberries, blueberries and rasberries), guava, pears, melons (including rockmelons, watermelons and honeydew melons), citrus (including oranges, mandarins, lemons, limes and grapefruits), stonefruit (including apricots, nectarines, plums, cherries and peaches), lychees, pineapples, figs, apples, bananas, cucumbers, squash, zucchinis, pumpkins, peppers, eggplants and avocados. Pulses may include beans, peas and lentils. Oil crops may include crops from which oil may be obtained, such as palms, soybeans, rapeseeds, sunflower seeds, peanuts, cottonseeds, palm kernels, coconuts and olives. Fibre crops may include cotton, flax, hemp and bamboo.

In another example, the plant tissue can include, for example, cells of seeds, mature and immature embryos, meristematic tissues, seedlings, callus tissue, leaves, flowers, roots, plant buds, gametophytes, sporophytes, pollen and microspores, protoplasts, macroalgae and microalgae. In one examples, the plant tissue may be root tissue or pollen.

In each of the foregoing examples describing methods of introducing one or more nucleic acid molecules to a plant, it will be appreciated that the LDH nanoparticles, and by extension the nucleic acid molecules adsorbed thereto, are internalized into the plant tissue. Preferably, the nucleic acid molecules are taken up via the membrane trafficking pathway of the plant.

As used herein, the term “membrane trafficking” encompasses a wide variety of processes that go into the movement of cargo (typically proteins, pathogens and other macromolecules) using membrane bound transport vesicles. This transport can take place within different organelles in the same cell, or across the cell membrane to and from the extracellular environment. Typically, membrane trafficking can be divided into two basic pathways based on the direction of travel, exocytosis and endocytosis. Exocytosis refers to the movement of cargo out of the cell. Conversely, endocytosis is the movement of cargo into the cell. In one example, the plant uptake of one or more nucleic acid molecules adsorbed onto LDH nanoparticles is via endocytosis.

In each of the foregoing examples describing methods of introducing one or more nucleic acid molecules to a plant tissue, the internalization of the nucleic acid molecules by the plant tissue is increased relative to a method in which the plant tissue is contacted with the LDH nanoparticles in the absence of the protein. Alternatively, or in addition, aggregation of the LDH nanoparticles is decreased relative to a corresponding method in which the plant tissue is contacted with the LDH nanoparticles in the absence of the protein. As used herein, the term “aggregated” or “aggregation” refers to a strong association of primary particles often bound together by, for example, residual chemical treatment, covalent chemical bonds, or ionic chemical bonds. In one example, the aggregation of the LDH nanoparticles is reduced when the LDH nanoparticles are coated in a protein. In another example, the aggregation of the LDH nanoparticles is reduced when the LDH nanoparticles are coated in lysozyme.

In some examples, the method of the disclosure is performed to introduce to a plant tissue one or more nucleic acid molecules capable of silencing expression of a gene in the plant, or somatic or heritable editing the plant's genome, or modifying (e.g., increasing or decreasing) expression of a gene in the plant, or directing expression of an exogenous gene in the plant. Accordingly, it follows that the one or more nucleic acid molecules are capable of gene silencing, or somatic or heritable gene editing, modifying expression of a gene, or expressing an exogenous gene, in the plant to which they are introduced.

As used herein, the term “gene silencing” refers to a phenomenon whereby a function of a gene is completely or partially inhibited. Exemplary nucleic acids which are capable of gene silencing are RNA interference (RNAi) molecules. Interfering RNAs affect sequence-specific, post-transcriptional gene silencing in animals and plants by base pairing to the mRNA sequence of a target nucleic acid. Techniques for the design of such molecules for use in targeted inhibition of gene expression are well known to one of skill in the art. Exemplary RNAi molecules include, but are not limited to, double stranded RNA (dsRNA), small interfering RNA (siRNA), single stranded RNA (ssRNA), short hairpin RNA (shRNA) and short hairpin microRNA (shmiR) molecules. Accordingly, in some examples, the one or more nucleic acid molecules may be RNAi molecules targeting mRNA transcripts associated with a gene of interest or nucleic acids encoding said RNAi molecules. For example, the gene of interest may be associated with a plant pathogen or pest. For example, the gene of interest may be associated with an agronomic trait (such as production of phenolic compounds, uniformity in flowering and maturity, days to anthesis, germination rate, grain weight or size (e.g., thousand seed weight), awn length, spike length, plant density, plant height, total biomass, oil content, fertilizer utilization and responsiveness, water usage, growth rate, for example).

As used herein, the term “gene editing” and the like refers a type of genetic engineering in which a nucleotide is inserted, replaced, or removed from a genome using gene editing tools. Examples of gene editing tools include, without limitation, zinc finger nucleases, TALEN and CRISPR. Gene editing may be somatic genome editing i.e., by introducing changes that are not heritable, or germline gene editing i.e., by introducing changes that are heritable.

As used herein, the term “modifying expression”, particularly of a gene, refers to modifying the transcription of an encoding DNA sequence into mRNA, or alternatively modifying the translation of said mRNA into a peptide, polypeptide, or protein, relative to a reference level of expression. Accordingly, it follows that “increasing expression” refers to increasing the transcription of an encoding DNA sequence into mRNA, or alternatively increasing the translation of said mRNA into a peptide, polypeptide, or protein, relative to a reference level of expression. Similarly, “decreasing expression” refers to decreasing the transcription of an encoding DNA sequence into mRNA, or alternatively decreasing the translation of said mRNA into a peptide, polypeptide, or protein, relative to a reference level of expression. In some examples, the method comprises introducing one or more nucleic acid molecules that increases expression of a gene of interest in the plant or plant tissue. In other examples, the method of the disclosure comprises introducing one or more nucleic acid molecules that decreases expression of a gene of interest in the plant or plant tissue. The gene of interest may be associated with a plant pathogen or pest. Alternatively, or in addition, the gene of interest may be associated with an agronomic trait (such as production of phenolic compounds, uniformity in flowering and maturity, days to anthesis, germination rate, grain weight or size (e.g., thousand seed weight), awn length, spike length, plant density, plant height, total biomass, oil content, fertilizer utilization and responsiveness, water usage, growth rate). Alternatively, or in addition, the gene of interest may be associated with an ornamental trait.

In some examples, the one or more nucleic acid molecules which are introduced to the plant or plant tissue comprise a polynucleotide sequence encoding a protein or polypeptide of interest or a functional fragment thereof. In accordance with this example, the one or more nucleic acid molecules may be messenger RNA (mRNA), PCR-amplified DNA, or plasmid DNA (pDNA) molecules comprising a polynucleotide sequence encoding the protein or polypeptide of interest or functional fragment thereof. In some examples, the protein or polypeptide of interest or functional fragment thereof which is encoded by the one or more nucleic acids may be endogenous to the plant, and may be expressed in either a wildtype or modified form. In other examples, the one or more nucleic acid molecules which are introduced to the plant or plant tissue comprise a transgene of interest. In either case, the polynucleotide encoding the endogenous protein (modified or otherwise) and/or the transgene of interest may confer a phenotype or trait of interest in the plant. In one example, the phenotype or trait of interest may be protection or resistance to a plant pathogen or pest. In another example, the polynucleotide encoding the endogenous protein (modified or otherwise) and/or the transgene may be associated with and/or improve an agronomic trait (such as production of phenolic compounds, uniformity in flowering and maturity, days to anthesis, germination rate, grain weight or size (e.g., thousand seed weight), awn length, spike length, plant density, plant height, total biomass, oil content, fertilizer utilization and responsiveness, water usage, growth rate, for example). Of course, the skilled person will appreciate that any nucleic acid may be introduced via the method or nanoparticles of the disclosure. The polynucleotide or transgene of interest may be operably linked to one or more regulatory elements, such a promoters or enhancers. The polynucleotide or transgene may further comprise or be associated with a suppressor of post-transcriptional and transcription gene silencing (e.g. the p19 suppressor of gene silencing) to ensure that the polynucleotides or transgenes of interest delivered on the nanoparticles are not switched off by gene silencing. The polynucleotide or transgene of interest may also comprise one or more introns to improve/enhance expression of the polynucleotide or transgene. In some examples, the polynucleotide or transgene sequence encoding a protein of interest, or functional fragment thereof, may comprise a mobile transfer RNA (tRNA) in the 3′ UTR to facilitate systemic expression and translocation of the expression product throughout the plant.

In some examples, the introduction of the nucleic acid molecule to the plant protects the plant from a plant pest and/or improves resistance of the plant to an biotic stress and/or an abiotic stress and/or improves one or more agronomic traits of interest and/or improves quality and value of harvest value. For example, a target plant of interest can be modified according to the method described herein through targeted gene modification to protect against biotic or abiotic stress. For example, a nucleic acid may be introduced to a plant or plant tissue using the method of the disclosure, wherein the nucleic acid molecule silences a gene associated with a plant pest or pathogen. For example, a nucleic acid may be introduced to a plant or plant tissue using the method of the disclosure, wherein the nucleic acid molecule increases expression of a gene which confers resistance of a plant to a plant pathogen or plant pest. For example, a nucleic acid may be introduced to a plant or plant tissue using the method of the disclosure, wherein the nucleic acid molecule increases expression of a gene which confers resistance of a plant to an abiotic stress. For example, a nucleic acid may be introduced to a plant or plant tissue using the method of the disclosure, wherein the nucleic acid molecule increases expression of a gene which improves one or more agronomic traits of interest. For example, a nucleic acid may be introduced to a plant or plant tissue using the method of the disclosure, wherein the nucleic acid molecule increases expression of a gene which improves quality and/or value of a harvested plant part.

As used herein, the term “biotic stress” refers to those living substances which cause one or more injuries to a plant. Examples of biotic stresses include those injuries resulting from infections by insects, nematodes, snails, mites, weeds, pathogens, such as fungus, bacteria or viruses, and physical damage caused by people and animals (i.e. grazing, tredding, etc.). In some examples, the one or more nucleic acid molecule protects the plant from a biotic stress which is a plant pest that is an insect pest, a viral pathogen, a fungal pathogen, a nematode, a bacterial pathogen or a parasite. As used herein, the term “abiotic stress” refers to those non-living substances or environmental factors which can cause one or more injuries to a plant. Examples of abiotic stresses include those injuries which result from chilling, freezing, hail, flooding, drought, soil compaction, soil crusting and agricultural chemicals such as pesticides and herbicides.

In accordance with examples in which the introduction of the one or more nucleic acid molecules protects the plant from a biotic or abiotic stress, the plant may be protected for at least 15 days, or at least 20 days, or at least 25 days, or at least 30 days weeks, from the time the one or more nucleic acid molecules are introduced to the plant tissue. In other examples the plant may be protected for 2 to 8 weeks, or 3 to 6 weeks, or 4 to 5 weeks, from the time the one or more nucleic acid molecules are introduced to the plant tissue. In this regard, once administered to a plant, the LDH nanoparticles, or composition comprising same, may degrade at a rate of 10-30% per week (e.g., at a rate of 15-25% per week, or at a rate of 15-20% per week). The duration of protection may be affected, for example, by the amount of rainfall on the plant. Therefore, in a drier climate it is expected that the duration of protection would be increased, whereas a shorter duration of protection may be provided in a wetter climate. Depending on the duration of protection anticipated, the method of the disclosure may be performed on a periodic basis to maintain protection of the plant. For example, the method of the disclosure may be performed weekly, or fortnightly, or monthly, or quarterly. In other examples, the method of the disclosure may be performed once or periodically at a particular time of year when the abiotic or biotic stress if prevalent. In yet other examples, the method of the disclosure may be performed on a one off basis.

### EXAMPLES

### Example 1. Lysozyme-Coated Ldh-30 Nanoparticles as Universal Nanocarriers for Delivery Of Functional Nucleic Acids into Plants

The inventors demonstrated that lysozyme-coated layered double hydroxide (LDH) nanoparticles are able to access the membrane trafficking pathway of plants to efficiently deliver various forms of functional nucleic acids into plant roots, including i) mRNA into N. benthamiana, Arabidopsis thaliana, tomato, and sorghum roots, where it is subsequently translated into protein; ii) plasmid DNA into N. benthamiana and sorghum roots, where it is efficiently expressed into protein; and iii) small interfering RNA and long double-stranded RNA into intact N. benthamiana roots to induce RNA interference of a target gene. The results showed that lysozyme-coated LDH nanoparticles are not only internalized by plant root cells but also translocated to the vascular cylinder and above-ground tissues.

### Materials and Methods

### Nanoparticle Synthesis and Protein Encapsulation

The LDH nanoparticles with the average diameter of 30-40 nm were synthesized according to the previous report with slight modifications (Yong, J., et al., Sheet-like clay nanoparticles deliver RNA into developing pollen to efficiently silence a target gene. Plant Physiol, 2021. 187 (2): p. 886-899). Briefly, Mg(NO₃)₂ and Al(NO₃)₃ methanol solution were poured into NaOH methanol solution under rigorous stirring for 20 min. The obtained slurry was washed and re-dispersed in de-ionized water to obtain the homogeneous suspension with the average particle size of 30-40 nm. The LDH nanoparticles with average sizes of 50 nm, 80 nm and 120 nm were prepared by hydrothermal treating 30 nm LDH in Teflon lined autoclave at 100 °C for 1 h and 10 h, and at 200 °C for 10 h, respectively. Hydrodynamic size and zeta potential was analysed using Zetasizer. The size and thickness of the nanoparticles was also confirmed by measuring and averaging the lateral diameter of 50-100 particles in AFM and TEM images using ImageJ. Fluorescein (Sigma-Aldrich F2456) was introduced to LDH nanoparticles through intercalation by stirring fluorescein (5% total mass of LDHs) with LDHs for 2 h, followed by washing and redispersing in de-ionized water. Lysozyme or BSA (bovine serum albumin) were coated onto nanoparticles at a mass ratio of 5:1 (protein:LDH) through electrostatic interactions by dropwise adding LDH suspension into protein solution and stirring for 30 min without washing. The coating process was performed after fluorescein was intercalated into the nanoparticle layers or biomolecules were loaded to the surface of the LDH nanoparticles. The lysozyme was from chicken egg white (Sigma-Aldrich 62970) and the BSA used was BSA heat shock fraction (Sigma-Aldrich A7906).

### Plant Culture and Treatment

Seeds of Nicotiana benthamiana (non-transgenic, RFP:ER and 16c) and Arabidopsis thaliana (ecotype Col-0) were surface sterilized and germinated vertically in petri dish with Murashige and Skoog solid media in growth room (20-22 °C day time and 16-18 °C night time, 12 h/12 h). Tomato (Solanum lycopersicum, variety Moneymaker) seeds were surface sterilised and germinated in water for 5 days and grown in potting mix in growth room for 4 weeks and transferred to glass house with artificial light (16 h/8 h day/night). Sorghum bicolor seeds were surface sterilized and germinated in water for 9 days and collected for root treatment. Sorghum callus cultures were induced from immature embryos in 10 cm petri plates as described previously in Liu et al., (2015) Moench. South African Journal of Botany 98:157-160. All experiments with intact plant seedlings, isolated shoots and sorgum callus were performed in the laboratory and growth room until harvest for analysis. Injection of flower buds was conducted on intact tomato plants in the glasshouse.

Loading of Biomolecules onto LDH Nanoparticles

Biomolecules, including mRNA, dsRNA, siRNA and pDNA were loaded on as-prepared LDHs by slowly dripping LDH suspension into biomolecule suspension, followed by occasional vortex and aging at room temperature for 2 h. The optimal LDH to nucleic acid mass ratio for saturation loading of the nanoparticles was determined by electrophoresis of the loading mixture and ranged from 3:1 to 7:1 depending on the type of nucleic acid loaded. When molecules are completely loaded onto nanoparticles, their bands should not migrate but stay in the loading well.

After the mRNA, dsRNA, siRNA or pDNA was loaded onto nanoparticles, the complex was coated with lysozyme and added into hydroponic culture media of 3-4-week-old non-transgenic N. benthamiana seedlings at an RNA or DNA concentration (as appropriate) of 10 mg/L.

Seedling, Leaf and Callus Treatment with Nanoparticles

Seedlings were germinated on 1× Murashige and Skoog plates and, at the desired age, were transferred to a 12-well-plate with 1 ml of incubation media (20 mM 2-(N-morpholino) ethanesulfonic acid buffer, pH 6.0) and containing the nanoparticles loaded with fluorescein or nucleic acid. The roots were immersed in incubation media while any shoots outside the liquid were left hanging in the wells without touching the liquid. The treated seedlings were placed in the growth room for a certain time until harvest.

For inhibited internalization experiments the seedlings were exposed to 100 mM Brefeldin-A or 50 mM Wortmannin for 30 min before adding LDH nanoparticles into culture media. In cold treatment, the seedlings were moved to the cold room 30 min prior to adding LDH nanoparticles, and then incubation was continued for 4 h in the cold room.

Mature leaves of N. benthamiana plants were infiltrated with 50 μl of MES buffer (pH 6.0) containing the nanoparticles loaded with plasmid DNA. Sorghum callus was removed from petri plates and immersed into falcon tube containing 5 ml MES buffer (pH 6.0) containing the lysozyme-coated nanoparticles loaded with GFP mRNA.

For whole seedling experiments involving inhibitors, the roots were exposed to 100 mM Brefeldin-A or 50 mM Wortmannin for 30 min before adding the nanoparticles into culture media. For cold treatment, the seedlings were moved to the cold room 30 min prior to adding the nanoparticles, and then incubation was continued for 4 h in the cold room.

For the lysozyme pre-treatment experiment, roots were treated in 20 mM MES (pH 6.0) with 1 mg/ml lysozyme for 4 h before nanoparticle internalization experiment. Roots were rinsed three times by dipping into fresh 20 mM MES (pH 6.0) before transferring to 20 mM MES (pH 6.0) with 200 mg/l fluorescein-labelled LDHs for incubation of 2 h.

To assess the effect of nanoparticle treatment on expression of genes encoding components of the membrane trafficking pathway, Arabidopsis roots were treated for 2 days with or without nanoparticles prior to RNA extraction and qRT-PCR analysis of EPSINI, VPS60.1 and VPS36 mRNA levels relative to ACTIN mRNA. The RNA was extracted with TRIzol (Thermo Fisher Scientific) following the manual and reversed transcribed using first strand cDNA synthesis kit (Thermo Fisher Scientific) following the manual. RNA extracted from 10 roots together was annotated as one biological replicate. ΔΔCT method against Actin mRNA as housekeeping gene and blank control group was applied for data processing of the results.

### Uptake of Nanoparticles by Developing Pollen

To assess the effect of lysozyme pre-treatment on the in vitro uptake of nanoparticles by tomato pollen, early bicellular pollen was extracted from 5-7 mm tomato flower buds by gently breaking the anther with scalpel blade in 10% glycerol as previously described in Yong et al. (2021) Plant Physiol 187:886-899. Pollen was the treated in 20 mM MES (pH6.0) with or without 1 mg/mL lysozyme for 4 h before the addition of nanoparticles. Pollen of all groups was washed three times before transferring to 20 mM MES (pH6.0) with 50 mg/L fluorescein labelled LDH30 or lysozyme coated LDH50. After 2 hrs of incubation with nanoparticles, the pollen was then washed three times and re-suspended in 10% glycerol and subjected to flow cytometer (CytoFLEX) for quantitative analysis. Fluorescence intensities in fluorescein channel of 10000 pollen were obtained and averaged as one replicate in certain group.

In Planta Delivery of a Functional Transgene into Developing Tomato Pollen

For flower bud injection, an insulin syringe was used to inject 10-20 μl of the nanoparticle solution into the anther region of 4-6 mm flower buds of tomato. The nanoparticle solution contained lysozyme-coated LDH30 loaded with plasmid DNA encoding a 35S:GFP transgene (pDNA-1, DNA concentration 20 mg/L) in 20 mM MES (pH 6.0). The injected flower buds remain attached to the tomato plants for 3 days post injection and then the pollen was extracted and assessed for GFP expression using a flow cytometer (CytoFLEX) and confocal microscopes.

### Fluorescence Imaging for Quantitative Analysis

Confocal microscopy images were obtained using a Leica SP8 Laser Scanning Confocal Microscopy and a Zeiss LSM900 Laser Scanning Confocal Microscope. The excitation and emission wavelength used for detection of fluorescein, RFP, GFP and YFP, as well as other microscope settings are shown in Table 1.

TABLE 1
Confocal microscope settings

| | | λ_{Excitation} | λ_{Emission} | Laser | Detector |
|---|---|---|---|---|---|
| Experiment | Fluorophore | (nm) | (nm) | intensity | and gain |
| RFP | Fluorescein | 490 | 500-580 | 100% | PMT 700 |
| colocalization | RFP | 590 | 600-700 | 100% | PMT 800 |
| FM4-64 | Fluorescein | 490 | 500-580 | 100% | HyD 200 |
| colocalization | FM4-64 | 490 | 650-789 | 100% | PMT 1000 |
| Other LDH | Fluorescein | 490 | 500-600 | 100% | PMT 700 |
| uptake | Chlorophyll | 490 | 650-750 | 100% | PMT 700 |
| mRNA | GFP | 483 | 493-600 | 50% | HyD 200 |
| delivery | | | | | |
| RNAi | GFP | 480 | 490-600 | 10% | HyD 80 |
| UBI:GFP | GFP | 471 | 481-600 | 100% | HyD 200 |
| delivery | | | | | |
| 35S:GFP | GFP | 483 | 493-600 | 100% | PMT 800 |
| delivery | | | | | |

For quantitative analysis, one primary root from an individual seedling was regarded as one biological replicate, and 4-5 non-overlapping fields of view with approximately 5 mm distance from one another were captured, measured, and averaged to obtain the value of one biological replicate. The images were processed with Leica LasX application suite and Zen Blue. The average intensity and Pearson's colocalization coefficient were analysed through ImageJ. The fluorescence intensity was normalized based on blank or no treatment group of mature root sections.

The whole seedling/root photographs and sorghum callus images were obtained using a Cannon EOS 600D excited by 4 Dark Reader Hand Lamps (Clare Chemical Research), and an orange filter was set to exclude the interference of blue excitation light. The aperture was set at F7.1 and exposure time 5 s.

### Statistics Analysis

Data are represented as mean #standard error of mean (SEM), n=3. Biological replicates stand for individually treated seedlings, callus, and flowers, where the exact value of each replicate was indicated by diamond in the column graphs. Statistical significance between more than two groups was determined through one-way ANOVA followed by post hoc Tukey's multiple comparisons for significance between groups, or by a two-tailed t-test for two groups. Root tip and mature sections were analysed separately.

### Results

### Protein-Coated LDH Nanoparticles

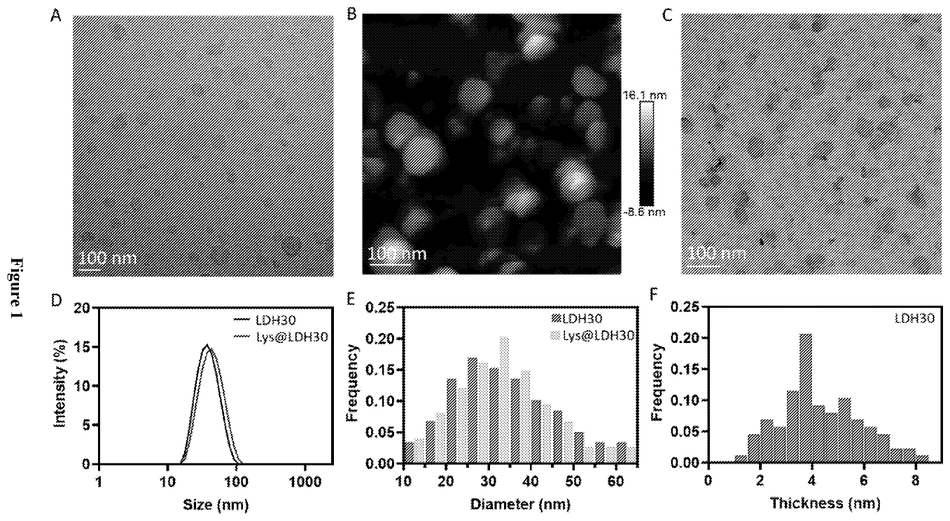

LDH nanoparticles were prepared and characterized using transmission electron microscopy (TEM) images, atomic force microscopy (AFM) images and dynamic light scattering analysis (DLS). TEM and AFM images confirmed hexagonal plate-like morphology of LDH nanoparticles with an average lateral dimension of ~35.2 nm (FIGS. 1A and B). The dynamic light scattering analysis (DLS) data similarly estimated the hydrodynamic particle size of LDH nanoparticles to be in the range 15-100 nm, with an average of ~37.3 nm (FIGS. 1D and E, Table 2). Atomic force microscopy (AFM) also confirmed the plate-like nanostructure and an average lateral size of ~39.3 nm (FIG. 1B), which is consistent with TEM and DLS data (Table 2). Given their average lateral size of 30-40 nm, the inventors subsequently referred to the nanoparticles as “LDH30”. The thickness of the LDH30 nanoparticles was determined by AFM to range from ~2 to 7 nm, with an average thickness of ~4.4 nm (FIGS. 1B and F. These data indicate that the sheet-like LDH30 nanoparticles have a diameter/thickness ratio of 8 to 9. For comparison, LDH50, LDH80 and LDH120 nanoparticles were also prepared (Table 2).

TABLE 2
Characterization properties of LDHs.

| | zeta-average | zeta- | |
|---|---|---|---|
| | size | potential | |
| Sample | (nm) | (mV) | PDI |
| LDH30, 20 mM KCl | 37.3 | 45.5 | 0.207 |
| LDH30, 20 mM KCl | 50.5 | 30.7 | 0.259 |
| Lys@LDH30 | 38.4 | 18.7 | 0.252 |
| Lys@LDH30, 20 | 54.9 | 23.1 | 0.314 |
| mM KCl | | | |
| HT-Lys@LDH30, 0 | 39.2 | 22.8 | 0.248 |
| mM ^{a} | | | |
| Lysozyme ^{b} | ~4 ¹ | 8 | |
| BSA@LDH30 | 41.2 | −15.5 | 0.341 |
| BSA | ~10 ² | −18 | |
| Congo red-LDH | 61.2 | 33.2 | 0.354 |
| LDH50 | 50.7 | 42.6 | 0.141 |
| LDH80 | 87.8 | 40.9 | 0.212 |
| LDH120 | 126.2 | 47.3 | 0.246 |

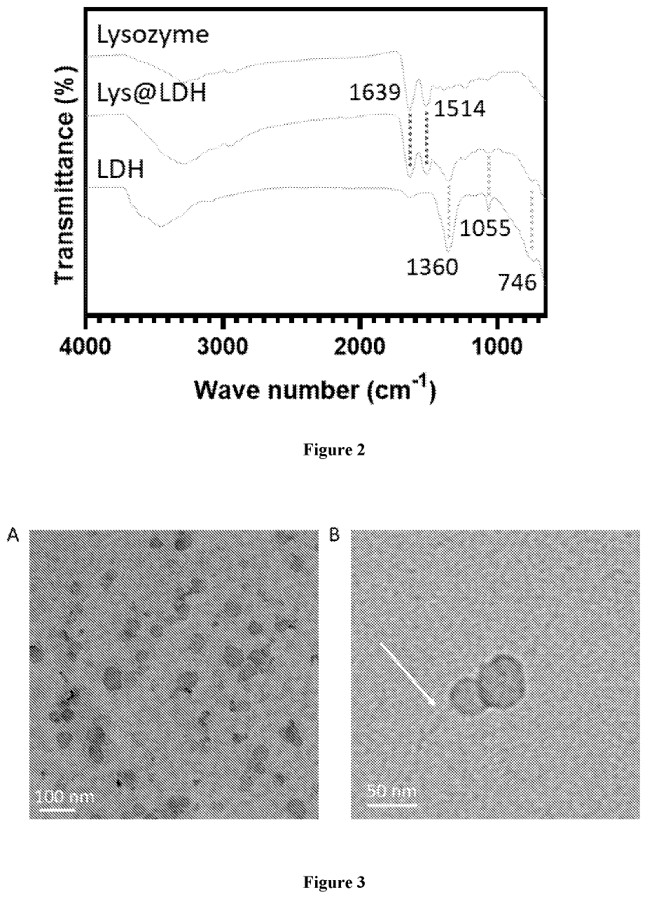

The LDH30 nanoparticles were subsequently coated with either lysozyme or bovine serum albumin (BSA), and Fourier-transform infrared spectroscopy (FTIR) was used to confirm the protein encapsulation. For example, the major absorption peaks of lysozyme (amide I: 1639 cm⁻¹; amide II: 1514 cm⁻¹) and the LDH30 nanoparticles (1360, 1055 and 746 cm⁻¹ of vibrations of intercalated nitrate/carbonate anions and Metal-O bonds) were all observed in the spectrum of the lysozyme-coated nanoparticles (FIG. 2). The protein coating did not affect their overall sheet-like morphology but increased the average particle size by ~1-4 nm (Figure. 1C-E; Table 2). However, the lysozyme and BSA coatings decreased the surface charge density of the nanoparticles from 45.5 mV to 18.7 mV and −15.5 mV, respectively (Table 2).

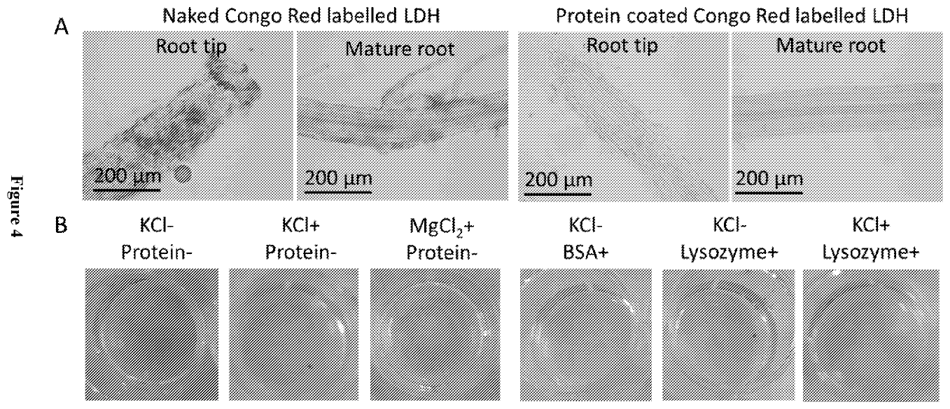

Plant roots can take up whole protein and the colloidal stability of LDH30 nanoparticles is greatly improved by coating protein. The LDH30 nanoparticles were therefore subsequently coated with either lysozyme or bovine serum albumin (BSA), by dropwise addition of LDH nanoparticle suspension to lysozyme or BSA solution respectively under vigorous stirring conditions to achieve a final protein/LDH mass ratio of 5:1. The protein coating/encapsulation was confirmed by Fourier-transform infrared spectroscopy (FTIR) (FIG. 2). The characteristic absorption peaks of lysozyme (e.g., at 1639 cm⁻¹ of amide I band and 1514 cm⁻¹ of amide II band), and LDH30 nanoparticles (e.g., at 1360, 1055 and 746 cm⁻¹ of vibrations of intercalated nitrate anions and Metal-O bonds) were both observed in the FTIR spectrum of lysozyme-coated LDH30 nanoparticles, hereafter referred to as Lys@LDH30 nanoparticles (FIG. 2). The protein coating did not affect the overall sheet-like morphology of LDH30 nanoparticles (FIGS. 3A and B), but slightly increased the average particle size from 37.3 to 38.4 nm (FIG. 1D, Table 2), and resulted in a slightly larger the particle size distribution. The lysozyme and BSA coatings also decreased the surface charge of the LDH nanoparticles from 45.5 (LDH30) to 18.7 mV and −15.5 mV, respectively (Table 2).

### Uptake of Nanoparticles by Roots

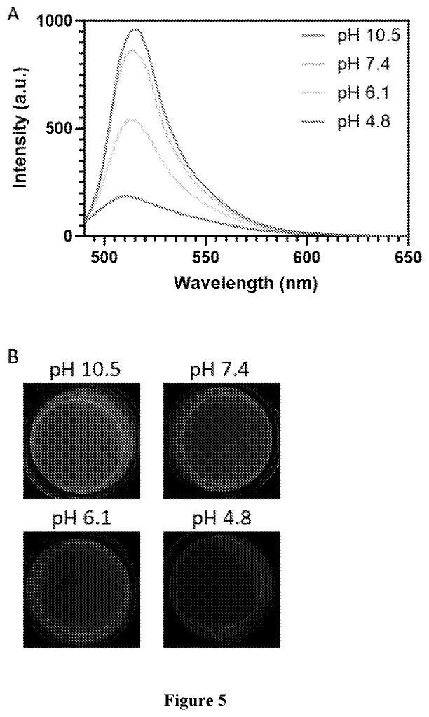

Positively-charged LDH nanoparticles without a lysozyme or BSA coating and labelled with Congo Red for visualization (Table 2) formed aggregates on the root surface after 24 hr, especially around the root tip (FIG. 4A) and in hydroponic culture media (FIG. 4B). The inventors hypothesized that aggregation of the nanoparticles on the root surface and in culture media was due to the positive charge of the LDH nanoparticles and binding of the nanoparticles to negatively charged solutes (e.g., organic acids) and root mucilage typically found in root exudates (FIG. 4). The inventors solved the problem of aggregation by increasing the concentration of KCl in the culture media to 20 mM (FIG. 4B). In contrast, the addition of MgCl₂ did not reduce aggregation, suggesting that the potassium ion minimized the formation of the aggregates (FIG. 4B) by inhibiting the efflux of negatively charged solutes (e.g., such as organic acid anions) in the root exudate. In addition to decreasing the positive surface charge of the nanoparticles, the protein coating of the LDH nanoparticles decreased the magnitude of the positive surface charge density, increased colloidal stability and reduced the formation of aggregates on the root surface and in culture media, even in the absence of 20 mM KCl (FIG. 4B). As a result, an addition of 20 mM KCl to the hydroponic growth media was favoured in subsequent experiments to prevent the aggregation of the LDH30 nanoparticles.

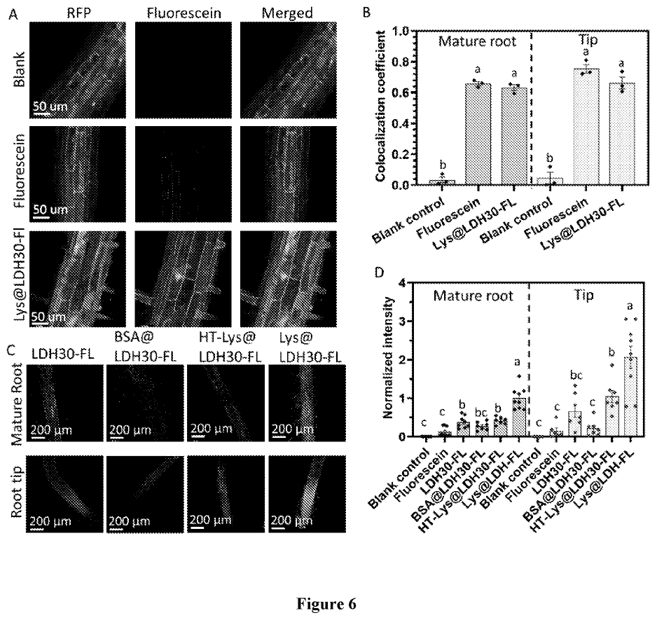

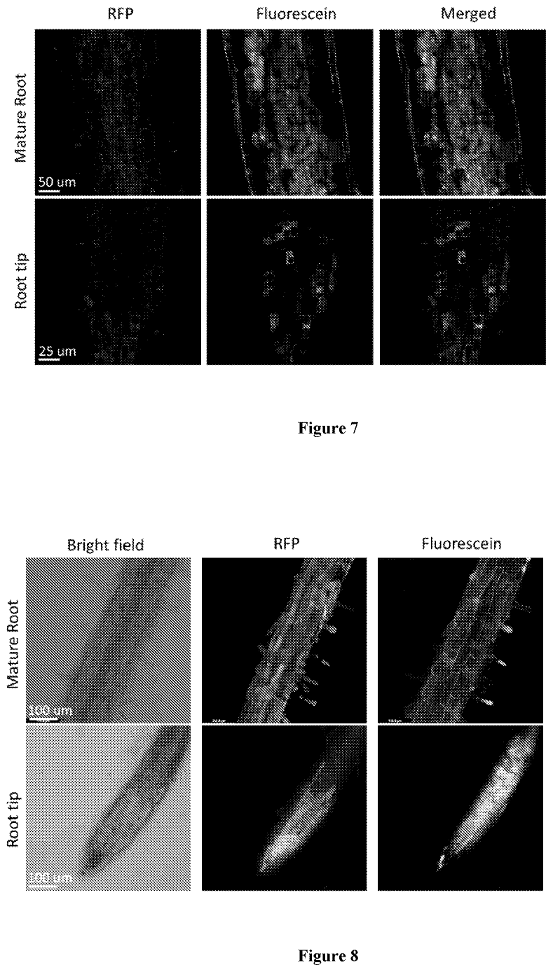

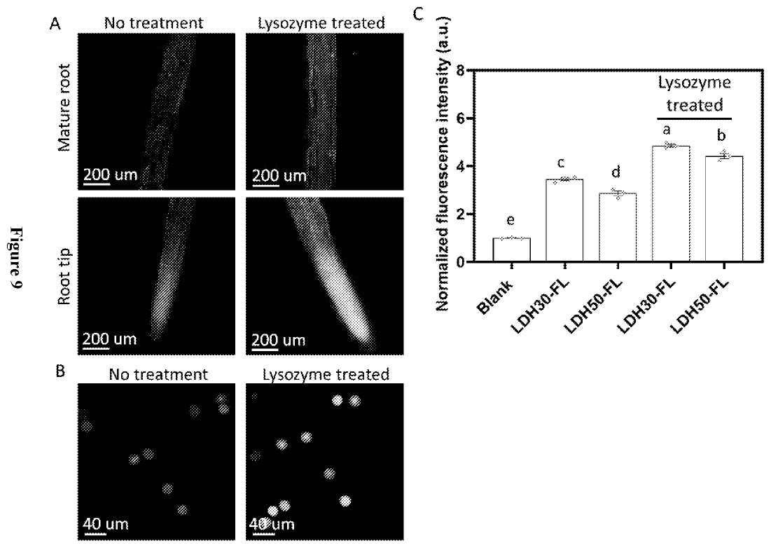

To confirm the uptake and the cellular internalization of lysozyme-coated LDH nanoparticles, the particles were labelled with pH sensitive dye fluorescein (FIG. 5, Lys@LDH30-FL) and uptake by the roots was tested. Specifically, the inventors used a transgenic line of Nicotiana benthamiana expressing a endoplasmic reticulum (ER)-localized Red Fluorescence Protein reporter (RFP:ER), where the RFP signal attached to ER is adopted as the indicator of cell periphery (cell cytosol and membrane). As illustrated in FIG. 6A, the fluorescein green signal of Lys@LDH30-FL was observed in the root epidermal and cortex layer 4 h after treatment with Lys@LDH30-FL. This strong green fluorescence observed in roots treated with the lysozyme-coated nanoparticles was in sharp contrast to the weak fluorescence signal observed in the untreated control roots and roots treated with free fluorescein, thereby demonstrating the specific and efficient uptake of Lys@LDH30-FL nanoparticles by N. benthamiana roots. The inventors also analysed the cellular internalization of the nanoparticles using the pixel-to-pixel Pearson's co-localization coefficient between the nanoparticle signals and the RFP:ER as an indicator of the intracellular area (FIG. 6B). The co-localization coefficient was ~0.66 in the root tip and ~0.63 in the mature root, which confirmed that a large proportion of the lysozyme-coated nanoparticles were located in the cell periphery. Combining with the high magnification images demonstrating the LDH nanoparticles inside the cells (FIG. 7), these data suggest that the lysozyme-coated nanoparticles were efficiently taken up by roots and were readily internalized by the root cells. A striking observation was the higher amount of uptake and internalization of lysozyme-coated LDH30 nanoparticles in the root cell elongation zone (i.e., root tip within 1 mm from the root cap/end) compared to the mature regions of the root (FIG. 6C and FIG. 8). As a result, the inventors analysed the root tip and mature root separately in subsequent experiments.

The inventors initially hypothesized that all forms of protein coating of the LDH30 nanoparticles would enhance the nanoparticle internalization via endocytosis of the proteinaceous complex. To assess how different protein coatings affect the nanoparticle uptake, the inventors compared the uptake of Lys@LDH30-FL with particles coated with bovine serum albumin (BSA@LDH30-FL) and high temperature treated lysozyme (HT-Lys@LDH30-FL). Surprisingly, not all protein coatings lead to the efficient internalization of the nanoparticles. Indeed, as shown in FIG. 6B, lysozyme-coated LDH30 (Lys@LDH30-FL) nanoparticles increased the fluorescence intensity by at least 2-fold compared to uncoated LDH nanoparticles (FIGS. 6C and D), whereas coating of the LDH30 nanoparticles with BSA (BSA@LDH30-FL) had the opposite effect and reduced internalization by the root. This was in spite of the BSA-coated LDH30 nanoparticles being colloidally stable and not aggregating on the roots or in hydroponic culture media (FIG. 4B), which typically enables mammalian cells to take up LDH nanoparticles coated with BSA. Similarly, the heat-treated lysozyme coating also failed to enhance the nanoparticle uptake significantly compared to naked LDH30-FL. These contrasting uptake/internalisation efficiency of lysozyme-, heat-treated lysozyme-, and BSA-coated LDH nanoparticles (FIGS. 6C and D) may be related to the surface charge of protein-coated nanoparticles and the hydrolase activity of lysozyme. In this regard, the BSA@LDH30-FL nanoparticles are negatively charged (−15.5 mV), whereas the uncoated nanoparticles (LDH30-FL), lysozyme-coated nanoparticles (Lys@LDH30-FL) and heat-treated lysozyme-coated nanoparticles (HT-Lys@LDH30-FL) possessed a positive surface charge (45.5, 18.7 mV and 22.8 mV, respectively, Table 2). These results indicate that a positive surface charge may enhance the affinity of the nanoparticles for the negatively charged cell surface and/or cytoplasm immediately inside the cell membrane.

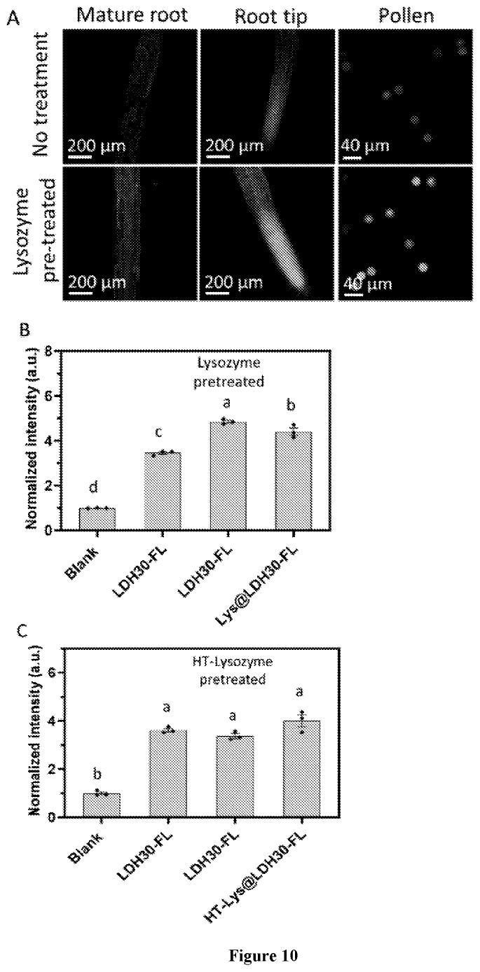

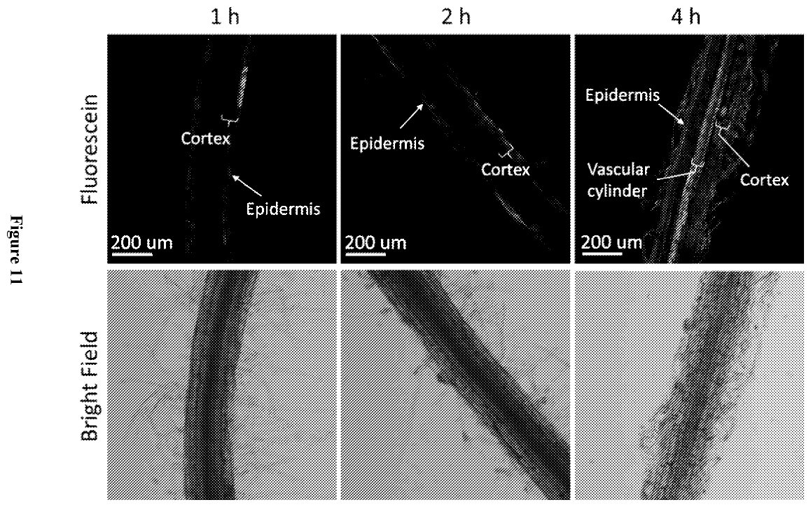

Additionally, and unrelated to the charge of the nanoparticles, the inventors hypothesized that the glycoside hydrolase activity of lysozyme coated on the nanoparticle surface may help degrade cell wall polysaccharides, making it more porous and readily enhancing the movement of the nanoparticles through the cell wall to the cell membrane for internalization. To test this hypothesis, the inventors investigated the impact of pre-treatment of plant tissue with lysozyme or heat-treated lysozyme on the internalization of naked LDH30-FL and LDH50-FL nanoparticles by N. benthamiana root and developing pollen of tomato (Solanum lycopersicum). The N. benthamiana roots and early bicellular pollen were treated with 1.0 mg/mL lysozyme or heat-treated lysozyme for 4 h and then washed with fresh 20 mM MES buffer (pH 6.0) to remove free lysozyme or free heat-treated lysozyme. Controls were not pre-treated. The tissues were then incubated for 2 h in LDH-FL-containing MES buffers prior to confocal microscopy or flow cytometry analysis. As illustrated in FIGS. 9A and B, lysozyme-treated roots and pollen showed a significant increase in fluorescence intensity from internalised LDH-FL compared to the control roots and pollen not pre-treated with lysozyme. Specifically, the pre-treatment of pollen with lysozyme increased internalization of LDH30-FL and LDH50-FL nanoparticles by 40% and 52%, respectively (FIG. 9C). Meanwhile, pre-treatment of tissue with HT-lysozyme did not increase uptake of LDH nanoparticles (FIG. 10C). These data clearly indicate that lysozyme treatment of plant tissue enhances the internalization LDH nanoparticles, and that lysozyme activity assists with this internalisation, probably by degrading polysaccharides in the plant cell wall and increasing its permeability.

In view of the inventors' finding that the uptake of Lys@LDH30-FL by the root tip region and mature root is different, the inventors quantified the uptake amount by these two regions of the root separately for comparison. Quantitative analysis indicated that the fluorescence of Lys@LDH30-FL treated roots was significantly increased compared to that of the free-fluorescein treated and blank roots (FIG. 6D). For example, the intensity of mature roots and root tips in Lys@LDH30-FL treated group is >3 and >8 times that in free fluorescein treated group, respectively, where the normalized intensity of the tip taking up Lys@LDH-FL is higher than that of the mature root. The lysozyme-coating almost doubled the intensity of naked LDH30-FL, consistent the inventors earlier observation. The pixel-to-pixel Pearson's co-localization coefficient of fluorescein signals with intracellular RFP:ER was estimated as an indicator of the cellular internalization of nanoparticles (FIG. 6B). The Lys@LDH30-FL-treated roots had a co-localization coefficient of ~0.63 in the mature root and ~0.66 in the root tip, significantly higher than that in the blank control group, confirming that the LDH30 nanoparticles were internalized by root cells. Interestingly, free fluorescein treatment led to a similar co-localization coefficient with RFP:ER (FIG. 6B) but a much lower fluorescence intensity (FIG. 6D), suggesting that the root cells took up very little free fluorescein and that the fluorescein fluorescence signal was attenuated outside the cell due to the more acidic nature of the plant apoplast. Collectively, these data demonstrate that Lys@LDH30-FL nanoparticles are efficiently taken up by roots and internalized by root cells.

The time-course analysis of nanoparticle uptake (FIG. 11) revealed that Lys@LDH30-FL nanoparticles are first internalized by the epidermal cells on the mature root surface at 1-h post incubation. The internalized nanoparticles are then moving to the cortex region at 1-2 h, and eventually throughout the whole root, including the vascular system at 4 h post incubation (FIG. 11).

### Membrane Trafficking Pathway is Required for Uptake and Translocation of Lysozyme-Coated LDH Nanoparticles

To investigate the mechanism of uptake and translocation of lysozyme-coated nanoparticles by plant tissues, inhibitors of clathrin-mediated endocytosis (Wortmannin) and protein secretion pathway (Brefeldin-A) were applied to root tissues prior to and during treatment with lysozyme-coated nanoparticles. The roots were first pre-treated with the inhibitors for 30 min prior to the addition of Lys@LDH30 nanoparticles for 4 h. The inventors also assessed the effect of cold treatment on Lys@LDH30-FL nanoparticle uptake by the root. In this case, the plants were first pre-incubated in cold room at 4 °C for 30 min prior to the addition of Lys@LDH30 nanoparticles for 4 h culture at 4 °C.

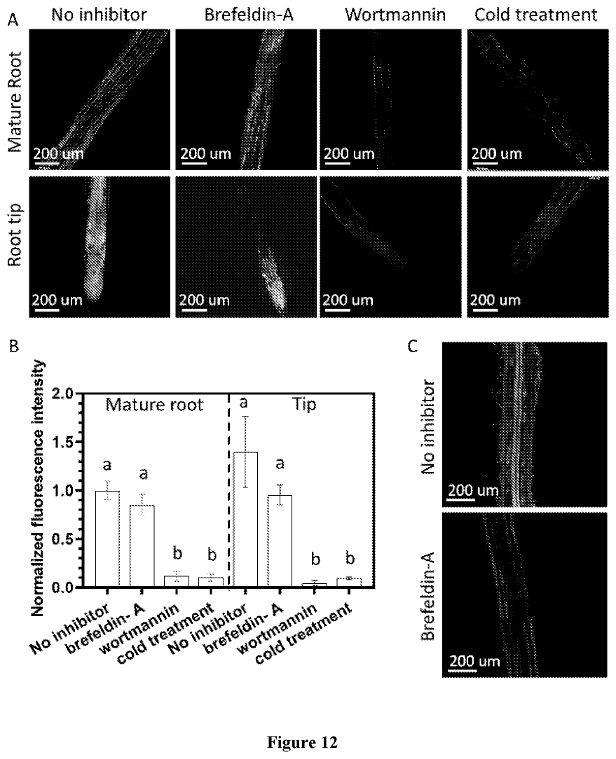

Brefeldin-A is an inhibitor of intracellular sorting and transport of vesicles from the ER to the Golgi complex, and treatment with this inhibitor results in an accumulation of proteins in the ER, and the loss of exocytosis and the protein secretion pathway in eukaryotic cells. Pre-treatment of plant tissue with Brefeldin-A did not prevent uptake of the lysozyme-coated nanoparticles by the root tip and epidermal cells of mature roots and had only a slight and insignificant impact on the fluorescence intensity in the root tip and epidermal cells (FIG. 12A-C). However, pre-treatment with Brefeldin did result in a striking loss of fluorescence in all other parts of the root, including an attenuated fluorescent signal in the cell elongation zone immediately above the root tip (FIG. 12A), and also in the cortex and stele of mature roots (FIG. 12C). Furthermore, bright fluorescent foci were observed in the root tip and epidermal cells after treatment with Brefeldin-A (FIG. 12A). These fluorescent foci are most likely due to the Lys@LDH30-FL nanoparticles accumulating and complexing within ER of the root tip and epidermal cells.

Remarkably, pre-treatment with the endocytosis inhibitor Wortmannin and/or at cold temperatures effectively eliminated the uptake and cellular internalization of lysozyme-coated nanoparticles by the roots (FIG. 12A) In this regard, the fluorescence intensity of the Wortmannin or cold temperature pre-treated mature roots and root tips was ≤10% of the ‘no inhibitor’ control roots (FIG. 12B). These data demonstrate that energy-dependent, receptor-mediated endocytosis is required for the uptake, cellular internalization and translocation of the lysozyme-coated nanoparticles by the roots.

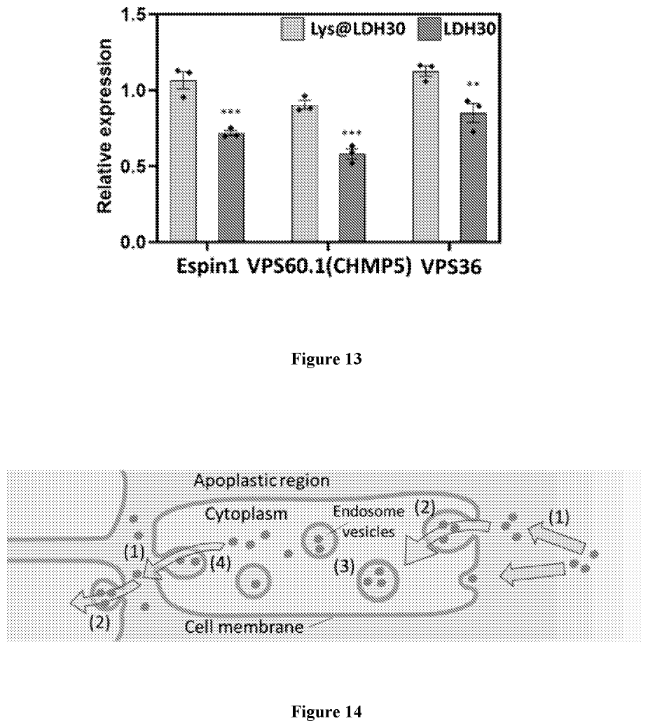

To further investigate how nanoparticles and lysozyme coating affect with endocytosis and vesicle trafficking, the inventors have analysed the expression of a collection of conserved genes related to endocytosis. EPSINI plays an important role in the trafficking of regulatory proteins across the trans-Golgi network via an interaction with clathrin, whereas VPS60.1 (the Arabidopsis orthologue of CHMP5) and VPS36 are involved in intracellular vesicle trafficking. Sorting and/or exocytosis treatment of Arabidopsis seedlings with lysozyme-coated LDH30 nanoparticles significantly enhanced the expression of these three endocytosis-related genes in the roots compared to treatment with uncoated LDH30 nanoparticles (FIG. 13). These results suggest that stimulation of endocytosis and the membrane trafficking pathway may have contributed to the enhanced uptake of lysozyme-coated LDH30 nanoparticles compared to the uncoated LDH nanoparticles.

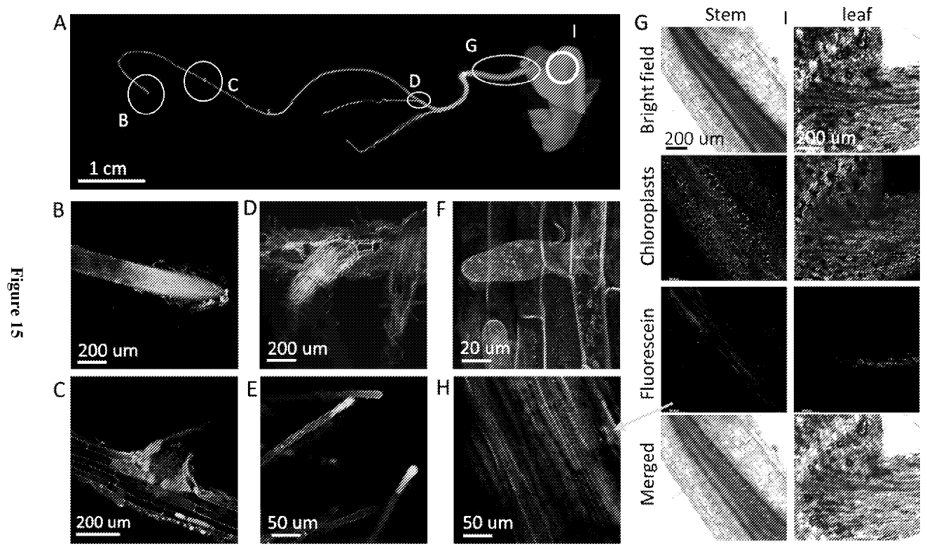

Collectively, these results clearly indicate that the lysozyme-coated nanoparticles are taken up into the root tip and epidermal cells by energy-dependent, receptor-mediated endocytosis, and that the subsequent translocation of the nanoparticles to other cell types requires the membrane trafficking pathway, involving repeated cycles of endocytosis, intracellular vesicle transport, exocytosis and apoplastic transport, as schematically illustrated in FIG. 14.

### Sites for Nanoparticle Internalization and Translocation

As mentioned previously, the root tip and cell elongation zone appeared to take up more nanoparticles compared to the mature root. To locate sites for the uptake and translocation of lysozyme-coated nanoparticles further, the inventors used a blue light-excited fluorescence imaging system and confocal microscopy. Several sites in the root and hypocotyl showed much brighter fluorescence than the surrounding tissues, as indicated by the circles and capital letters in FIG. 15A. The fluorescence intensity of the root tip (from the root cap to the cell elongation zone) was substantially higher than the other parts of the root (FIG. 15B).

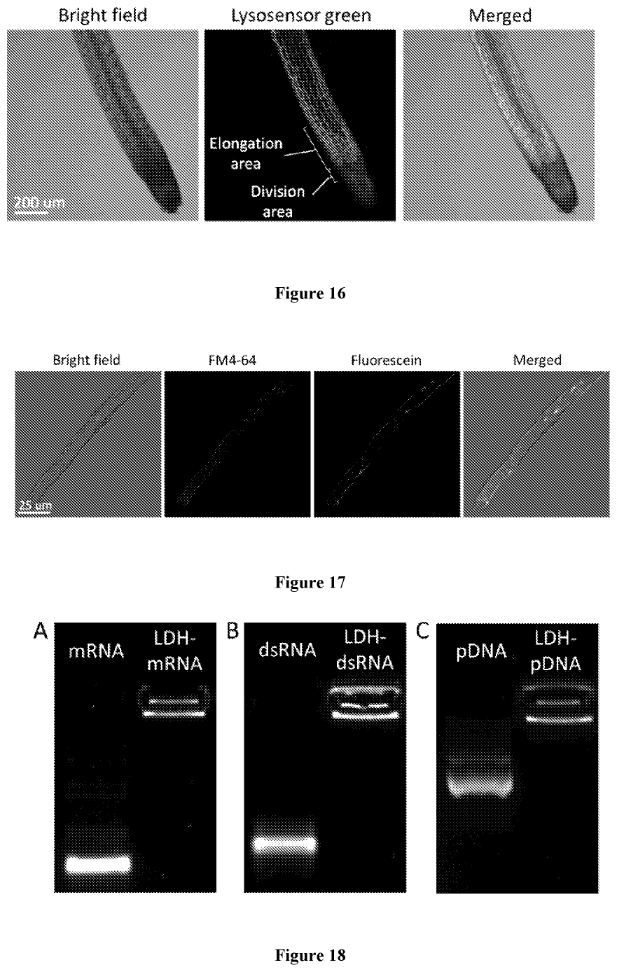

The inventors also applied the pH probe Lysosenser Green to visualize variation in pH within the developing root. The strongest fluorescent signal was observed in the zone of cell expansion immediately above the region of rapidly dividing cells at the root tip (FIG. 16). This strong fluorescence signal indicated a lower pH due to the efflux of protons from the cytoplasm and a loosening of the cell wall to allow cell expansion in the elongation zone to occur. Collectively, these observations demonstrate that the uptake of lysozyme-coated nanoparticles was greatest in the region of cell division where the primary cell wall is very thin, and in the zone of cell expansion where a lower pH loosens the cell wall to facilitate cell expansion.

Besides the root tip, some other sites in the root showed brighter fluorescein fluorescence than the surrounding tissues (FIG. 15A-F). In this regard, the lysozyme-coated nanoparticles were also taken up and internalized in the junctions of emerging lateral root (FIG. 15C), as well as in the cortex and vascular cylinder of mature lateral roots where they join onto the main root (FIG. 15D). The third hot-spot for uptake and internalization of lysozyme-coated nanoparticles was root hairs, particularly in the growing tip of root hairs where considerable endocytosis and vesicle trafficking are known to occur (FIGS. 15E and F). Under a higher magnification (FIG. 15F), brighter nanoparticle fluorescence was observed in sphere-like foci in the growing tip of root hairs. These small foci can co-localize with the endocytosis-related vesicles stained with membrane-specific dye FM4-64 (FIG. 17), suggesting that the fluorescence most likely represent lysozyme-coated nanoparticle-containing vesicles formed during endocytosis.

Although much weaker compared to the signals in the root, lysozyme-coated nanoparticle fluorescence was also observed in the stem and petioles of the seedlings (FIG. 15A). These signals were mainly located within the vascular bundles and surrounding cells (FIG. 15G). A distinct pattern of xylem tracheary element structures (FIG. 15H) was observed in terms of the fluorescein signal distribution, suggesting that translocation of lysozyme-coated nanoparticles from root to shoot occurs mainly via the xylem. The fluorescence signals were also observed in young leaves (FIG. 15G), especially petiole cells and veins close to the base of the developing leaf blade. These data indicate that a portion of the lysozyme-coated nanoparticles translocate from the roots to cotyledons and young leaves within 4 h post incubation. Furthermore, consistent with the upward translocation of lysozyme-coated nanoparticles from the roots to shoot tissues, increased fluorescein signals were also observed in the upper part of the root at 4 h post incubation.

Delivery of Functional mRNA into Root Cells

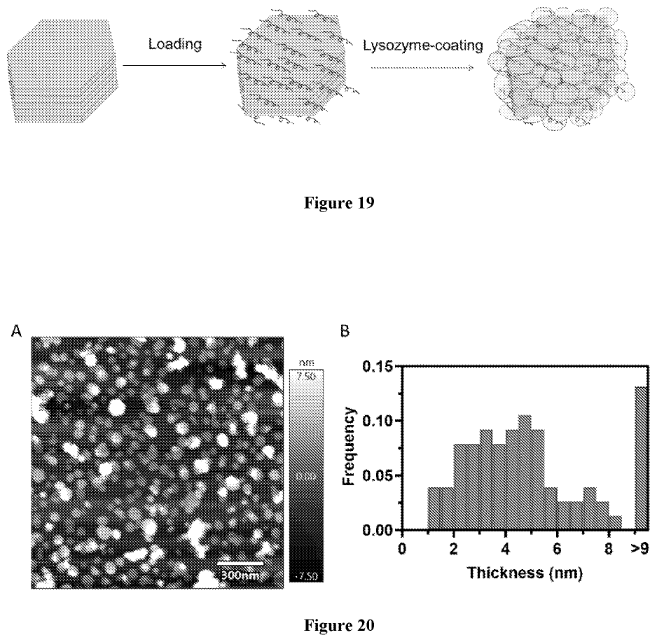

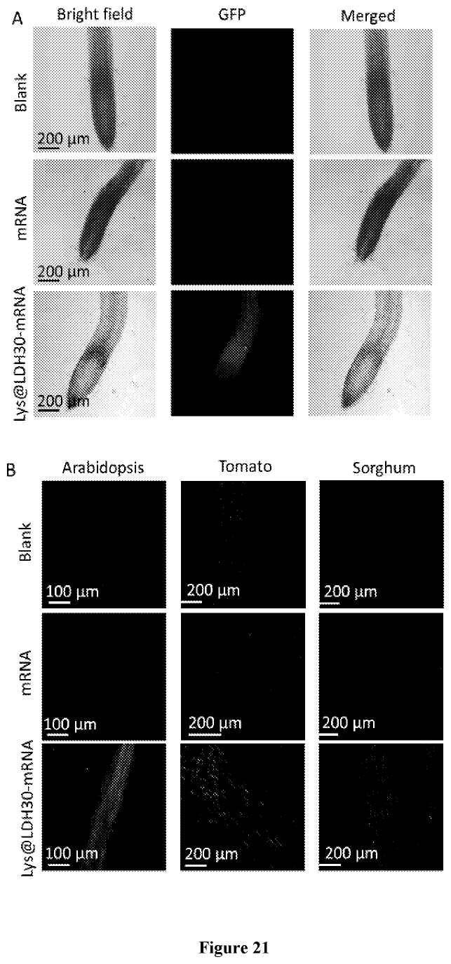

To test if the lysozyme-coated LDH nanocarriers could be used to deliver mRNA into root cells for translation into functional protein, LDH30 nanoparticles were loaded with mRNA encoding green fluorescence protein (GFP; Table 3; FIG. 19). After the mRNA was loaded onto nanoparticles (FIG. 18A), the Lys@LDH30-mRNA nanoparticles showed very slight aggregation and the average thickness was increased to ~5.0 nm (FIG. 2A; FIGS. 20A and B).

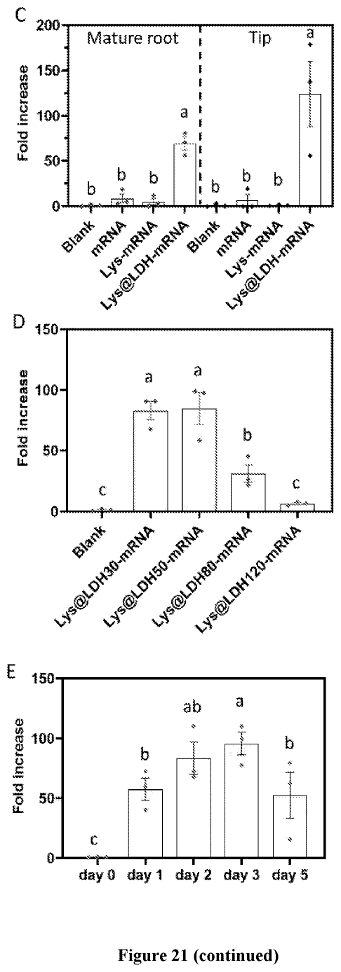

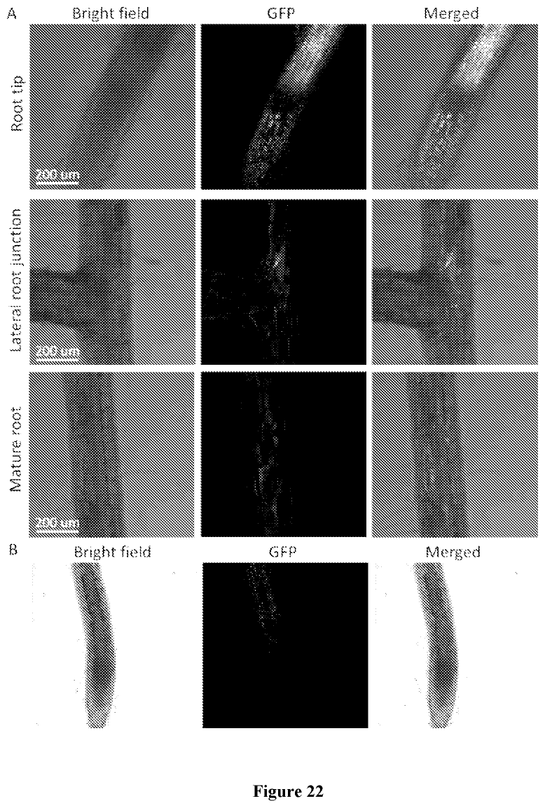

After 2 days incubation with the Lys@LDH30-mRNA nanoparticles, strong GFP fluorescence was observed in the root in comparison with the naked mRNA treatment and the blank control (FIGS. 21 and 22). The inventors also observed a slightly stronger GFP signal in the cell elongation zone and the area around the lateral root junctions (FIG. 22A), which is consistent with more nanoparticles being internalized in these regions of the root (FIGS. 15C and D). Quantitative analysis on N. benthamiana roots revealed that both the root tip and mature root sections showed GFP fluorescence at ~200 and >100 times the level of background autofluorescence, respectively, at 2 days post treatment with lysozyme-coated LDH30-mRNA (FIG. 21C). Weak GFP fluorescence was occasionally detected in N. benthamiana roots 2 days after treatment with naked mRNA, albeit very weakly and only in epidermal cells (FIG. 22B) suggesting that a very low amount of naked mRNA was taken up and translated in the root epidermis. Combining lysozyme with mRNA in the absence of LDH nanoparticles resulted in the formation of complexes that failed to migrate through the gel when subjected to electrophoresis (FIG. 24), and therefore not surprisingly, the roots treated with this complex showed an even weaker GFP fluorescence compared to the naked mRNA-treated roots (FIG. 21C). These observations clearly demonstrate that lysozyme-coated nanoparticles are an effective vector for delivering in vitro-synthesized mRNA into N. benthamiana roots for translation into functional protein.

TABLE 3
Sequence of GFP mRNA.

| | |
|---|---|
| GFP mRNA | ACUCUUCUGGUCCCCACAGACUCAGAGAGAACCCGCC |
| (1184-nt, | ACCAUGGUGAGCAAGGGCGAGGAGCUGUUCACCGGGG |
| 5′→3′) | UGGUGCCCAUCCUGGUCGAGCUGGACGGCGACGUAAA |
| (SEQ ID | CGGCCACAAGUUCAGCGUGUCCGGCGAGGGCGAGGGC |
| NO: 1) | GAUGCCACCUACGGCAAGCUGACCCUGAAGUUCAUCU |
| | GCACCACCGGCAAGCUGCCCGUGCCCUGGCCCACCCUC |
| | GUGACCACCCUGACCUACGGCGUGCAGUGCUUCAGCC |
| | GCUACCCCGACCACAUGAAGCAGCACGACUUCUUCAA |
| | GUCCGCCAUGCCCGAAGGCUACGUCCAGGAGCGCACC |
| | AUCUUCUUCAAGGACGACGGCAACUACAAGACCCGCG |
| | CCGAGGUGAAGUUCGAGGGCGACACCCUGGUGAACCG |
| | CAUCGAGCUGAAGGGCAUCGACUUCAAGGAGGACGGC |
| | AACAUCCUGGGGCACAAGCUGGAGUACAACUACAACA |
| | GCCACAACGUCUAUAUCAUGGCCGACAAGCAGAAGAA |
| | CGGCAUCAAGGUGAACUUCAAGAUCCGCCACAACAUC |
| | GAGGACGGCAGCGUGCAGCUCGCCGACCACUACCAGC |
| | AGAACACCCCCAUCGGCGACGGCCCCGUGCUGCUGCCC |
| | GACAACCACUACCUGAGCACCCAGUCCGCCCUGAGCAA |
| | AGACCCCAACGAGAAGCGCGAUCACAUGGUCCUGCUG |
| | GAGUUCGUGACCGCCGCCGGGAUCACUCUCGGCAUGG |
| | ACGAGCUGUACAAGUAAUAACUCGAGCUGGUACUGCA |
| | UGCACGCAAUGCUAGCUGCCCCUUUCCCGUCCUGGGU |
| | ACCCCGAGUCUCCCCCGACCUCGGGUCCCAGGUAUGCU |
| | CCCACCUCCACCUGCCCCACUCACCACCUCUGCUAGUU |
| | CCAGACACCUCCCAAGCACGCAGCAAUGCAGCUCAAA |
| | ACGCUUAGCCUAGCCACACCCCCACGGGAAACAGCAG |
| | UGAUUAACCUUUAGCAAUAAACGAAAGUUUAACUAAG |
| | CUAUACUAACCCCAGGGUUGGUCAAUUUCGUGCCAGC |
| | CACACCCUGGAGCUAGCAAAAAAAAAAAAAAAAAAAA |
| | AAAAAAAAAAAAAAAAAAAAAAAAAAAAAAAAAAAA |
| | AAAAAUGCAUAAAAAAAAAAAAAAAAAAAAAAAAAA |
| | AAAAAAAAAAAAAAAAAAAAAAAAAAAAAAAAAA |

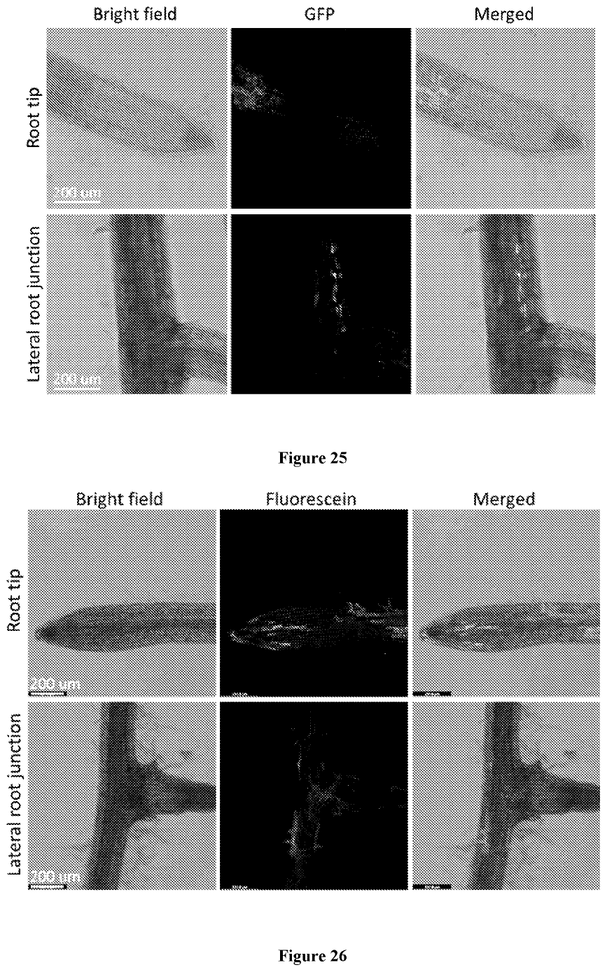

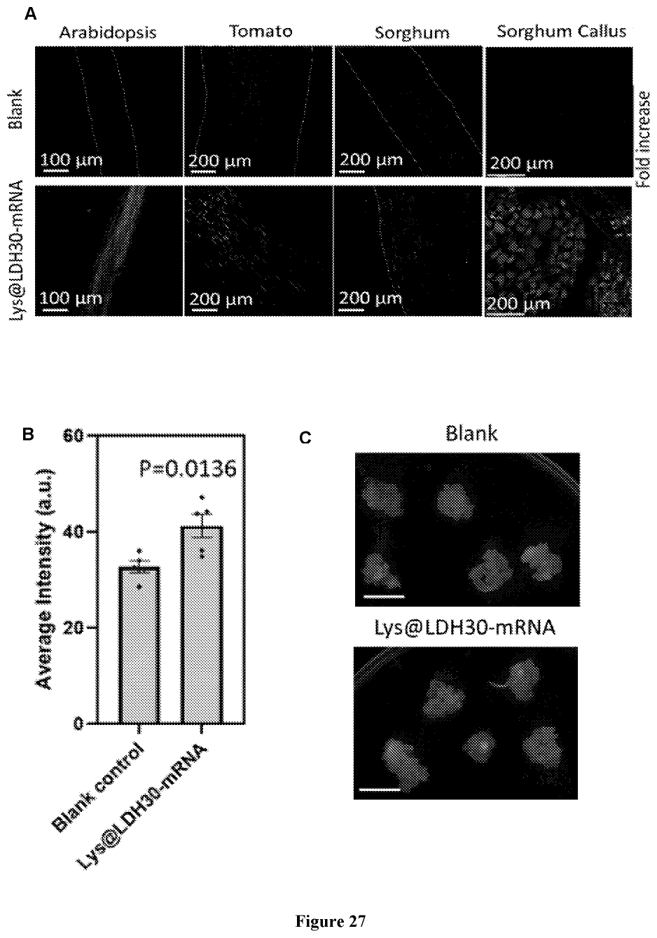

The efficacy of GFP mRNA delivery into mature N. benthamiana roots was remarkably affected by the size of lysozyme-coated nanoparticles, which the inventors tested in the range of 30 to 120 nm (FIG. 21D). Lys@LDH30-mRNA and Lys@LDH50-mRNA treated mature roots showed the highest GFP fluorescence (85-90 times the background autofluorescence; FIG. 21D), whereas the GFP fluorescence intensity of mature roots incubated with Lys@LDH80-mRNA was significantly lower (32.7 times the background fluorescence; FIG. 21D). The Lys@LDH120-mRNA treatment resulted in an even lower levels of GFP in mature roots, with only a 6.9-fold increase in the GFP fluorescence intensity compared to the untreated control (FIG. 21D), where GFP fluorescence was only detected in some expanding epidermal cells in the root elongation zone and in cortex cells near lateral root junctions (FIG. 25), which is consistent with limited internalization of Lys@LDH120-FL nanoparticles in these regions of the root (FIG. 26).

The inventors further investigated the time-course of GFP expression in mature N. benthamiana roots following the addition of Lys@LDH30-mRNA nanoparticles to culture media. Interestingly, there was significant expression of GFP at day 1 after treatment, which continued to increase until day 3 post incubation (FIG. 21E). Subsequently, by day 5 post treatment, the level of GFP fluorescence intensity decreased to the similar level observed at day 1 (FIG. 21E). The eventual decline in GFP levels by day 5 likely reflects the natural half-life of the GFP mRNA and protein in root cells. Nevertheless, this time-dependent profile demonstrates transient expression of the mRNA-encoded GFP protein over about 5-7 days post treatment with a single dose of lysozyme-coated LDH nanoparticles.

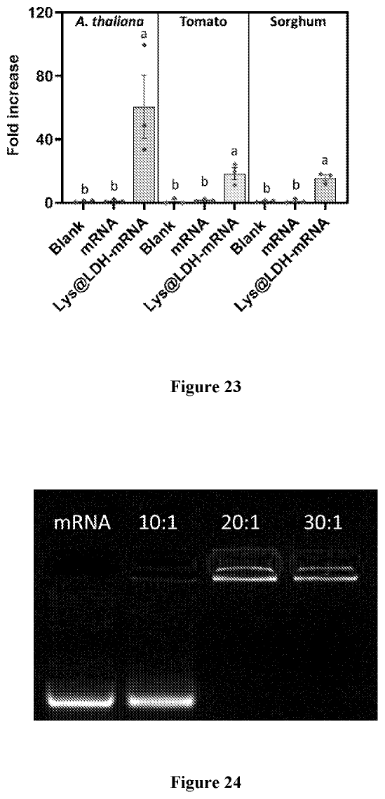

In addition to N. benthamiana, the inventors used lysozyme-coated LDH30 nanoparticles to deliver in vitro-synthesized GFP mRNA into the roots of Arabidopsis thaliana (Col-0), tomato (Solanum lycopersicum, commercial variety Moneymaker) and sorghum (Sorghum bicolor, inbred line 430R). As illustrated in FIG. 21B, GFP fluorescence was clearly observed in mature roots of all three plant species at 2 days post incubation with Lys@LDH30-mRNA. The GFP fluorescence intensity was approximately 60-, 18- and 15-fold higher than background autofluorescence for Arabidopsis, tomato, and sorghum mature roots, respectively (FIG. 23). Besides roots, the lysozyme-coated LDH30 also delivered mRNA to sorghum callus through simple co-incubation. The expressed GFP signal was found within the cell cytosol in the calli slice (FIG. 27A) at 2 days post incubation with lysozyme-coated LDH30-mRNA. Strong GFP fluorescence, with an over 25% increase to the auto-fluorescence, was observed in callus under blue light handlamp excitation compared to blank control group (FIGS. 27B and C). Collectively, these data clearly demonstrate that the lysozyme-coated LDH30 delivery platform can be used to deliver in vitro-synthesized mRNA into dicot and monocot crop species.

Delivery of dsRNA, siRNA and pDNA to Plant Roots

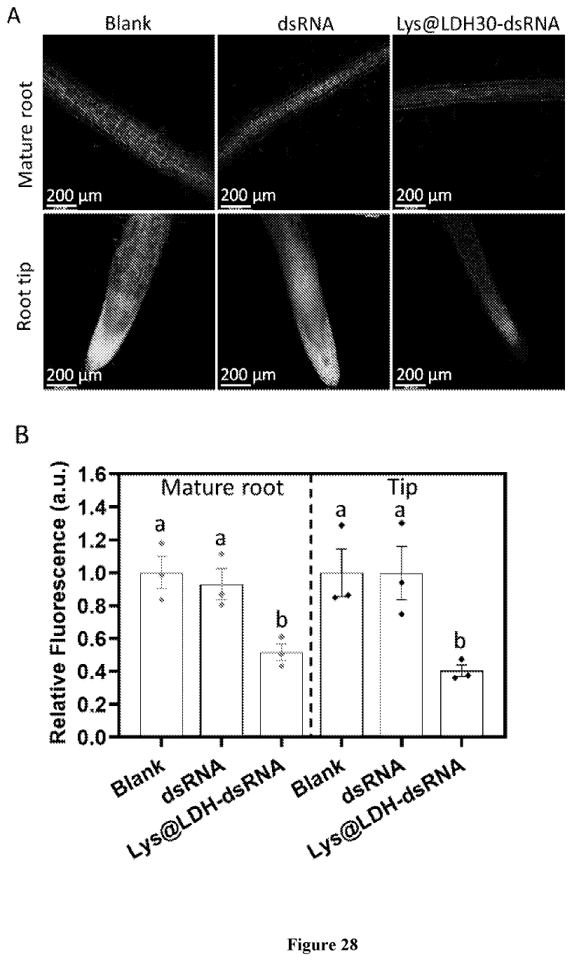

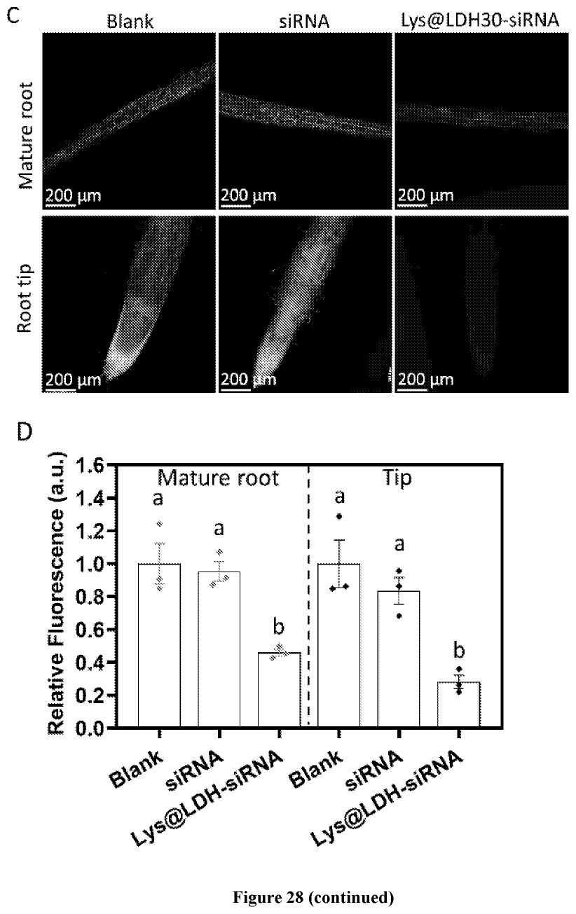

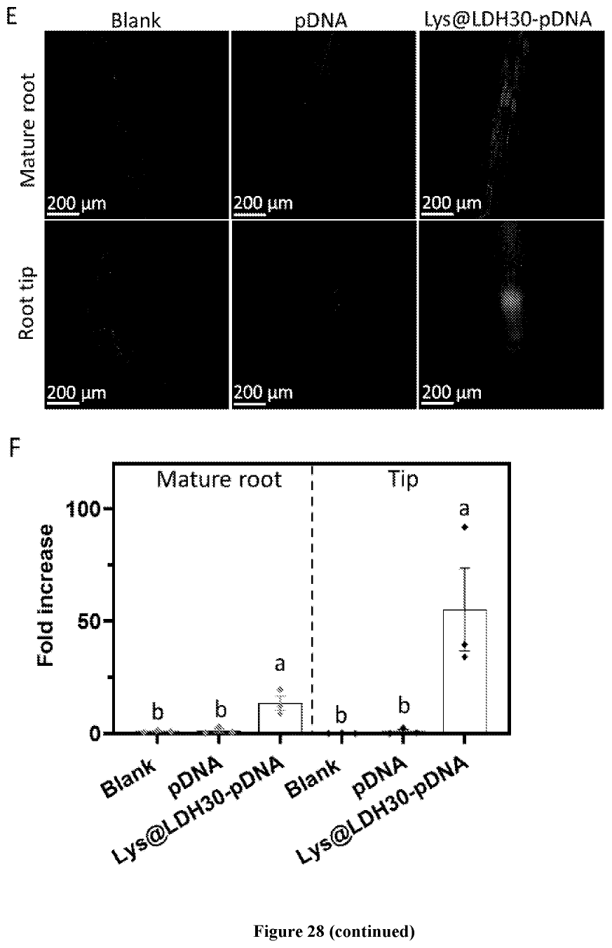

In addition to delivering in vitro-synthesized mRNA into roots and calli, the inventors have also successfully used lysozyme-coated LDH30 nanoparticles to deliver a 415-bp double-stranded RNA (dsRNA, Table 4, FIGS. 28A and B) and small interfering RNA (siRNA, Table 4; FIGS. 28C and D) and induce RNAi of GFP in the transgenic N. benthamiana line 16c.

TABLE 4
Sequence of GFP dsRNA and siRNA employed.

| GFP dsRNA | GGGTAAAGGAGAAGAACTTTTCACTGGAGTTGTCCCAA |
|---|---|
| (415-bp, | TTCTTGTTGAATTAGATGGTGATGTTAATGGGCACAAA |
| 5′→3′) | TTTTCTGTCAGTGGAGAGGGTGAAGGTGATGCAACATA |
| (SEQ ID | CGGAAAACTTACCCTTAAATTTATTTGCACTACTGGAA |
| NO: 2) | AACTACCTGTTCCATGGCCAACACTTGTCACTACTTTC |
| | TCTTATGGTGTTCAATGCTTTTCAAGATACCCAGATCA |
| | TATGAAGCGGCACGACTTCTTCAAGAGCGCCATGCCTG |
| | AGGGATACGTGCAGGAGAGGACCATCTTCTTCAAGGAC |
| | GACGGGAACTACAAGACACGTGCTGAAGTCAAGTTTGA |
| | GGGAGACACCCTCGTCAACAGGATCGAGCTTAAGGGAA |
| | TCGATTTCAAGGAGGCCCTATAGTGAGTCGTATTA |
| GFP siRNA | UUC CGU AUG UUG CAU CAC CTT |
| antisense | (SEQ ID NO: 3) |
| (21-nt, | |
| 5′→3′) | |
| GFP siRNA | GGU GAU GCA ACA UAC GGA ATT |
| sense | (SEQ ID NO: 4) |
| (21-nt, | |
| 5′→3′) | |

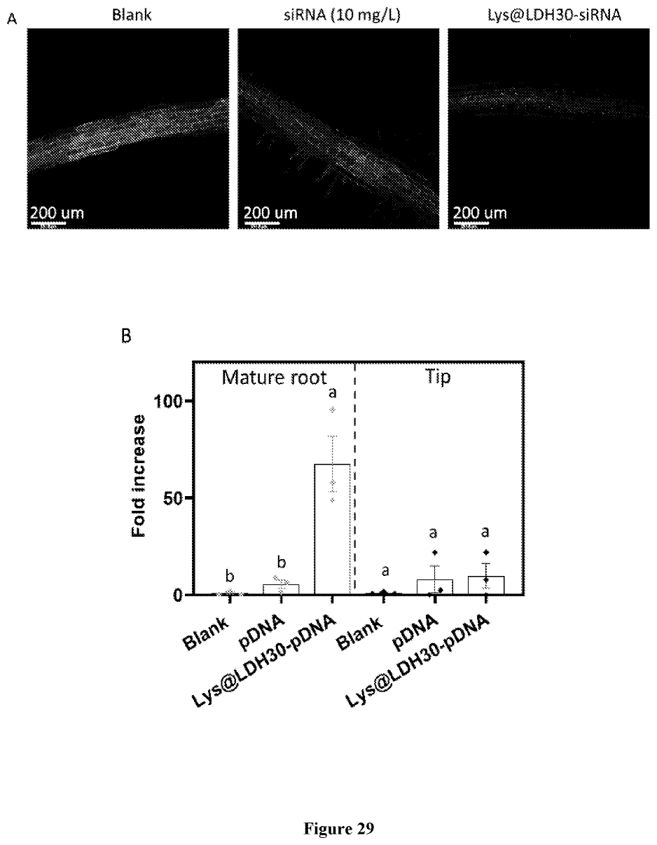

Roots incubated with lysozyme-coated LDH30-dsRNA (Lys@LDH30-dsRNA) at the single dsRNA dose of 10 mg/L for 3 days showed a 49% and 59% decrease of the GFP fluorescence in mature roots and root tips, respectively (FIGS. 28A and B). This level of GFP silencing was significantly higher than the <10% decrease in the GFP fluorescence induced by treating the roots for 3 days with the same concentration of naked dsRNA (FIGS. 28A and B). Similarly, the GFP intensity in the mature root and root tips was decreased by 54% and 72%, respectively, after a 3-day incubation with a single dose of lysozyme-coated LDH30-siRNA (Lys@LDH30-siRNA) at the siRNA concentration of 5 mg/L (FIGS. 28C and D). In contrast, a single dose of naked siRNA caused only an insignificant 5% and 16% decrease in GFP florescence for mature roots and root tips, respectively (FIGS. 28C and D). These data thus demonstrate that lysozyme-coated LDH30 nanoparticles effectively facilitates internalization of dsRNA and siRNA by root cells to induce silencing of the homologous target gene. It is worth noting that when the siRNA concentration was doubled, the naked siRNA induced a significant decrease of GFP (by 29%) compared to blank group at 3 days post incubation (FIG. 29). This observation confirms that naked siRNA was also internalized by root cells to induce RNAi, however, the level of RNAi induced was greatly enhanced by using the Lys@LDH30 nanoparticles to deliver the siRNA (FIG. 28).

The inventors then demonstrated the successful use lysozyme-coated LDH30 nanoparticles to deliver transgene-containing plasmid DNA to the transgenic N. benthamiana line 16c, and in so doing express functional transgenes within the plasmid in N. benthamianai tissue.

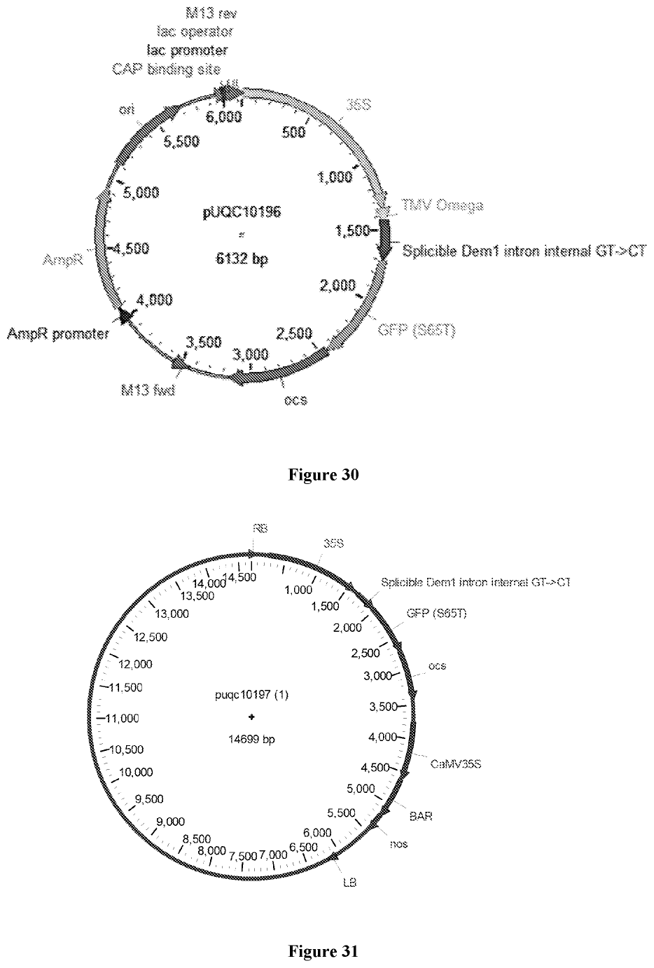

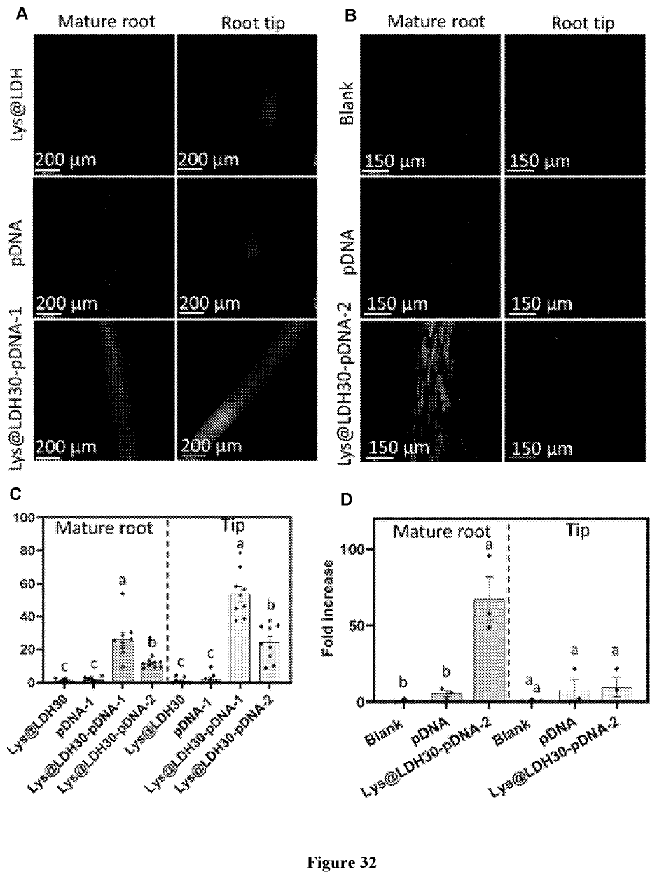

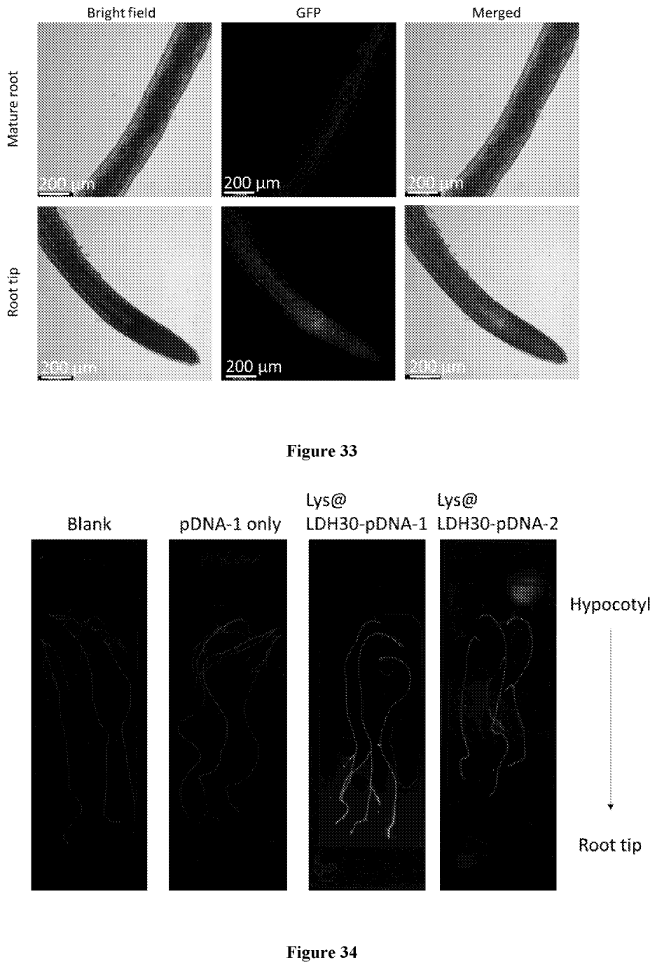

In one experiment, the inventors loaded a 6 kb plasmid encoding GFP driven by a 35S promoter (pDNA-1, FIG. 30), and its 14 kb analogue with the same promoter and GFP sequence (pDNA-2, FIG. 31) onto the lysozyme-coated LDH30 nanoparticles to demonstrate the efficacy of plasmid delivery into N. benthamiana roots for expression of the functional transgene (FIG. 32A). Expression of the GFP protein was observed in both root tips (~53 times the background fluorescence) and mature roots (~26 times the background fluorescence) at 2 days post incubation with lysozyme-coated LDH30-pDNA-1, which is in contrast to the negligible fluorescence observed for the untreated and plasmid only control roots (FIGS. 32A and C; and FIG. 34). Meanwhile, although loading of a plasmid as large as 14.7 kb is trickier than the small plasmid, the successful delivery of such large vector is confirmed by the detection of GFP fluorescence under confocal microscope (FIGS. 32B and D; FIG. 33; and FIG. 34)

Similarly, the inventors loaded a 5839-bp plasmid encoding YFP with 35S promoter onto lysozyme-coated LDH30 nanoparticles (Lys@LDH30-pDNA) for delivery to N. benthamiana roots. Expression of YFP protein was observed in both mature roots (13 times the background fluorescence) and root tips (55 times the background fluorescence) at 2 days post incubation with Lys@LDH30-pDNA, comparing to negligible fluorescence of blank control and plasmid only treated roots (FIG. 28E-F).

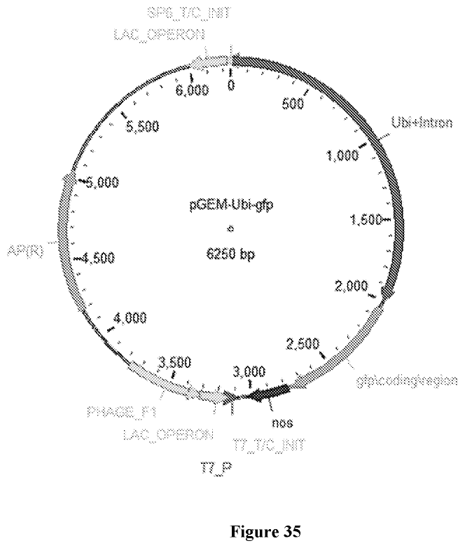

To further demonstrate the versatility of the lysozyme-coated nanoparticles for delivery of a transgene into the roots of a monocot species, a 6250-bp plasmid encoding GFP transgene driven by a constitutive Ubi promoter (FIG. 35) was also loaded onto lysozyme-coated LDH30 nanoparticles (Lys@LDH30-pDNA-3) to treat the roots of 7-day-old 430R sorghum seedlings. Strong GFP fluorescence (>60 times the background autofluorescence) was observed in the epidermal cells of the sorghum roots at 2 days post incubation with Lys@LDH30-pDNA at a single dose of 2 mg/L DNA. Minimal GFP expression was evident in roots treated with naked plasmid DNA. However, GFP expression was not detected in the zone of cell elongation near the root tip of sorghum roots treated with Lys@LDH30-pDNA nanoparticles, suggesting either some variation in the pattern of nanoparticle uptake between different plant species and/or differences in the expression patterns of the 35S and Ubi promoters (see FIGS. 32B and D).

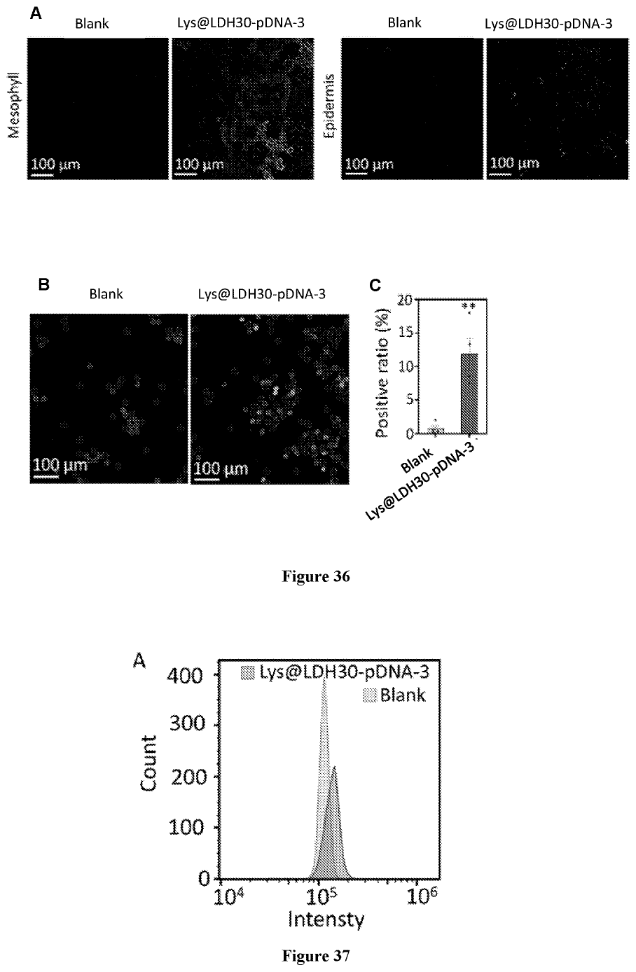

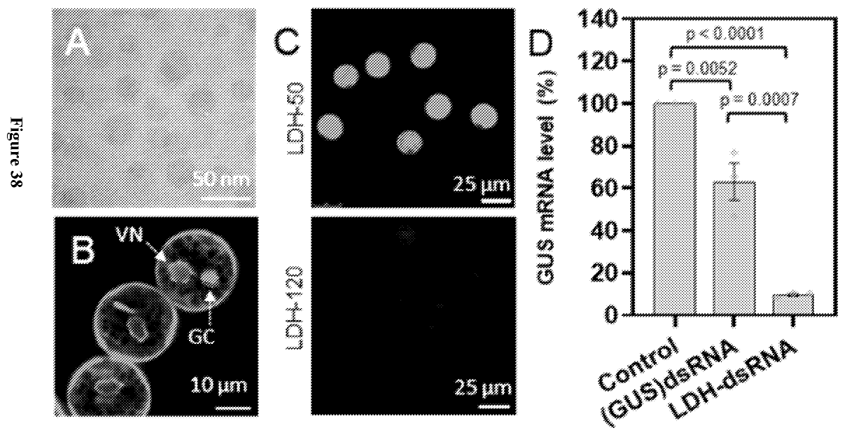

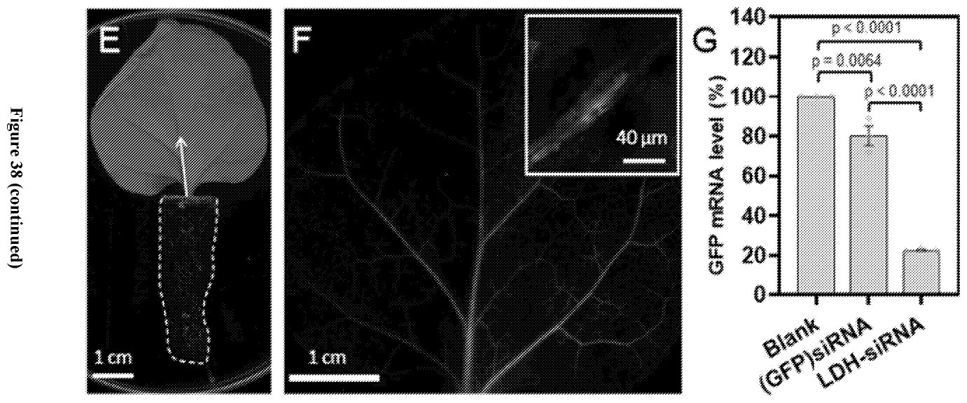

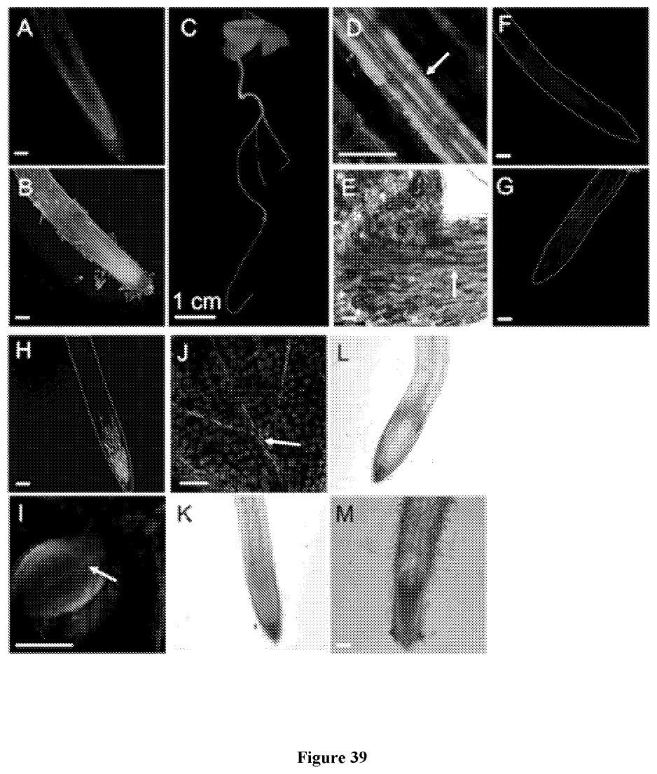

Finally, to further demonstrate the range of different applications in which lysozyme-coated LDH30 nanoparticles can be used to deliver exogenous genes into plants, the inventors delivered the pDNA-3 (FIG. 35) into N. benthamiana leaves by infiltration and into developing tomato pollen by flower bud injection. As observed above for root tissue, GFP fluorescence was observed in both mesophyll and epidermal cells of tobacco leaves infiltrated with lysozyme-coated LDH30-pDNA-3 (FIG. 36A). The inventors also injected the lysozyme-coated LDH30-pDNA-3 into the anther region of developing tomato flower buds (4-6 mm) and demonstrated that more than 10% of pollen extracted from the flower buds showed GFP expression at 3 days post injection (FIGS. 36B and C; FIG. 37). The relatively low frequency of GFP expression in the developing pollen could be attributed to a limited infiltration of the nanoparticle solution throughout the flower bud and/or the low activity of the 35S promoter in the developing pollen. Nevertheless, these results clearly demonstrate the wide applicability of the lysozyme-coated LDH30 nanoparticles as a universal platform for the effective delivery of functional RNA and transgenes into plants.

### Conclusion

Collectively, the data presented in Example 1 demonstrate that the inventors have developed a highly efficient and universal platform based on lysozyme-coated LDH30 nanoparticles for delivery of functional RNA and transgenes into plants and plant tissues, including dicot and monocot crop species.

### Example 2: LDH Nanoparticles can Traverse the Plant Cell Wall and Facilitate the Uptake of dsRNA and siRNA by Plant Cells to Induce RNAi

A natural plant defence mechanism against viruses in plants is RNA interference (RNAi), also known as RNA silencing. There is growing evidence that this mechanism plays an important role in natural plant defences against parasites, viruses, insects, nematodes and fungal infections, as well as transposon activity. Through this mechanism exogenous or endogenous double-stranded RNA (dsRNA) is diced into small interfering RNA (siRNA), which is then incorporated into an RNA-induced silencing complex (RISC). The active RISC then uses siRNA to detect and degrade targeted viral RNA, thereby giving rise to antiviral defence.

The inventors demonstrated that gene silencing induced by exogenous application of long dsRNA, siRNA duplexes and mature amiRNAs can be delivered using nanoparticles. Layered double hydroxide (LDH) nanoparticles can measure from 15 to 120 nm in diameter (FIG. 38A) and while the cell wall is a challenging barrier to nanoparticle delivery into plant cells, the inventors have demonstrated that unlike the larger LDH, smaller LDH nanoparticles up to 50 nm in diameter (LDH-50) can traverse through the plant cell wall and facilitate the uptake of dsRNA and siRNA by plant cells to induce RNAi.

### Materials and Methods

The materials and methods were the same as used in Example 1, with the exception of the following details.

### Leaf Infiltration

The LDH nanoparticles loaded with siRNAs (FIG. 38G) were suspended in 20 mM MES pH 6.0 buffer. Using a 1 ml needleless syringe, 50~100 μl of the LDH nanoparticle suspension was slowly infiltrated through the abaxial leaf surface of 5-7 week-old tobacco plants. Tissue paper was used to gently remove any residual liquid on the leaf surface. The plants were then kept in the growth room until the infiltrated leaf tissue was harvested for analysis.

### Petiole Application on Excised Leaf

For the excised leaf application, the leaf petiole was cut 0.5 cm from the leaf blade and immersed into a 1.5 mL microfuge tube containing 20 mM MES buffer (pH 6.0) supplemented with 200 mg/L of LDH-FITC (FIG. 38E-F). The tube was capped with parafilm to prevent evaporation and covered with aluminum foil to prevent photobleaching of FITC.

### Results

The inventors used developing tomato pollen as a model system to demonstrate that LDH50, but not larger nanoparticles, are readily internalised (FIG. 38C) and facilitate the uptake of long dsRNAs and the almost complete silencing of the targeted GUS reporter gene (FIG. 38D). The inventors also showed that LDH50 nanoparticles can translocate along the vasculature of excised, petiole-infiltrated tobacco leaves (FIG. 38E-F), and internalise into mesophyll cells of infiltrated intact leaves to facilitate the uptake of siRNA duplexes to direct efficient silencing of the GFP reporter gene (FIG. 38G).

### Conclusion

LDH nanoparticles of up to 50 nm in diameter are able to traverse the plant cell wall to facilitate uptake of nucleic acids.

### Example 3: Lysozyme-Coated LDH50 Nanoparticles for Active Uptake, Translocation and Delivery of Functional Nucleic Acids into Plants

The inventors have designed nanoparticles for active uptake, translocation and delivery of nucleic acids into plants for non-GM, somatic or heritable gene editing and genome manipulation. They demonstrated a significantly increased uptake of LDH50 nanoparticles and enhanced nanoparticle translocation throughout the vasculature of the whole plant when these nanoparticles were coated in protein.

### Materials and Methods

The materials and methods were the same as used in Example 1, with the exception of the following details.

### Excised Shoot Application

7-day-old Arabidopsis shoots were excised just above the base of the hyocotyl, immediately above shoot-root junction. The hypocotyl of the excised shoot was incubated in a 96-well-plate containing 20 mM MES buffer (pH 6.0) supplemented with 200 mg/L of lysozyme-coated LDH50-fluorescein.

### Results

The inventors showed that LDH nanoparticles<50 nm in diameter (LDH50) can be taken up from hydroponic growth medium by roots of whole seedlings and also excised shoots (FIG. 39A). Further encapsulating LDH50 nanoparticles in lysozyme protein resulted in a greatly increased uptake of the LDH50 nanoparticles by the roots (compare FIGS. 39A and B). The protein coating also enhanced nanoparticle translocation throughout the vasculature of the whole plants (FIG. 39C-E). Pre-treatment of roots with the endocytosis inhibitor, Wortmannin (FIG. 39F), or cold treatment (FIG. 39G) completely prevented the uptake and cellular internalisation of lysozyme-coated LDH50 nanoparticles. In contrast, pre-treatment with Brefeldin-A, an inhibitor of intra-cellular vesicle transport and exocytosis, had almost no impact on uptake of lysozyme-coated LDH50 nanoparticles by cells in the root tip but prevented translocation of the nanoparticles to other parts of the root (FIG. 39H). These results demonstrated that lysozyme-coated LDH50 nanoparticles are actively taken up via endocytosis and translocate through the roots via the membrane trafficking pathway into the vasculature of the plant. The inventors also detected LDH50 nanoparticles in the vasculature of leaf primordia and developing leaves after incubation of excised Arabidopsis shoots in hydroponic growth media containing the lysozyme-coated nanoparticles (FIG. 39I-J). Uncoated LDH50 nanoparticles facilitated the delivery of functional GFP mRNA (FIG. 39K), however, GFP florescence was greatly enhanced by coating the nanoparticles in lysozyme (FIG. 39L). Treatment of roots with lysozyme-coated LDH50 nanoparticles encoding an intron-spliceable 35S:GFP transgene also resulted in high levels of GFP florescence in the roots (FIG. 39M).

### Conclusion

Encapsulating LDH50 nanoparticles in lysozyme protein resulted in a greatly increased uptake of the LDH50 nanoparticles and enhanced nanoparticle translocation throughout the vasculature of the whole plant as compared to uncoated LDH50 nanoparticles.

### Example 4:—Delivery of Transgene Fragments Via Lysozyme-Coated LDH Nanoparticles Induces Increased Transgene Expression in Plant Tissue

To further demonstrate the versatility of the lysozyme-coated nanoparticle delivery system, the inventors undertook further experiments in which lysozyme-coated LDH30 nanoparticles were used to deliver a PCR-amplified transgene fragment to the transgenic N. benthamiana line 16c.

### Materials and Methods

The materials and methods were the same as used in Example 1, with the exception of the following details.

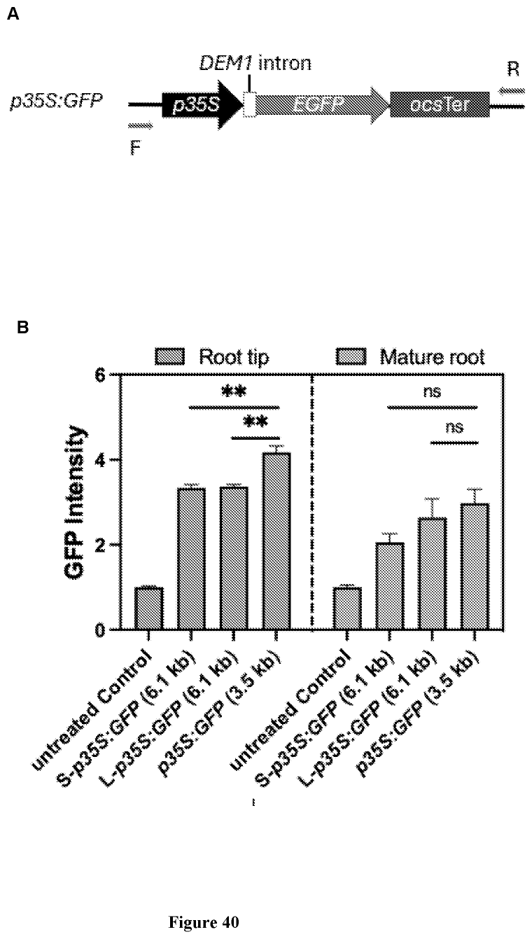

Using PCR, the inventors amplified a 3.5 kb transgene fragment of the pDNA-1 plasmid (described in Example 1) comprising, in a 5′ to 3′ direction, the p35S promoter, the eGFP transgene and ocs-element (′p35S:GFP′; FIG. 40A). The inventors then loaded lysozyme-coated LDH30 nanoparticles with the p35S:GFP transgene, supercoiled pDNA-1 plasmid (S-p35S:GFP) or linearised pDNA-1 plasmid (L-p35S:GFP), and delivered the loaded lysozyme-coated LDH30 nanoparticles to N. benthamiana roots according to the method described in Example 1.

### Results

Expression of the GFP protein was observed in both root tips and mature roots at 2 days post incubation with lysozyme-coated LDH30 nanoparticles for each of the treatment groups, with increased GFP intensity observed relative to untreated controls across the board (FIG. 40B). Interestingly, the p35S:GFP treatment group exhibited the highest intensity of GFP expression across the treatment groups in both root tip and mature root. Whilst only achieving statistical significance for root tips, the trend observed for both root tip and mature roots demonstrates that lysozyme-coated LDH delivery of a PCR-amplified transgene fragment induces higher expression of the transgene than the supercoiled or linearized plasmids encoding the same transgene.

### Conclusion

The inventors have shown that the Lysozyme-coated nanoparticle delivery system of the disclosure can be used to transfer transgene fragments to plant tissue, resulting in expression of the transgene in the plant tissue. Furthermore, lysozyme-coated LDH delivery of the transgene fragments induces higher expression of the transgene relative the expression of the transgene when delivered via a supercoiled or linearized plasmid.

### Example 5: NLS-Coated LDH30 Nanoparticles for Delivery and Expression of GFP Transgene In Arabidopsis Roots

### Materials and Methods

The materials and methods were the same as used in Example 1, with the exception of the following details.

A nuclear localisation signal (NLS) peptide derived from SV40 T-antigen having the amino acid sequence PKKKRKVEDPYC (SEQ ID NO: 5) was obtained from GeneScript. LDH30 nanoparticles were coated with the NLS peptide, by dropwise addition of LDH nanoparticle suspension (30 μg/mL) to a NLS peptide solution (1 mg/mL) under vigorous stirring conditions to achieve a final NLS peptide/LDH30 mass ratio of 5:1. The peptide coating/encapsulation was confirmed by FTIR consistent with previously Examples. In addition to the NLS-coated LDH30 nanoparticles (NLS@LDH), the inventors prepared LDH30 nanoparticles co-coated with the NLS peptide and Lysozyme (NLS&Lys@LDH), and LDH30 nanoparticles coated with Lysozyme (Lys@LDH). Coating of LDH nanoparticles with Lysozyme was performed in accordance with Example 1. Co-coating of LDH nanoparticles with NLS and Lysozyme was performed in accordance with Example 1, with the exception that the LDH nanoparticles were added to a solution comprising equal parts of Lysozyme and the NLS peptide. A linearized DNA plasmid comprising the GFP transgene (i.e., linearised pDNA-1 described in Example 1; 5 μg/mL) was then loaded onto the Lys@LDH30 nanoparticles, the NLS@LDH30 nanoparticles and the NLS&Lys@LDH30 nanoparticles (producing Lys@LDH30-pDNA, NLS@LDH30-pDNA and NLS&Lys@LDH30-pDNA, respectively).

Roots of Arabidopsis seedlings were incubated for 3 days in a suspension comprising one of the following LDH treatments: Lys@LDH30-pDNA, NLS@LDH30-pDNA, or NLS&Lys@LDH30-pDNA. Furthermore, roots from half of the Arabidopsis seedlings in each LDH treatment group were pretreated with 150 μg/ml Lysozyme (MES pH6.0) for 6 hours, and then washed, before being incubated for 3 days in the LDH suspensions. An untreated Control was also performed. The treatment groups were as follows:

### Untreated Control

Lys@LDH30-pDNA
NLS@LDH30-pDNA
NLS&Lys@LDH30-pDNA
Lys@LDH30-pDNA (Lys pre-treatment)
NLS@LDH30-pDNA (Lys pre-treatment)
NLS&Lys@LDH30-pDNA (Lys pre-treatment)

Confocal microscopy was then performed on whole seedlings, mature root (upper and lower) and root tips. Quantitative analyses of GFP fluorescence intensity was performed separately for mature root and root tips.

### Results

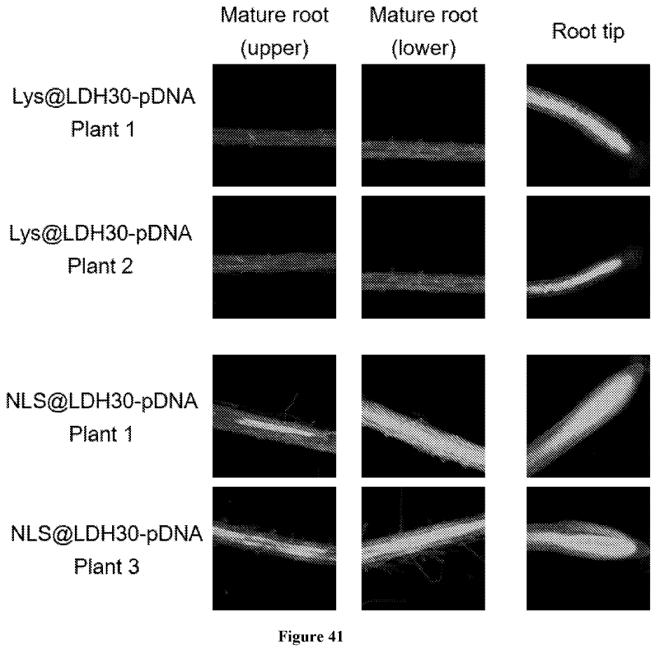

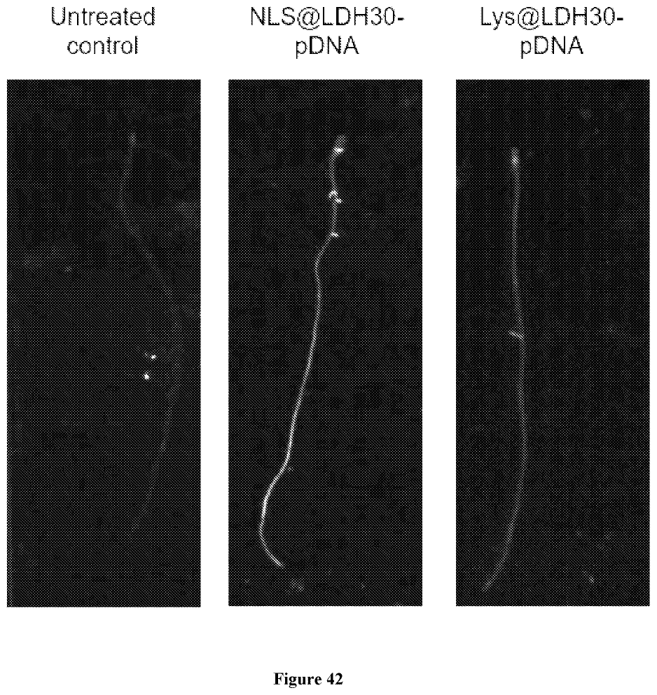

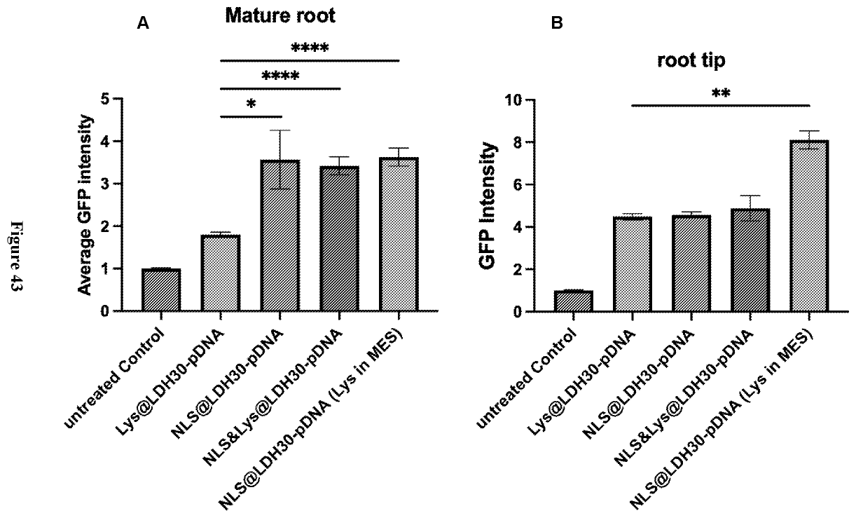

Confocal microscope images showing GFP expression in mature root (upper and lower) and root tips of Arabidopsis seedlings incubated with Lys@LDH30-pDNA or NLS@LDH30-pDNA is shown in FIG. 41. As is evident, coating of the LDH nanoparticles with either NLS peptide (NLS@LDH30-pDNA) or Lysozyme (Lys@LDH30-pDNA) resulted in uptake and internalisation of the LDH nanoparticles and expression of the GFP transgene in plant tissue. However, enhanced uptake and cellular internalisation of NLS@LDH30-pDNA relative to Lys@LDH30-pDNA was evident, particularly in the mature root tissue where GFP expression was more pronounced for the NLS@LDH30-pDNA treatment group (FIG. 41). This enhanced uptake and expression of GFP in the Arabidopsis roots treated with the NLS-coated LDH nanoparticles is also evident in FIG. 42.

Quantitative analysis of GFP fluorescence intensity for Arabidopsis roots treated with NLS@LDH30-pDNA or Lys@LDH30-pDNA told a similar story, with both treatment groups showing enhanced GFP fluorescence intensity relative to background florescence of the untreated Control in both mature root and root tips (FIG. 43), and the NLS@LDH30-pDNA treatment resulted in enhanced uptake and expression of GFP in mature root tissue relative to that of the Lys@LDH30-pDNA treatment group (FIG. 43A). Co-coating of the LDH30 nanoparticles with NLS and Lysozyme (NLS&Lys@LDH30-pDNA) did not result in any noticeable enhancement of GFP expression in root tissue relative to that observed when LDH30 nanoparticles were coated with NLS only (NLS@LDH30-pDNA). In this regard, the same trend towards enhanced uptake and expression of GFP in mature root tissue wase observed for NLS&Lys@LDH30-pDNA and NLS@LDH30-pDNA relative to the Lys@LDH30-pDNA treatment group (FIG. 43A), with comparable uptake and expression of GFP observed in root tips for all three of Lys@LDH30-pDNA, NLS@LDH30-pDNA, and NLS&Lys@LDH30-pDNA (FIG. 43B).

Interestingly, pre-treatment of the Arabidopsis roots with Lysozyme resulted in significantly enhanced uptake and expression of GFP in the root tips of plants treated with the NLS-coated LDH30 nanoparticles (NLS@LDH30-pDNA), but not in those plant treated with Lysozyme-coated LDH30 nanoparticles (Lys@LDH30-pDNA) or LDH30 nanoparticles co-coated in NLS peptide and Lysozyme (NLS&Lys@LDH30-pDNA) (FIG. 43B). This result suggests that there may be synergism at play between the pre-treatment of plant tissue with Lysozyme and subsequent treatment with NLS-coated LDH nanoparticles, which is independent of the effect of co-coating LDH nanoparticles with Lysozyme and NLS peptide. Without being bound by any one theory, the inventors believe that the enhanced uptake and internalisation observed for the NLS@LDH30-pDNA following pre-treatment with Lysozyme may due to the ability of the NLS-coated LDH nanoparticles to enter plant cells via endocytosis and localised within the cells, after which the LDH nanoparticles can access the membrane trafficking pathway of plants. In this regard, Lysozyme and NLS peptide, both of which are proteins having overall positive surface charges, are able to stimulate the membrane trafficking pathway and, in doing so, enhance uptake and internalisation of LDH nanoparticles on which the proteins are coated. This is supported by the inventors finding that both NLS- and Lysozyme-coated LDH nanoparticles can independently be taken up and internalised by plant tissues, and to a greater degree than their uncoated LDH nanoparticle counterpart. Additionally, and independent of the ability of the protein-coated LDH nanoparticles to access the membrane trafficking pathway, partial degradation of polysaccharides in the plant cell wall by Lysozyme pre-treatment allows LDH nanoparticles (coated or uncoated) to diffuse more freely through the apoplast and readily engage in endocytosis via the cell membrane. When a Lysozyme pre-treatment is combined with NLS-coating of the LDH nanoparticles, uptake and internalisation of the LDH nanoparticles is enhanced by the combined effect of engaging in endocytosis and stimulating the membrane trafficking pathway.

### Conclusion

This experiment demonstrated that the uptake and cellular internalisation of LDH nanoparticles by plant tissue is enhanced when the LDH nanoparticles are coated in NLS-peptide or Lysozyme. Furthermore, pre-treatment of roots with Lysozyme enhances the uptake and cellular internalisation of NLS-coated LDH nanoparticles but not Lys-coated LDH nanoparticles, suggesting that lysozyme pre-treatment may be useful for enhancing uptake and cellular internalisation of LDH nanoparticles coated with other proteins.

### Example 6: Lysozyme-Coated LDH Delivery of GFP mRNA Containing a Mobile tRNA Motif Enables Systemic Expression of the GFP Protein

In view of evidence in the literature that mobile transfer RNA (tRNA)-derived sequences are able to mediate transport of protein-coding mRNA via the phloem vasculature, including through graft junctions (e.g., Zhang et al (2016) Plant Cell 28:1237-1249), the inventors sought to determine whether their protein-coated LDH delivery system could be used to deliver mRNA encoding a protein of interest and containing a mobile tRNA motif to facilitate systemic expression of the protein.

### Materials and Methods

The materials and methods were the same as used in Examples 1-5, with the exception of the following details.

The pDNA-1 described in Example 1 was modified to include an eGFP: tRNA fusion construct capable of expressing GFP;tRNA fusion product. Briefly, an eGFP: IRNA fusion construct comprising the eGFP cassette of pDNA-1 and harbouring a tRNA variant in the 3′ UTR was created by PCR according to the methodology described in Zhang et al (2016) Plant Cell 28:1237-1249 and cloned into the pUQC10196 vector between the p35S promoter and ocs element to produce the modified pDNA-1 (hereinafter ‘pDNA-Imod’).

The pDNA-1mod or pDNA-1 was then loaded onto Lys@LDH30 nanoparticles using the method described in Example 1 to produce Lys@LDH30-pDNA-1mod and Lys@LDH30-pDNA-1, respectively.

Confocal microscopy was then performed on whole seedlings, mature root (upper and lower) and root tips. Quantitative analyses of GFP fluorescence intensity was performed separately for root tips, mature root, stem, and leaf.

### Results

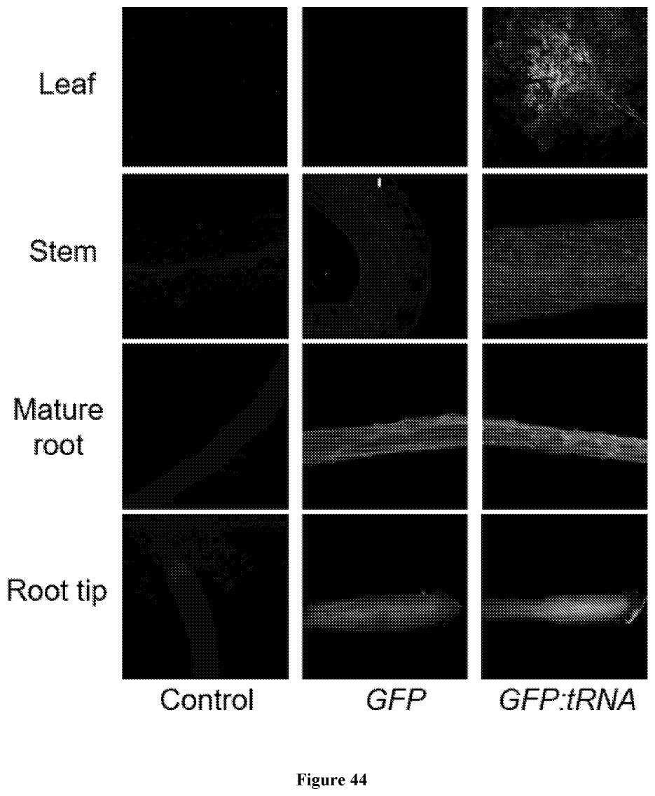

Confocal microscope images showing GFP expression in root tips, mature root, stem, and leaf of Control Arabidopsis seedlings and Arabidopsis seedlings incubated with Lys@LDH30-pDNA-1 or Lys@LDH30-pDNA-1mod is shown in FIG. 44. As is evident, the Lysozyme-coated LDH nanoparticle delivery system resulted in effective uptake and internalisation of plasmid DNA loaded onto the LDH30 nanoparticles consistent with the inventors findings in Examples 1-5. Uptake and internalisation of Lys@LDH30-pDNA-1 by the roots of Arabidopsis seedlings resulted in strong expression of GFP in root tips and mature root, with minor expression of GFP in the stem and no visible expression in the leaf (FIG. 44), consistent with the results of earlier experiments.

### Conclusion

This experiment demonstrates that the inventors' protein-coated LDH delivery system can be used to effectively deliver plasmid DNA encoding a functional mRNA transcript for a protein of interest containing a mobile tRNA motif, and that systemic expression of the protein can be achieved.

1. A composition comprising one or more layered double hydroxide (LDH) nanoparticles onto which one or more nucleic acid molecules are adsorbed and coated in a protein having an overall positive surface charge.

2. A method for preparing the composition of claim 1, comprising adsorbing one or more nucleic acids molecules onto a LDH nanoparticle and then coating the LDH nanoparticle with a protein that has an overall positive surface charge.

3. The method of claim 2, wherein the protein is coated onto the one or more LDH nanoparticle through electrostatic interactions.

4. A method of introducing one or more nucleic acid molecules to a plant tissue, comprising:
(i) contacting the plant tissue with a protein having an overall positive surface charge; and
(ii) contacting the plant tissue with a layered double hydroxide (LDH) nanoparticle onto which the nucleic acid molecules are adsorbed.

5. The method of claim 4, wherein the protein and LDH nanoparticles are contacted with the plant tissue separately.

6. The method of claim 5, wherein the protein and LDH nanoparticles are contacted with the plant tissue simultaneously.

7. The method of claim 5, wherein the protein and LDH nanoparticles are contacted with the plant consecutively, optionally wherein the plant tissue is contacted with the protein prior to being contacted with the one or more LDH nanoparticles.

8. The composition of claim 1, wherein the one or more nucleic acid molecules are single-stranded or double-stranded, optionally wherein the one or more nucleic acid molecules are selected from the group consisting of a messenger RNA (mRNA), a double stranded RNA (dsRNA), a short interfering RNA (siRNA), single stranded RNA (ssRNA), short hairpin RNA (shRNA), short hairpin microRNA (shmiR), transfer RNA (tRNA), microRNA (miRNA), ribosomal RNA (rRNA), small guide RNA (sgRNA), a plasmid DNA (pDNA) and any combination thereof.

9. The method of claim 2, wherein the one or more nucleic acid molecules are single-stranded or double-stranded, optionally wherein the one or more nucleic acid molecules are selected from the group consisting of a messenger RNA (mRNA), a double stranded RNA (dsRNA), a short interfering RNA (siRNA), single stranded RNA (ssRNA), short hairpin RNA (shRNA), short hairpin microRNA (shmiR), transfer RNA (tRNA), microRNA (miRNA), ribosomal RNA (rRNA), small guide RNA (sgRNA), a plasmid DNA (pDNA) and any combination thereof.

10. The composition of claim 1, wherein (i) the protein is capable of degrading polysaccharides in a plant cell wall or (ii) the protein is lysozyme or Nuclear Localization Signal (NLS) peptide.

11. The method of claim 2, wherein (i) the protein is capable of degrading polysaccharides in a plant cell wall or (ii) the protein is lysozyme or Nuclear Localization Signal (NLS) peptide.

12. The composition of claim 1, wherein;
(i) the one or more LDH nanoparticles have an average diameter from about 30 nm to about 50 nm; and/or
(ii) the one or more LDH nanoparticles are coated with the protein at a mass ratio of about 1:5.

13. The method of claim 2, wherein:
(i) the one or more LDH nanoparticles have an average diameter from about 30 nm to about 50 nm; and/or
(ii) the one or more LDH nanoparticles are coated with the protein at a mass ratio of about 1:5.

14. The method of claim 4, wherein the protein and one or more LDH nanoparticles are contacted with the plant in the form of the composition of claim 1.

15. The method of claim 4, comprising immersing the plant tissue in an incubation medium comprising the LDH nanoparticles in the presence of the protein.

16. The method of claim 4, wherein:
(i) the plant is a monocot or a dicot; and/or
(ii) the plant is selected from the group consisting of a fruiting plant, a leguminous plant, an oil plant, a vegetable plant, a cereal plant, a fibre plant, an ornamental plant, a forestry plant, an aquatic plant, a medicinal plant and a noxious plant or weed.

17. (canceled)

18. The method of claim 4, wherein the plant tissue is root tissue or pollen.

19. The method of claim 4, wherein the LDH nanoparticles are internalized into the plant tissue and the nucleic acids adsorbed to the LDH nanoparticles are delivered to the plant tissue, optionally wherein the nucleic acid molecules are taken up via the membrane trafficking pathway of the plant.

20. The method of claim 4, wherein:
(i) internalization of the nucleic acid molecules by the plant tissue following performance of the method is increased relative to a method in which the plant tissue is contacted with the LDH nanoparticles in the absence of the protein having an overall positive surface charge; and/or
(ii) aggregation of the LDH nanoparticles is decreased relative to a method in which the plant tissue is contacted with the LDH nanoparticles in the absence of the protein.

21. The method of claim 4, wherein:
(i) the nucleic acid molecules silence a gene in the plant, or edit the plant's genome or increase expression of a gene in the plant; and/or
(ii) introduction of the nucleic acid molecule to the plant tissue increases yield, quality and/or harvest value of the plant and/or protects the plant from a plant pest and/or improves resistance of the plant to an abiotic stress and/or a biotic stress, optionally wherein the plant pest is an insect pest, a viral pathogen, a fungal pathogen, a nematode, a bacterial pathogen or a parasite.

22-23. (canceled)
